(19) United States
(12) Patent Application Publication
(10) Pub. No.: US 2012/0225079 A1
(43) Pub. Date: Sep. 6, 2012

(54) IMMUNIZING COMPOSITION FOR REDUCING STREPTOCOCCAL INFECTIONS

(75) Inventors: Bengt Guss, Uppsala (SE); Lars Frykberg, Storvreta (SE); Jan-Ingmar Flock, Bromma (SE); Margareta Flock, Bromma (SE)

(73) Assignee: INTERVACC AB, Hagersten (SE)

(21) Appl. No.: 13/509,576

(22) PCT Filed: Nov. 11, 2010
(86) PCT No.: PCT/SE2010/051238

§ 371 Date: May 11, 2012

Related U.S. Application Data
(60) Provisional application No. 61/261,026, filed on Nov. 13, 2009.

(51) Int. Cl. A61K 39/395; A61P 37/04; C12P 21/02; C07K 14/315; A61K 39/09

(52) U.S. Cl. 424/139.1; 530/350; 424/190.1; 424/244.1; 435/69.3

(57) ABSTRACT

An antigenic composition comprises at least one antigen, wherein said at least one antigen comprises at least part of a protein selected from EndoSe of Streptococcus equi subsp. Equi, EndoSz of Streptococcus equi subsp. zooepidemicus, and Endo S of Streptococcus pyogenes, and wherein said at least part of said protein comprises at least one antigenic epitope. A vaccine composition comprising the antigenic composition as immunizing component, methods for producing the antigeninc composition and the vaccine composition, use of the vaccine composition for prophylactic and therapeutic treatment, and an antibody preparation comprising monoclonal or polyclonal antibodies specific for an antigen or antigens of the antigenic composition are also disclosed.

### FIELD OF THE INVENTION

This invention relates to subunit immunogenic or vaccine compositions and use thereof for immunization of mammals susceptible to streptococcal infections. The invention also relates to methods for preparing, formulating and administrating such compositions.

### BACKGROUND OF THE INVENTION

Streptococcal infections in horses are mainly caused by the species Streptococcus equi, which is classified as a Lancefield Group C Streptococcus and comprises three subspecies designated equi, zooepidemicus, and ruminatorium respectively (Refs.15, 24, 40).

Streptococcus equi subsp. equi, which is virtually confined to horses, is the causative agent of strangles, a world-wide distributed and highly contagious serious disease of the upper respiratory tract of the Equidae. Strangles is one of the most frequently reported equine diseases world-wide and is characterized by fever, nasal discharge, and abscess formation in the retropharyngeal and mandibular lymph nodes. In some cases the disease shows a metastatic course in the body, so called “bastard strangles”. The disease has a world-wide distribution and causes great economic losses. Moreover, since strangles is a highly contagious disease, not only infected animals but also all other members of e.g. an afflicted stud must be isolated for as long as up to three months (Ref. 39).

S. equi subsp. zooepidemicus is considered as an opportunistic commensal often occurring in the upper respiratory tract of healthy horses. However, after stress or virus infection, it can cause a secondary infection, which results in strangles-like symptoms. Moreover, subsp. zooepidemicus infects not only horses but also a wide range of other animals, like pigs, dogs, goats, cats, and cows. Even human cases of infection due to subsp. zooepidemicus have been reported (Ref. 5). This subspecies has been implicated as the primary pathogen in conditions such as endometritis, cervicitis, abortion, mastitis, pneumonia, abscesses and joint infections.

The third subspecies ruminatorium has been isolated from milk of sheep and goats with mastitis (Ref. 10).

When used generally herein, the expression “S. equi” refers to one or both of subsp. equi and subsp. zooepidemicus.

Streptococcus pyogenes is an important human pathogen which causes a variety of diseases e.g. impetigo, pharyngitis, necrotizing fasciitis and toxic shock syndrome.

Although it is possible to treat and cure these streptococcal infections with antibiotics, such as penicillin, tetracycline or gentamicin, an effective prophylactic agent that could prevent outbursts of such infections and obviate or reduce the risk for development of resistant strains associated with antibiotic treatment would be appreciated.

### DESCRIPTION OF THE RELATED ART

However, although many attempts have been made to develop prophylactic agents such as vaccines against S. equi, at the present time no efficient and safe vaccines are available on the market, neither for the subsp. equi nor for the subsp. zooepidemicus, subsp. ruminatorium or S. pyogenes.

Existing vaccines against strangles are based on inactivated, e.g. heat-killed, or attenuated strains of S. equi subsp. equi or acid extracts/mutanolysin enriched in M-protein(s), i.e. immunogenic protein(s) produced by S. equi. A vaccine against S. equi subsp. zooepidemicus based on an M-like protein is disclosed in U.S. Pat. No. 5,583,014. In WO 87/00436, Ref. 17 and WO 2009/7093014 A2 attenuated strains of S. equi are disclosed for use as a vaccine against infections caused by S. equi.

Recently, a commercial vaccine against strangles, Equilis StrepE from Intervet, UK, has been released in Great Britain (November 2004), which vaccine also has been used throughout Europe and in South Africa and South America. However, the safety and efficacy of this vaccine, which is based on an attenuated (living, deletion mutated) strain of S. equi subsp. equi, can be questioned (Refs. 23, 35).

Since the previously developed vaccines or immunizing preparations based on living or inactivated bacteria are hampered by side-effects and may provide insufficient protection there is a need for efficient and safe prophylactic agents, such as vaccines, that protect against S. equi infections and/or prevent spread thereof without giving rise to undesirable side-effects.

For years, streptococcal surface proteins that interact with and/or bind to different components of the Extracellular Matrix (ECM) or plasma proteins of the host cell have been identified and characterized. Examples of extracellular surface proteins of S. equi that have been characterized are FNZ (Ref. 29), EAG (Ref. 27), ScIC (Ref. 21), CNE (also called Sec) (Ref. 25), ZAG (Ref. 18 and WO 95/07296). Furthermore, examples of S. equi extracellular proteins that are supposed to be released into the surrounding medium are SFS (Ref. 28), IdeE and IdeZ (Ref. 26), IdeE2 and IdeZ2 (Ref. 16). These types of proteins are potential candidates for use as active component(s) for immunizing purposes

The uses of this type of proteins as components in a potential vaccine for protection of horses against strangles are disclosed in WO 2004/032957 A1, WO 00/37496, WO 2007/115059 A2 and WO 98/01561.

In Flock, M., et al (2004) (Ref. 11), it is reported that in a mouse model of equine strangles, parts of the proteins designated FNZ, SFS and EAG, respectively, were used to immunize mice. FNZ and EAG were considered as promising candidates for development of a safe and efficacious vaccine against strangles.

In Timoney et al (2007) (Ref. 42) it is reported that recombinant DNA produced extracellular proteins of subsp. equi are useless as vaccine components. It was speculated therein that earlier reported results for some S. equi proteins produced by recombinant DNA technology, showing protection in mice experiments, are not applicable to horses. Thus, it is not obvious that recombinant forms of extracellularly localized S. equi proteins necessarily are likely candidates as vaccine components.

In Ref. 45, vaccination of horses against strangles using the recombinant antigens EAG, CNE and ScIC from S. equi subsp. equi is reported. In this study, vaccinated horses showed, after challenge with S. equi subspecies equi, significantly reduced recovery of bacteria and significantly lower levels of nasal discharge.

Although many efforts have been made to develop efficient vaccines and some of the immunizing components presented in Refs. 14 and 15, WO 2004/032957 A1 and WO 2009/075646 A1 are promising candidates for use in a vaccine that protects against S. equi infection, development of safe vaccines having a high degree of immunogenicity and exhibiting limited or no side effects is still desirable.

The human pathogen Streptococcus pyogenes also expresses a great number of extracellular proteins interacting with ECM and/or blood components of the host (Refs. 6, 7, 9, 33). Among these are an endoglycosidase, called EndoS that has the ability to hydrolyse the chitobiose core of the asparagine-linked glycan on human immunoglobulin G (IgG) (Ref. 8). EndoS has been further characterized in a series of articles, describing e.g. enzymatic properties, specificity etc (Refs. 1, 2, 3, 4, 34). The use of EndoS in treating or preventing diseases mediated by IgG antibodies such as autoimmune diseases is disclosed in WO 2008/071418 A2 and the in vitro use of EndoS to isolate and analyse IgG is disclosed in WO 2009/033670 A2. However, the use of EndoS, or EndoS-like proteins or fragments thereof, as a component in a vaccine against bacterial infections is not described. Nor is the use of EndoS or EndoS-like proteins or fragments thereof to elicit an immunogenic response or a protective immune response disclosed in WO 2008/071418 A2 or WO 2009/033670 A2.

### BRIEF SUMMARY OF THE INVENTION

The present invention is based on an antigenic, suitably an immunogenic, composition comprising at least one antigen, suitably an immunogen, that comprises at least one antigenic epitope or antigenic determinant derived from the EndoSe protein present in S. equi subsp. equi and/or the EndoSz protein present in S. equi subsp. zooepidemicus and use thereof for immunization of mammals (including humans) against S. equi subsp. equi and/or subsp. zooepidemicus.

The present invention is also directed to a subunit immunogen or vaccine composition comprising at least one antigen, suitably an immunogen, that comprises at least one antigenic epitope or antigenic determinant derived from the EndoSe protein present in S. equi subsp. equi and/or the EndoSz protein present in S. equi subsp. zooepidemicus and/or the EndoS protein present in S. pyogenes.

The present invention is further directed to such antigenic compositions as immunizing components; to methods to prepare said antigenic, suitably immunogenic, compositions or vaccine compositions; to methods to induce an immune response against S. equi and/or S. pyogenes in non-human mammals and optionally also in humans; and to methods for prophylactic or therapeutic treatment of S. equi and/or S. pyogenes infection in non-human mammals and optionally also in humans.

As mentioned above, when used generally herein, the expression “S. equi” refers to one or both of subsp. equi and subsp. zooepidemicus.

According to a suitable embodiment, the present invention is directed to a vaccine that protects equines, such as horses, against diseases caused by S. equi, e.g. strangles, upper respiratory tract infections, wound infections and endometritis. The word “protects” is a general term including anything between full protection and reduction of the severity of infection. The degree of protection can be measured in various ways. Concerning e.g. S. equi subsp. equi infections in horses the effect of the vaccine can be reduced clinical symptoms and reduced clinical disease, where reduced increase in temperature, reduced swelling of lympnodes and reduced dissemination of bacteria from infected animals etc can be observed. Methods and procedures how to measure the efficacy of an immunizing composition after challenge can be obtained from e.g. Ref.14, and WO 2009/075646 A1.

For various reasons, before performing vaccination and challenge experiments in horses, the evaluation of novel antigens to be used in a vaccine are studied in a small animal model. Concerning upper respiratory tract infections caused by subsp. equi a suitable and well established vaccination and experimental infection model has been described (Refs. 11, 12, 13, 14, 16, 43, WO 2004/032957 A1, WO 2009/075646 A1). This model has been used with a high degree of reliability to screen and evaluate S. equi antigens with a potential to provoke a protective immunogenic response in horses (Refs. 13, 14).

In the context of infections caused by S. equi subsp. equi, the expression “non-human mammals” primarily refers to animals belonging to the family Equidae that consists of horses, donkeys and zebras and to hybrids thereof, such as mules and hinnies. Camels and dromedaries are also encompassed therein.

In connection with infections caused by S. equi subsp. zooepidemicus, the expression “non-human mammals” in addition refers also to other mammals such as cows, pigs, sheep, goats, dogs and cats.

In particular embodiments, the present invention makes use of one or more polypeptides selected from the amino acid sequences of SEQ ID NOS: 2, 4, 6, 8, 9, 11, 13, 15, 17 and one or more nucleotide sequences selected from the nucleotide sequences of SEQ ID NOS: 1, 3, 5, 7, 10, 12, 14, 16.

The practice of the present invention will employ, unless otherwise indicated, conventional techniques of microbiology, recombinant DNA technology and molecular biology and immunology, which are within the skills of the art. Such techniques are explained in literature, e.g. Sambrook et al (2001) Molecular Cloning: A laboratory manual, 3^{rd} ed. Cold Spring Harbour Press. Unless defined otherwise, all scientific and technical terms used herein have the same meaning as commonly understood by a person with ordinary skill in the art to which the invention pertains.

A “fragment” of a molecule such as a protein or nucleic acid is meant to refer to any portion of the amino acid or nucleotide sequence.

The term “analog” refers to a nucleic acid or amino acid sequence variant having a sequence homology (“identity”) of 80% or more, especially 90% or more, with the reference sequence. In general, “identity” refers to an exact nucleotide to nucleotide or amino acid to amino acid correspondence of two polynucleotides or polypeptide sequences, respectively. Techniques for determining nucleic acid and amino acid sequence identity are well known in the art, and software programs for calculating identity between sequences are available. Analogs to the EndoSe and EndoSz proteins, for example, will include any corresponding “Endo” protein of the S. equi subspecies ruminatorium.

### BRIEF DESCRIPTION OF THE DRAWINGS

In the following, the present invention is described in closer detail with reference to the drawings, where:

FIG. 1 shows the average weight loss of mice infected with S. equi subsp. equi. The mice (n=10) had previously been vaccinated with antigens as indicated. Mean values and standard errors are shown.

FIG. 2 shows the nasal growth of S. equi subsp. equi of mice infected S. equi subsp. equi. The mice (n=10) had previously been vaccinated with antigens as indicated. Mean values and standard errors are shown.

FIG. 3 shows antibody titer against CNE in mice (n=10) immunized with CNE or CNE+EndoSe as indicated. Mean values and standard errors of log values of dilutions required to get an absorbance of 1.5 in ELISA are shown. Values from non-vaccinated mice are included.

FIG. 4 shows antibody titer against EndoSe in mice (n=10) immunized with CNE+EndoSe. Mean values and standard errors of log values of dilutions required to get an absorbance of 1.5 in ELISA are shown. Values from non-vaccinated mice are included.

FIG. 5 shows the average weight loss of mice infected with S. equi subsp. equi. The mice (n=10) had previously been vaccinated with antigens as indicated. Mean values and standard errors are shown.

FIG. 6 shows the nasal growth of S. equi subsp. equi of mice infected with S. equi subsp. equi. The mice (n=10) had previously been vaccinated with antigens as indicated. Mean values and standard errors are shown.

FIG. 7 shows the average weight loss of mice infected with S. equi subsp. equi. The mice (n=8) had previously been vaccinated with antigens as indicated. Mean values and standard errors are shown.

FIG. 8 shows the nasal growth of S. equi subsp. equi of mice infected with S. equi subsp. equi. The mice (n=8) had previously been vaccinated with antigens as indicated. Mean values and standard errors are shown.

FIG. 9 shows ClustalW2 alignment of Endo-proteins. MGCS10565 is the endo-beta-N-acetylglucosaminidase F2 precursor of S. equi subsp. zooepidemicus (NCBI Reference Sequence: YP_{—}002122753.1); NO11 is SEQ ID NO:11; NO2 is SEQ ID NO: 2; NO15 is SEQ ID NO: 15. Below the alignment a consensus line is also displayed. The following symbols denote the degree of conservation observed in each column: ‘*’, identical residues in all sequences; ‘:’, highly conserved column; ‘.’, weakly conserved column.

FIG. 10 shows the average weight loss of mice infected with S. zooepidemicus. The mice (n=15) had previously been vaccinated with EndoSe. Mean values and standard errors are shown.

FIG. 11 shows the nasal growth of S. equi subsp. zooepidemicus of mice infected with S. equi subsp. zooepidemicus. The mice (n=15) had previously been vaccinated with EndoSe. Mean values and standard errors are shown.

FIG. 12 shows the average weight loss of mice infected with S. pyogenes. The mice (n=15) had previously been vaccinated with EndoSe. Mean values and standard errors are shown.

FIG. 13 shows the nasal growth of S. pyogenes of mice infected with S. pyogenes. The mice (n=15) had previously been vaccinated with EndoSe. Mean values and standard errors are shown.

FIG. 14 shows the ability of antiserum against EndoSe, at indicated concentrations, to inhibit the function of EndoSe to prevent IgG from binding to immobilized EndoSe. With higher concentrations of anti serum against EndoSe (squares) the binding of IgG is restored. Negative serum (circles) has no such effect. Mean values and standard errors are shown from sera from six mice. Y-axis shows binding of IgG determined as absorbance (492 nm).

### BRIEF DESCRIPTION OF THE SEQUENCE LISTING

SEQ ID NO: 1 shows the nucleotide sequence of the gene endoSe.

SEQ ID NO: 2 shows the amino acid sequence of the protein EndoSe.

SEQ ID NO: 3 shows the nucleotide sequence of the gene endoSe encoding recombinant EndoSe (lacking the nucleotide sequence encoding the signal sequence).

SEQ ID NO: 4 shows the amino acid sequence of the recombinant protein EndoSe (encoded by SEQ ID NO: 3).

SEQ ID NO: 5 shows the nucleotide sequence coding for fragment A of endoSe.

SEQ ID NO: 6 shows the amino acid sequence of the recombinant protein fragment A of EndoSe.

SEQ ID NO: 7 shows the nucleotide sequence coding for fragment C of endoSe.

SEQ ID NO: 8 shows the amino acid sequence of the recombinant protein fragment C of EndoSe.

SEQ ID NO: 9 shows the amino acid sequence of recombinant fragment SEC 2.16 of CNE.

SEQ ID NO: 10 shows the nucleotide sequence of the gene endoSz from subsp. zooepidemicus.

SEQ ID NO: 11 shows the amino acid sequence of the protein EndoSz.

SEQ ID NO: 12 shows the nucleotide sequence of the gene endoSz from subsp. zooepidemicus encoding recombinant EndoSz lacking the signal sequence.

SEQ ID NO: 13 shows the the amino acid sequence of recombinant EndoSz encoded by SEQ ID NO: 12.

SEQ ID NO: 14 shows the nucleotide sequence of the ndos gene endoding EndoS (truncated sequence of GenBank: AF296340.1).

SEQ ID NO: 15 shows the amino acid sequence of EndoS from S. pyogenes (GeneBank: AAK00850.1).

SEQ ID NO: 16 shows the nucleotide sequence of the endoS gene (SEQ ID NO: 14) lacking the sequence encoding the signal sequence.

SEQ ID NO: 17 shows the amino acid sequence of EndoS from S. pyogenes endoded by SEQ ID NO: 16.

SEQ ID NOS: 18-25 in Table 1 show nucleotide sequences of oligonucleotide primers.

### DETAILED DESCRIPTION OF THE INVENTION

As mentioned above, the present invention is concerned with identification of polypeptides or proteins of S. equi or S. pyogenes that are able to elicit an antigenic, suitably an immunogenic, response, when administered to a mammal, and to the identification of polynucleotides or genes encoding these polypeptides or proteins.

The present invention is also concerned with fragments or analogs of said polypeptides or proteins or of said polynucleotides or genes.

More specifically, the genes of S. equi encoding EndoSe and fragments thereof were identified and, subsequently, the corresponding products were expressed and evaluated in vaccine studies. The present invention is based on such studies.

Accordingly, the present invention relates to an antigenic composition comprising at least one antigen, wherein said at least one antigen comprises at least part of the EndoSe protein of S. equi subsp. equi or EndoSz of subsp. zooepidemicus, and said at least part of said protein comprises at least one antigenic epitope or antigenic determinant of S. equi.

According to one embodiment, the present invention is directed to an antigenic composition comprising at least one antigen (EndoSe alt. EndoSz), wherein said at least one antigen comprises at least part of a protein or polypeptide of S. equi subsp. equi or subsp. zooepidemicus and said at least part of said protein or polypeptide comprises at least one antigenic epitope or antigenic determinant of S. equi, and wherein said at least part of a protein or polypeptide is selected from the group comprising:

a protein or polypeptide which is designated EndoSe and has an amino acid sequence as shown in SEQ ID NO: 4;

a protein or polypeptide which is designated fragment A of EndoSe and has an amino acid sequence as shown in SEQ ID NO: 6;

a protein or polypeptide which is designated fragment C of EndoSe and has an amino acid sequence as shown in SEQ ID NO: 8;

a protein or polypeptide which is designated EndoSz and has an amino acid sequence as shown in SEQ ID NO:13.

The above-mentioned antigen or antigens may further be combined with a protein or polypeptide selected from the group comprising:

a protein or polypeptide which is designated CNE and has an amino acid sequence as shown in WO 2004/032957 A1;

a protein or polypeptide which is designated FNZ and has an amino acid sequence as shown in WO 2004/032957 A1;

a protein or polypeptide which is designated SFS and has an amino acid sequence as shown in WO 2004/032957 A1;

a protein or polypeptide which is designated ScIC and has an amino acid sequence as shown in WO 2004/032957 A1;

a protein or polypeptide which is designated EAG and has an amino acid sequence as shown in WO 2009/075646 A1, SEQ ID NO: 13;

a protein or polypeptide which is designated IdeE and has an amino acid sequence as shown in WO 2009/075646 A1, SEQ ID NO: 10;

a protein or polypeptide which is designated IdeE2 and has an amino acid sequence as shown in WO 2009/075646 A1, SEQ ID NO: 1;

a protein or polypeptide which is designated Eq5 and has an amino acid sequence as shown in WO 2009/075646 A1, SEQ ID NO: 3;

a protein or polypeptide which is designated Eq8 and has an amino acid sequence as shown in WO 2009/075646 A1, SEQ ID NO: 5;

a protein or polypeptide which is designated IdeZ2 and has an amino acid sequence as shown in WO 2009/075646 A1, SEQ ID NO: 7;

a protein or polypeptide which is designated Eqz5 and has an amino acid sequence as shown in WO 2009/075646 A1, SEQ ID NO: 8; and

a protein or polypeptide which is designated Eqz8 and has an amino acid sequence as shown in WO 2009/075646 A1, SEQ ID NO: 9; or an analog or a fragment thereof.

For convenience, the polypeptides having amino acid sequences as shown in the sequence listing of WO 2009/075646 A1 and WO 2004/032957 A1 are frequently only designated CNE, FNZ, ScIC, SFS, EAG, IdeE, IdeE2, Eq5, Eq8, IdeZ2, Eqz5, and Eqz8, respectively. EAG, IdeE, IdeE2, Eq5, and Eq8 designate proteins that can be found in S. equi subsp. equi, and IdeZ2, Eqz5 and Eqz8 designate proteins that can be found in S. equi subsp. zooepidemicus. Other examples are the M or M-like proteins, e.g. SeM described in Ref. 42.

However, the proteins or polypeptide fragments that may be included in the antigenic compositions of the invention are not restricted to those listed above. In general, the invention can be used in principle with any extracellular protein or fragments thereof expressed on the surface of pathogenic streptococci, e.g. different subsp. of S. equi or S. pyogenes, or proteins transported into the environment. By DNA sequence analysis of the genome of these bacteria, e.g. http://www.sanger.ac.uk/Projects/S equi/; http://www.sanger.ac.uk/Projects/S zooepidemicus/; http://www.sanger.ac.uk/Projects/S pyogenes/, open reading frames can be identified coding for extracellular proteins. These proteins are usually characterized by harboring an N-terminal signal sequence responsible for the transport across the membrane after translation. A particular interesting group of protein for vaccine development are proteins which in addition to harboring the signal sequence also display an easily recognized C-terminal domain including an amino acid motif generally defined as e.g. LPXTG, important for anchoring an extracellular protein to the peptidoglycan structure of the bacterial cell wall (Ref. 37). How to identify such proteins by bioinformatics methods, e.g. computer program SignalP (http://www.cbs.dtu.dk/services/SignalP/), (Refs.19, 38) is well known to people skilled in the art.

The antigens or immunogens of the present antigenic or immunogenic compositions may comprise the entire amino acid sequence of said protein or polypeptide or may comprise a fragment, e.g. a C-terminal or N-terminal fragment thereof, or an analog thereof. For instance, an N-terminal fragment and a C-terminal fragment of EndoSe (or EndoSz or EndoS) are used according to various embodiments of the present invention.

The present invention is also related to an antigenic composition comprising at least one antigen, wherein said at least one protein or polypeptide is selected from the group consisting of EndoSe, EndoSz and EndoS, and which composition further comprises at least one antigen, which is selected from the group comprising a protein or a polypeptide of extracellular proteins, e.g. CNE, ScIC, SFS, FNZ, EAG, Eq5, Eq8, IdeE, IdeE2, IdeZ2, Eqz5, Eqz8, the ScIC proteins ScID-Scll (genbank acc. nos. DQ158080, DQ158081, DQ158082, DQ158083, DQ158084, DQ158085), FNE (acc. no. AF360373), FNEB (acc. no AY898649) FNEC-FNEF (Ref. 24), SeM (acc. no.U73162, also called FBP acc.no. YP002747233), SzPSe (acc. no. U73162), seeH (acc. no. AF186180), seeM (acc. no. AJ583528), seel (GenBank. Gene ID7697191, SEQ2037, Ref. 15), seeL (acc. no. AJ583527), Se51.9 (acc. no. AF521601), Se46.8 (acc. no. AF521600), Se24.3 (acc. no. AY137521), Se75.3 (acc. no. AY137528), Sell0,0 (acc. no. AY137519), Se24.3 (AY137521), Se42.0 (acc. no AY137521), Se117.0 (acc.no. AY137523), Se18.9 (acc. no. DQ068464), ZAG (acc. no. U25852), slaA (acc. no. CAW93317), slaB (acc. no. CAW95519), sagA (acc. no. ACG61862), streptolysin S biosynthesis proteins (CW92800, CW92802, CW92798), streptolysin S precursor (CW92796), SpyCEP (acc. no. DQ413032), the SpyCEP similar proteins SeCEP and SzoCEP (Ref. 43).

In this antigenic composition, said at least one protein or polypeptide may advantageously be selected from the group consisting of:

a protein or polypeptide which is designated EndoSe and has an amino acid sequence as shown in SEQ ID NO 4;

a protein or polypeptide which is designated fragment A of EndoSe and has an amino acid sequence as shown in SEQ ID NO 6;

a protein or polypeptide which is designated fragment C of EndoSe and has an amino acid sequence as shown in SEQ ID NO 8;

a protein or polypeptide which is designated EndoSz and has an amino acid sequence as shown in SEQ ID NO13.

a protein or polypeptide which is designated EndoS and has an amino acid as shown in SEQ ID NO 17.

According to the present invention, the antigenic compositions suitably comprise at least one antigen which is produced by recombinant technology and/or at least one antigen which is an isolated or purified antigen. However, the present invention is not restricted to recombinant forms of antigens, e.g. EndoSe, EndoSz or EndoS proteins or fragments thereof. Alternative sources of these proteins (or fragments thereof) are the native forms produced by the streptococcal bacteria (or overproducing mutants). The native forms may be isolated from cells or growth media from bacteria grown in suitable media resulting in high production of the respective protein. In addition, after finding the optimal growth conditions (including physiological conditions) to obtain the native proteins it is also possible to construct overproducing streptococcal strains. Using methods well known to people skilled in the art there are several ways to generate and isolate overproducing strains, e.g. by site directed mutagenesis, chemical mutagenesis, ultraviolet light etc. The procedure of purifying and isolating an extracellullar protein from growth media is well known to people skilled in the art.

From the above, it is evident that the present antigens or immunogens that are derived from proteins of S. equi or S. pyogenes may comprise the entire protein, a fragment of said protein or an analog of said protein (like for instance synthetic peptides) which is antigenic or immunogenic. Thus, the present invention is not limited to the fragments of proteins that are specifically disclosed herein.

The antigenic composition of the present invention may comprise at least one recombinant vector and at least one polynucleotide inserted therein that encodes said at least one protein or polypeptide, and which vector is able to express said polypeptide in vivo in a non-human mammal susceptible to infection with S. equi.

According to one embodiment of the present invention, the vector is an expression vector which is a plasmid or a viral vector and wherein said polynucleotide has a nucleotide sequence that encodes an antigen of the present invention.

The application of the present invention is not restricted to the usage of E. coli and vectors suitable for this bacterium as vehicles and tools to express recombinant polypeptides. Other hosts and vectors are well known in the art and can be found in literature and in literature cited in WO 2007/115059 A2.

A further embodiment of the present invention is concerned with a vaccine composition for protecting non-human mammals against infection of S. equi, which comprises an antigenic composition as disclosed above as immunizing component, and a pharmaceutically acceptable carrier.

Suitably, the present vaccine composition comprises an antigenic or immunogenic composition that contains one or more of the present antigens or immunogens as immunizing component(s). Optionally, one or more of these antigens or immunogens are comprised of analogs of said proteins or fragments thereof, e.g. N-terminal or C-terminal fragments.

The vaccine composition may comprise further components, such as an adjuvant. Suitably, the adjuvant stimulates systemic or mucosal immunity. Such adjuvants are well known in the art.

Suitable adjuvants for use according to the present invention comprise (1) polymers of acrylic or methacrylic acid, maleic anhydride and alkenyl derivative polymers, (2) immunostimulating sequences (ISS), (3) an oil in water emulsion, (4) cation lipids containing a quaternary ammonium salt, (5) cytokines, (6) aluminum hydroxide or aluminum phosphate, (7) saponin or (8) nanoparticles or (9) any combinations or mixtures thereof. Further examples of suitable adjuvants may also be found in literature cited in WO 2007/115059 A2.

A suitable adjuvant for use according to the present invention is the adjuvant Abisco from Isconova AB, Sweden. The key components of ISCOMS are Quillaia saponins derived from the bark of the Chilean soap bark tree Quillaia saporinaria molina. Quillaia saponins are well known for their ability to activate the immune system (Ref. 32). Quillaia saponins mixed with cholesterol, and phospholipids under specific stoichiometry form spherical open cage like structures known as ISCOMS.

Another suitable adjuvant is Ginseng. Ginseng is a dry extract prepared from the root of the plant Panax ginseng, C.A. Meyer. Ginseng contains a number of active substances named ginsenosides that are a kind of saponins, chemically tri-terpenoid glycosides of the dammaran series. The ginsenosides have adjuvant properties and one of the most active adjuvant is the fraction named Rb1. It has been proved that the fraction Rb1 elicits a balanced Th1 and Th2 immune response as determined by measuring the levels of the cytokines IFN-γ, IL-2, IL-4, IL-10 secreted post vaccination with a Rb1 adjuvanted vaccine. In addition ginseng and the fraction Rb1 stimulates a strong antigen specific antibody response.

According to a suitable embodiment, the vaccine composition is a vaccine that protects susceptible mammals, suitably horses, against strangles caused by S. equi subsp. equi and against infections caused by subsp. zooepidemicus.

The vaccine composition of the present invention is provided in a physiologically administrable form. Suitably, it is administrable by intramuscular, subcutaneous, intradermal or intranasal inoculation.

Suitably, the vaccine composition of the present invention stimulates serum, mucosal and/or bronchial antibody responses directed to S. equi antigens in mammals susceptible to S. equi, suitably horses.

The present invention is also related to a method for producing an antigen or immunogen to be used in an antigenic or immunogenic composition of the present invention, which method comprises

(a) providing a DNA fragment encoding said antigen and introducing said fragment into an expression vector;

(b) introducing said vector, which contains said DNA fragment, into a compatible host cell;

(c) culturing said host cell provided in step (b) under conditions required for expression of the product encoded by said DNA fragment; and

(d) isolating the expressed product from the cultured host cell.

Preferably, said method further comprises a step (e) wherein the isolated product from step (d) is purified, e.g. by affinity chromatography or other chromatographic methods known in the art.

Accordingly, the antigens of the present invention are usually produced according to recombinant techniques.

A further embodiment of the present invention is concerned with a method for preparation of a vaccine of the present invention, which vaccine contains as immunizing component an antigenic or immunogenic composition as disclosed above, said method comprising mixing said antigenic composition and a pharmaceutically acceptable carrier.

The present invention is also related to a method for the production of an antiserum, said method comprising administering an antigenic preparation of the present invention to an animal host to produce antibodies in said animal host and recovering antiserum containing said antibodies produced in said animal host.

Moreover, the present invention is concerned with a method of prophylactic or therapeutic treatment of S. equi infection in mammals, suitably horses, comprising administering to said mammal an immunologically effective amount of a vaccine or an antiserum of the present invention.

Accordingly, the present invention is related to a method for protecting horses against S. equi infection, which method comprises inoculating a horse subcutaneously, intranasally, intradermal, orally or intramuscular, or any combination thereof with a vaccine composition of the present invention to induce an immune response against S. equi in said horse. Suitably, an immune response, in the form of IgG and/or IgA and/or IgM antibodies in the nasopharyngeal mucus, and/or serum is induced in said horse.

The present invention also relates to an antibody preparation comprising at least one, and suitably at least two, antibodies specific for a protein or a polypeptide of the present antigenic composition, which antibody/antibodies is/are polyclonal or monoclonal; or which preparation comprises a fragment of said antibodies.

The antibody preparation of the present invention could be used prophylactically or therapeutically against strangles and provides passive immunization when administered to a non-human mammal susceptible to infection by S. equi or infected by S. equi. The present invention provides a vaccine composition comprising one or several antigen components which have been prepared according to the present method using E. coli as host cells. The source of these antigens might also be the native bacteria, if methods are developed for expression and purification thereof. Alternatively, the antigens of the present invention can also be produced according to methods that are based on fusion strategies where various parts of the respective antigen are recombined resulting in a fusion protein consisting of parts from different antigens. This fusion strategy could also be suitable for introducing immune reactive part(s), e.g. T-cell epitopes or attenuated toxins (or parts thereof), thereby introducing other features suitable for optimizing the antigen presentation or localization.

The present invention can also be applied with the purpose to enhance a vaccine composition consisting of an attenuated strain of S. equi. Descriptions of such strains are e.g. disclosed in Refs. 17, 44 and WO 2009/7093014 A2. The addition of EndoSe or EndoSz or EndoS or fragments thereof to a vaccine composition (given at the same occasion or separately) of a live attenuated strain of subsp. equi should enhance the effect of immunization thereby increasing the protective effect of vaccination. In a similar way the use of EndoSz or EndoS could be applied to other vaccine formulations aiming to reduce subsp. equi, subsp. zooepidemicus and S. pyogenes infections.

The present invention may also be used in other vaccines or subunit immunogenic compositions, where the invention can be combined with one or more immunogens, antigens or epitopes selected from other pathogenic microorganisms or viruses to form multivalent subunit immunogenic compositions or vaccines. For example, concerning equine, such a multivalent subunit immunogenic composition or vaccine may comprises at least one polypeptide according to the present invention and at least one immunogen, antigen, or epitope from WEEV, EEV, VEEV, equine influenza virus, EHV-1, EHV-4, EAV, WNV, tetanus, Rhodococcus.

The present invention also provides diagnostic methods to measure antibodies against EndoSe, EndoSz and EndoS or fragments thereof. For instance, these types of methods may be used to determine antibody titers in sera before and/or after immunization or to determine antibody titers in infected mammals. The methods may also be applied to screen individual mammals to detect infected or chronical carriers of S. equi and/or S. pyogenes. Furthermore, the invention also provides a method to determine antibodies with neutralizing activity against EndoSe, EndoSz and EndoS, thereby making it possible to measure the effect of e.g. immunization procedures or to identify individuals who lack antibodies that neutralize EndoSe, EndoSz and EndoS.

### Experimental Part

### EXAMPLE 1

### Identification of EndoS Similar Proteins in subsp. equi and subsp. zooepidemicus

The DNA sequences of the genomes of S. equi subsp. equi and subsp. zooepidemicus have been determined and are available at the Sanger Centre (http://www.sangerac.uk/Projects/S_{—}equi and http://www.samerac.uk/Projects/S_{—}zooepidemicus). Using the amino acid sequence of EndoS of S. pyogenes (GenBank: AAK00850.1, SEQ ID NO: 15) the genomes of both subsp. were screened using the program BLAST (http://www.ncbi.nlm.nih.gov/BLAST/) for open reading frames coding for EndoS similar proteins. The results showed that both subsp. harbour a gene denoted endoSe (from subsp. equi) SEQ ID NO: 1 and endoSz (from subsp. zooepidemicus) SEQ ID NO: 10. The corresponding proteins are called EndoSe (SEQ ID NO: 2) and EndoSz (SEQ ID NO: 11), respectively. Sequence similarities between the EndoS, EndoSe and EndoSz proteins were studied using the ClustalW programme (http://aligngenome.jp/). The results revealed a very high degree of similarity between the proteins. Since the EndoS, EndoSe and EndoSz proteins display high similarity it is a good reason to assume that the experiments performed and the results obtained using EndoSe are also valid for EndoSz and EndoS. The cloning of the endoSe gene and expression of recombinant EndoSe and polypeptide fragments thereof are described below. The use of the EndoSe protein and fragments thereof as antigens to obtain an immunogenic response and their effects to induce protective effects and reducing severity of S. equi infection will also be described.

### EXAMPLE 2

### Constructions of E. coli Clones Harboring Various Parts of endoSe

S. equi subspecies equi strain 1866 (obtained from Nordvacc Lakemedel AB, Sweden), (WO 2004/032957 A1, Ref. 25) was used as source of DNA for cloning. Chromosomal DNA from subspecies equi strain 1866 was prepared and used as a template to amplify fragments of the endoSe gene encoding mature EndoSe (lacking the N-terminal signal sequence), hereinafter simply called EndoSe, fragment A and fragment C (the nucleotide and polypeptide sequences are presented in the sequence listing further below); SEQ ID NOS: 3, 4, 5, 6, 7, 8. To identify the predicted signal sequence, the computer program SignalP (http://www.cbs.dtu.dk/services/SignalP/) was used. The sequences of primers used to amplify the various fragments of the endoSe gene are listed in Table 1. Cleavage sites for the restriction enzymes BamHI and XhoI were included in the primer sequences to match the cloning sites in the plasmid vector pGEX-6P-1 (GE Healthcare). The PCR amplifications were performed using the primers (20 pmol/μl) and FideliTaq™ PCR Master Mix (USB Corporation, Cleveland, Ohio) using the following programme: Step 1, pre-heat 1 minute at 95° C., DNA strand separation; Step 2, 30 seconds at 95° C.; Step 3, annealing 15 seconds at 5 degrees below respective primer combination melting point; and Step 4, elongation for 2 minutes at 72° C., Steps 2-4 were run for 29 cycles. The PCR products were analysed on a 1 agarose gel, and thereafter purified using the QIAquick PCR Purification Kit™ (Qiagen). After cleavage with the restriction enzymes the fragments were purified one additional time using the same kit. After purification the respective fragment was ligated into using ReadyToGo T4DNA Ligase (GE Healthcare). After ligation, the respective sample were transformed into competent cells of E. coli strainTG1 using electroporation, and spread on LA-Amp plates (Luria-Bertani broth agar (15 g/L) plates supplemented with ampicillin, final conc. 50 μg/ml) and incubated over night at 37° C. Next day colonies were picked and cultivated and used for further experiments. To verify the presence of an insert in the respective constructs, plasmids were purified and additional PCR analyses were performed using the respective primer combination. The sequence of the respective insert was also determined by DNA sequencing using primers GexS, GexR, eq61P3 and eq61P4. Correct clones were transformed into competent cells of E. coli strain BL21 (DE3) pLys for protein expression.

TABLE 1
The primer sequences used to PCR amplify the gene endoSe, the gene
fragments A (SEQ ID NO: 5) and C (SEQ ID NO: 7) of endoSe. The
nucleotides indicated in bold correspond to introduced restriction
cleavage sites BamHI and XhoI. The primer combinations used to
amplify endoSe were Eq61p1 and Eq61p6: fragment A, Eq61p1 and
Eq61p2: fragment C, Eq61p5 and Eq61p6.

| | Primer | |
|---|---|---|
| Gene | name | Primer sequence (orientation 5′-3′). |
| endoSe (mature | Eq61p1 | GTCGGATCCGAGGATAAGGTTGTGCAAACTAG |
| endoSe) | | (SEQ ID NO: 18) |
| | Eq61p6 | GCCTCTCGAGGGATAAGCTAGTCTGTCTTTGG |
| | | (SEQ ID NO: 19) |
| endoSe | Eq61p1 | GTCGGATCCGAGGATAAGGTTGTGCAAACTAG |
| (fragment A) | | (SEQ ID NO: 18) |
| | Eq61p2 | GCAGCTCGAGTTAATATTGGGCACCGCGCTCAATC |
| | | (SEQ ID NO: 20) |
| endoSe (encoding | Eq61p5 | TGACGGATCCAAGGAGGCCAAGCTTGAAGC |
| fragment C) | | (SEQ ID NO: 21) |
| | Eq61p6 | GCCTCTCGAGGGATAAGCTAGTCTGTCTTTGG |
| | | (SEQ ID NO: 19) |
| Sequencing | Eq61p3 | TTATGGATCCGATCGCGATGGTGTAGCG |
| primer | | (SEQ ID NO: 22) |
| Sequencing | Eq61p4 | TCTTCTCGAGTTAACCGCCATGCTTAGTCACTG |
| primer | | (SEQ ID NO: 23) |
| Sequencing | GexS | GGGCTGGCAAGCCACGTTTGGTG |
| primer | | (SEQ ID NO: 24) |
| Sequencing | GexR | CCGGGAGCTGCATGTGTCAGAGG |
| primer | | (SEQ ID NO: 25) |

### EXAMPLE 3

### Purification of Mature endoSe and Parts of endoSe

The pGEX-6P-1 vector used is a part of an E. coli expression and purification system called GST-glutathione affinity system (GE Healthcare). Briefly, following the manufacturer's instructions the clones encoding mature EndoSe, fragment A and fragment C of EndoSe, respectively, were grown at 37° C. in Luria Bertani Broth medium supplemented with ampicillin (final conc. 50 μg/ml). At an optical density (OD₆₀₀)˜0.6, the growth medium was supplemented with IPTG (final conc. 0.2 mM) and the growth temperature shifted to 15° C. After incubation over night the E. coli cells were harvested and resuspended in a PBS phosphate-buffered saline [137 mM NaCl, 2.7 mM KCl, 10 mM Na₂HPO₄, 1.4 mM KH₂PO₄ (pH 7.4)] supplemented with TWEEN 20, final conc. 0.1% (v/v) (PBST) and lysozyme was added (final conc. 50 μg/ml) whereupon the cells were lysed by freezing and thawing. After centrifugation, the supernatant was sterile filtrated and batch purified with Glutathione-Sepharose beads. After extensive washing using PBST the fusion protein was treated with scissor protease to release the recombinant proteins. The eluted samples containing the antigens were dialysed against PBS. Finally, the amounts of antigens obtained were determined using spectrophotometry and the quality analyzed by SDS-PAGE (performed under reducing conditions) whereupon the gels were coomassie brilliant blue stained. The proteins were stored finally at −20° C. It should be noted that each protein produced in this system (NO: 4, NO: 6 and NO: 8) contains five additional amino acids in the N-terminal part which is derived from the vector. These amino acids are Gly-Pro-Leu-Gly-Ser. The C-terminal end of each protein is as stated since a stop codon was added in the primer sequence.

### EXAMPLE 4

### Purified Recombinant EndoSe is Enzymatically Active

It has been shown that EndoS secreted from S. pyogenes hydrolyzes the glycan on native IgG, leaving an N-acetylglucosamine with a core fucose (Ref. 8). This can be visulised by running the treated IgG on a SDS-PAGE. The heavy chain of the treated IgG is slightly smaller after treatment with EndoS.

To study the endoglycosidase activity of purified EndoSe the protein was incubated with IgG from human, mouse and horse. This was done my mixing 4 μl of IgG (1 mg/ml) with 1μ of purified EndoSe (0.2-0.7 mg/ml) for 30 minutes at 37° C. This treatment results in a heavy chain of IgG that migrates slightly faster on a SDS-PAGE. To better visulate the effect EndoSe has on IgG, human and horse IgG were cleaved with a combination of the endopeptidases IdeE and IdeE2. This treatment results in a cleavage of the heavy chain of IgG into two smaller fragments. Thus, the size differences caused by EndoSe can be seen much clearer. The cleaved human IgG was used to titrate the concentrations at which EndoSe hydrolyzes the carbohydrate residues on human IgG. The assay was performed by incubation of 4 μl cleaved human IgG (1 mg/ml) with 1μ of purified EndoSe stepwise diluted after which the mixture was incubated 30 minutes at 37° C. At theses conditions, the activity of EndoSe could be observed at a concentration as low as 2.6 μg/ml.

### EXAMPLE 5

### Presence of the Genes Similar to endoSe in S. equi subsp. zooepidemicus

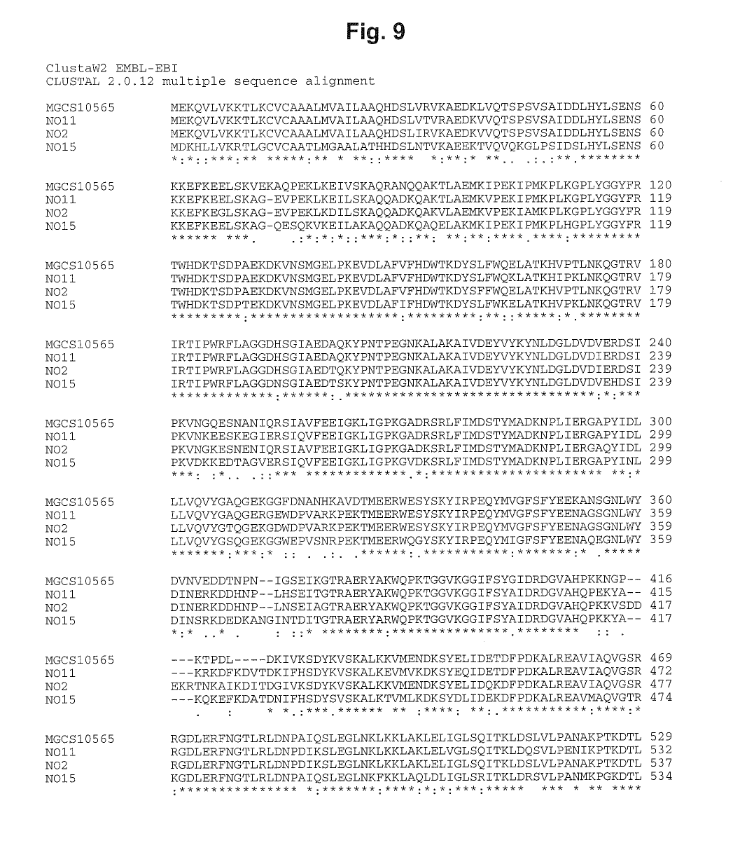

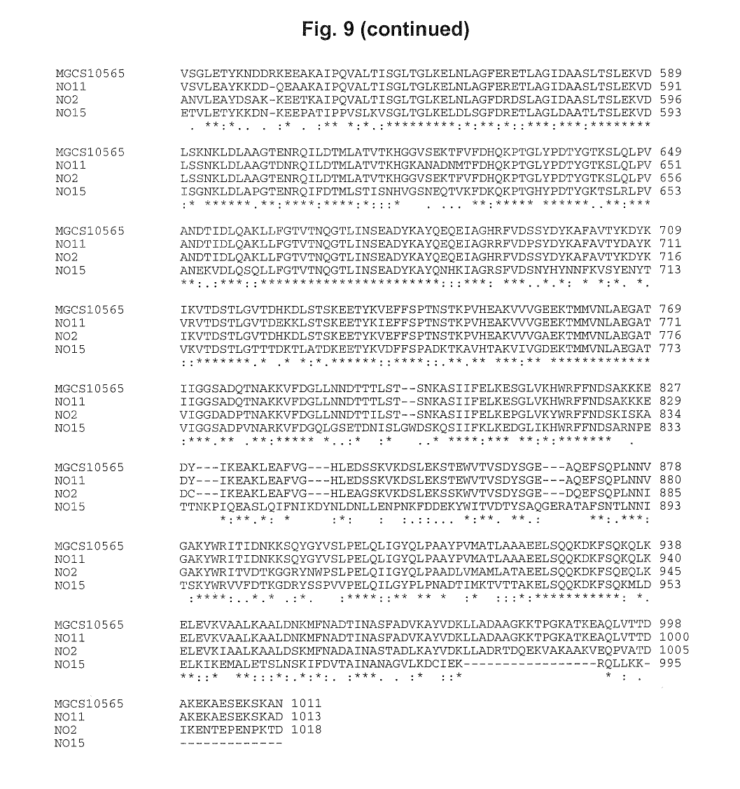

Using the S. zooepidemicus genome database (www.sanger.ac.uk/), the presence of a similar gene to endoSe, called endoSz (SEQ ID NO: 10) was identified using BLAST search. The results showed that the deduced protein called EndoSz (SEQ ID NO: 11) is highly similar to EndoSe. Further Blast2 (Swiss-Prot+TrEMBL) search using EndoSz revealed that a highly similar protein denoted Endo-beta-N-acetylglucosaminidase F2 is encoded by a human pathogenic Lancefield group C S. zooepidemicus strain MGCS10565 (Ref. Beres et al (2008) PLoS ONE 3:E3026; NCBI Reference Sequence: YP_{—}002122753.1). Further BLAST2 search using EndoS shows that a great number of strains of S. pyogenes harbour a gene encoding EndoS. Thus the presenence of endoS, endoSe or endoSz are found in various strains of S. pyogenes, subsp. equi and subsp zooepidemicus, respectively, meaning that the present invention could be applied for obtaining an immunizing composition(s) to induce protection against various infections caused by these streptococci. An example of a ClustalW2 alignment is shown in FIG. 9 revealing the high similarities between the “Endo-proteins” from various streptococci.

### EXAMPLE 6

### Preparation of Recombinant CNE

The cloning of the cne gene of S. equi supsp. equi strain 1866 and production of recombinant CNE protein (Sec 2.16 also called CNE L) has previously been reported and the production and use of recombinant CNE in vaccination trials is disclosed in WO 2004/032957 A1, WO 2009/075646 A1, Ref. 25. In the present invention the recombinant CNE protein used in vaccination trials was Sec 2.16. The GenBank accession number of the cne gene is AY193773.

### EXAMPLE 7

### Immunisation of Mice with CNE, EndoSe+CNE or EndoSe

When mice were immunized with EndoSe and subsequently infected with S. equi subsp. zooepidemicus or S. pyogenes, fifteen animals were used in each group. Mice (NMRI) weighing approximately 23-25 g were kept in cages of five animals in each. The mice were immunised intranasally with 12 micrograms of each antigen and 10 microgram of Abisco 300 (Isconova AB, Sweden). Ten animals were immunised with CNE, 10 animals were immunised with EndoSe and CNE together and 10 were given Abisco 300 adjuvant only to serve as a negative control. The total volume was kept to less than 24 μl and applied into the nostrils twice with 30 minutes interval of mice anaesthetized with Isoflovet (Abbot Laboratories, England). Immunisations were given on days 0, 14 and 21.

### EXAMPLE 8

### Experimental Infection with Streptococcus equi subsp. equi

Experimental infection was given on day 28 (7 days after last time of immunisation). S. equi subsp. equi strain 1866 from a clinical case of strangles was used. The strain was first passed through an animal by inoculating ca 10⁶ CFU into the nostrils of an anaesthetized mouse. Bacteria were recovered after 7 days from the nose of the mouse and grown on BG plates (agar plates containing 5% sheep blood 0.01% gentiana violet) at 37° C. in 5% CO₂. A single colony was grown on BG plates overnight at 37° C. and resuspended in Todd Hewitt Broth (Oxoid, Basingstoke, Hampshire, United Kingdom) (THB) with 1% yeast extract (THY). The bacteria were kept at −80° C. in vials and a new vial was used for each experiment. To infect mice, bacteria were grown on BG plates at 37° C. in 5% CO₂ overnight, followed by inoculation into THB supplemented with 1% Yeast extract (THY) and grown without shaking over night. The culture was then diluted 10 times into THY and 10% horse serum (Sigma) and grown for 4 hours at 37° C. in 5% CO₂. The culture was centrifuged and resuspended in THB. A dose containing 1×10⁶ CFU in 10 μl was used for all S. equi infections of mice. The animals were followed daily. Bacterial nasal growth was scored on a four-graded scale from 0 to +++ by gently pressing the nose of the animal onto a BG plate in a reproducible manner. The nasal sample was then spread out onto the entire surface of the plate. One + means 5-100 colonies; two + means more than 100 and three + means confluent growth. The weight was determined every day and the percentage of weight-loss was calculated.

Experimental infections were also performed following exactly the same procedures as described above for S. equi but with either Streptococcus zooepidemidus (strain 1577, ST88) or Streptococcus pyogenes (strain MGAS 5005). However, the inoculation doses in the mice differed. For S. zooepidemicus 9×10⁶ CFU and for S. pyogenes 8×10⁷ CFU were given in volumes of 10 μl the nostrils.

### EXAMPLE 9

### Experimental Results of Vaccination with CNE, CNE+EndoSe or EndoSe

Three groups of mice (n=3×10) were immunised with 1) CNE (SEQ ID NO: 9), 2) EndoSe (SEQ ID NO: 4) +CNE, and 3) non-immunised group where the antigen was replaced with PBS, but still containing the adjuvant.

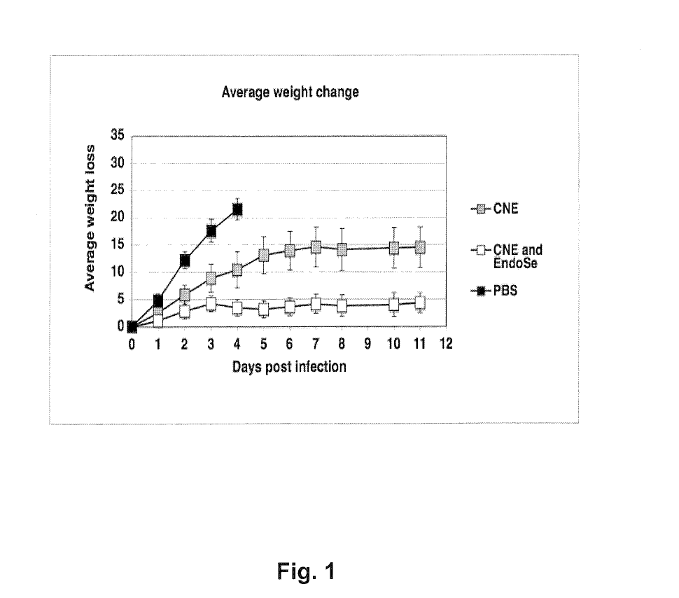

A typical sign of infection in mice infected with S. equi subsp. equi is the loss of weight. The percentage weight loss over time was thus determined. FIG. 1 shows that animals vaccinated with CNE were protected from infection, reflected by a milder loss of weight compared with control animals; e.g. p-values=0.017, 0.016, 0.009, and 0.050 for days 2, 3, 4, and 5 respectively (student's t-test). However, the addition of EndoSe to CNE improves the protection resulting in even lower p-values; p-values=0.0003 for day 2 and <0.0001 for all days during the period 3 to 11 days when comparing with the non-vaccinated control group. The improved protection resulting from adding EndoSe to CNE was significant; e.g. p-values=0.07, 0.015, 0.017, and 0.018 for days 4, 5, 6, and 7, respectively, when comparing the CNE with the CNE+EndoSe groups.

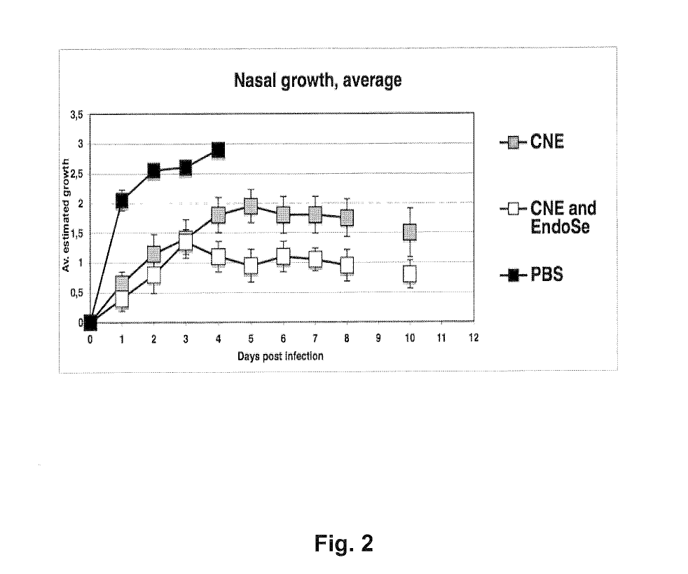

Another sign of persistent infection of mice with S. equi subsp. equi is the colonisation of bacteria in the upper respiratory airways. Nasal growth of S. equi was therefore determined daily on a four graded scale. FIG. 2 shows that after 3 to 4 days, the non-vaccinated control animals were heavily colonized with bacteria. Mice vaccinated with CNE were significantly less colonized compared with the control group. The frequency of animals grossly colonized nasally with bacteria (scoring 2-3) was significantly different between the two groups. P-values=0.005, 0.003, 0.011, 0.011, 0.08, and 0.011 for days 1, 2, 3, 4, 5, and 6, respectively (Fisher's exact test). As shown in FIG. 2, addition of EndoSe to CNE in the vaccine further reduced colonization, resulting in even lower p-values when compared with the non-vaccinated control group. P=0.005, 0.003, 0.0007, 0.0007, 0.0007, and 0.003 for days 1, 2, 3, 4, 5, and 6, respectively.

Conclusions: Immunization with CNE alone induces a significant protection in vaccinated animals. Immunization with EndoSe+CNE induces a significant elevated protection in vaccinated animals.

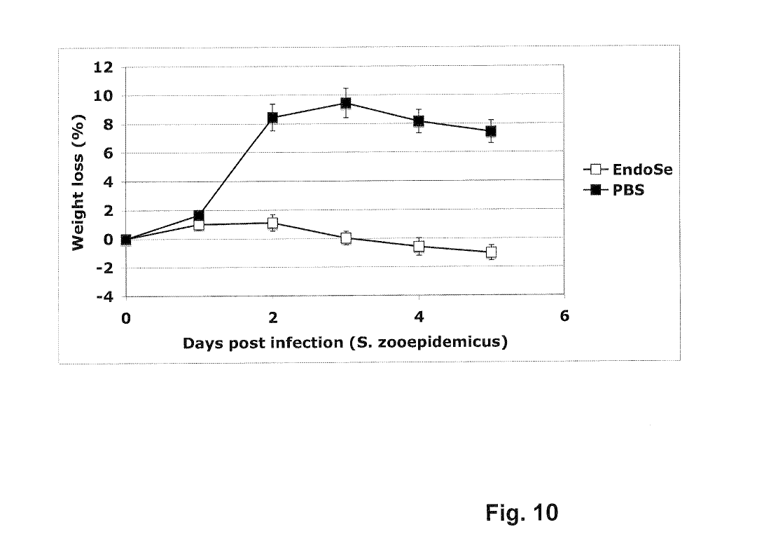

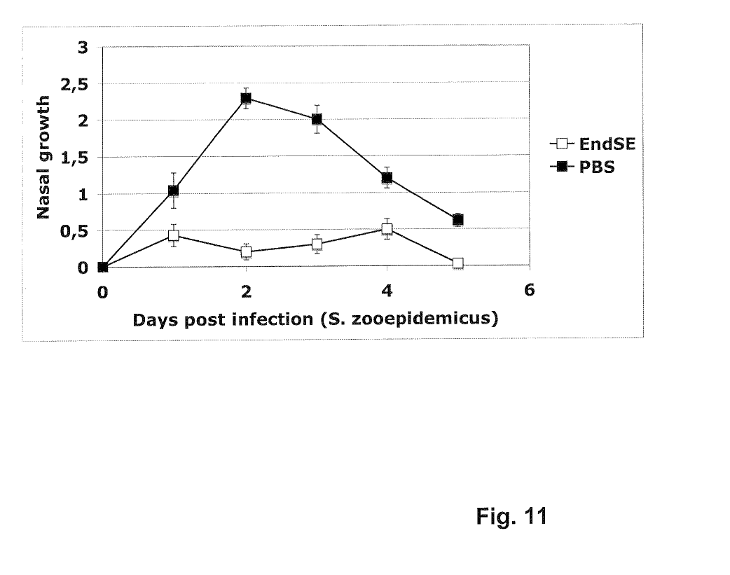

Mice (n=15), which had been vaccinated with EndoSe, and non-immunized mice (n=15) were also infected with S. equi subsp. zooepidemicus. Nasal colonization and weight loss were followed daily. Mice infected with S. equi subsp. zooepidemicus became infected but to a milder extent than those infected with S. equi subsp. equi and all mice recovered from infection. Nasal colonisation was transient and weight loss reversible. However, the group vaccinated with EndoSe was even less affected than non-vaccinated and never lost weight (p<0.0001 for days 2 to 5) as shown in FIG. 10. Nasal colonisation was minimal (p<0.0002 for days 2 and 3) compared to the non-vaccinated control group (FIG. 11).

Conclusion: Immunisation with EndoSe protects against infections caused by S. equi subsp. zooepidemicus.

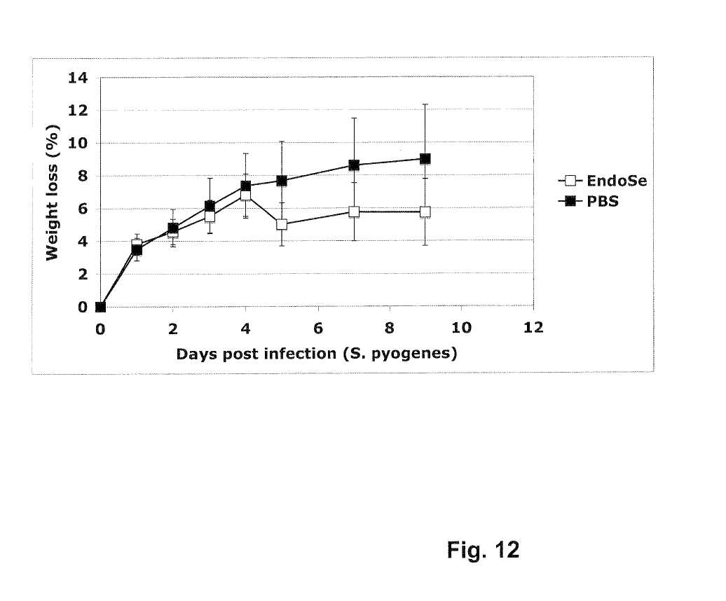

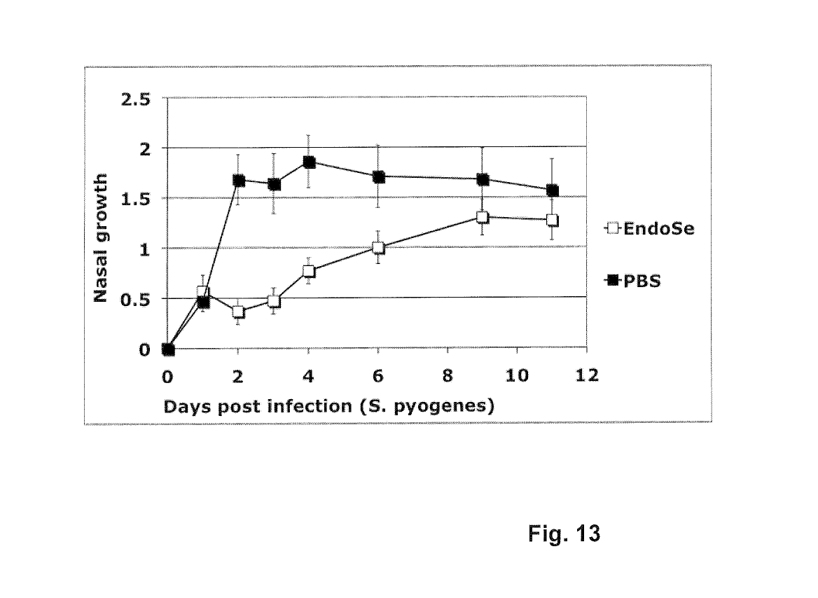

Mice (n=15), which had been vaccinated with EndoSe, and non-immunized mice (n=15) were also infected with S. pyogenes. Infection with S. pyogenes resulted in a more slow progress of infection. The mice vaccinated with EndoSe did not differ significantly in weight loss from the non-vaccinated group (FIG. 12). However, nasal colonisation was significantly lower in the vaccinated group (p=0.006, 0.002, 0.005, 0.002, 0.008 for days 1 to 5, respectively) (FIG. 13). The nasal colonisation in the non-vaccinated group did not seem to lead to illness resulting in weight loss.

Conclusion: Immunisation with EndoSe protects against infections caused by S. pyogenes.

### EXAMPLE 10

### Determination of Antibody Levels in Immunized Mice

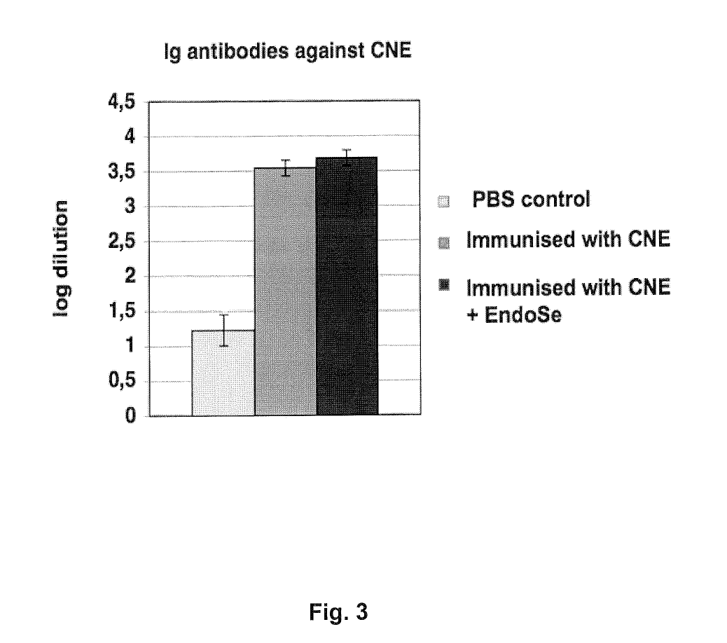

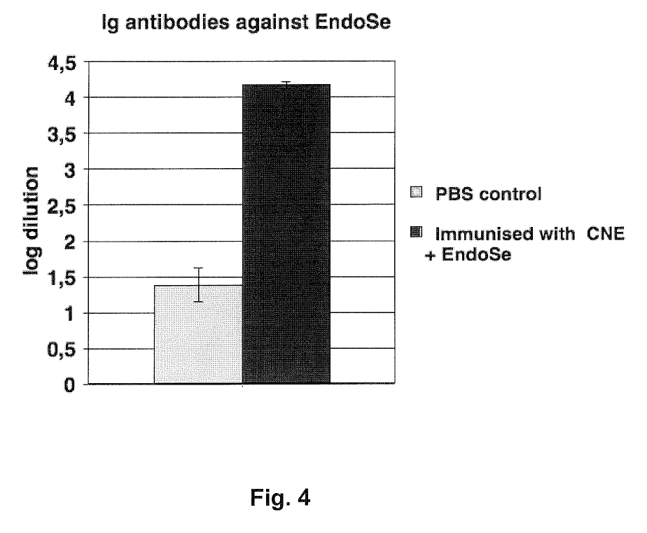

Mice were immunized as described above. Serum samples were collected 5 days after last vaccination. Standard Enzyme Linked Immuno Sorbent Assay (ELISA) was used to determine levels of IgG specifically directed against CNE and EndoSe. Briefly, microtiter plates were coated with 100 μl over night at room temperature with either protein (CNE or EndoSe) at 4 μg/ml in Phosphate Buffered Saline (PBS). Bovine Serum Albumin, 100 μl at 2%, was added (1 hour at 37° C.). The plates were washed with PBS with 0.05% Tween (PBST). Serum samples were added at serial dilutions, starting at a 40-fold dilution (1 hour at 37° C.) followed by washing. The specific binding of IgG to the antigens was monitored by adding anti mouse IgG antibodies raised in rabbit conjugated with Horse Radish Peroxidase (Sigma Chemical Co, Mo, USA); 100 μl per well at 1000-fold dilution. After washing in PBST, binding of the conjugate was measured by adding OPD substrate according to the instructions provided by the manufacturer (Dako, Glostrup, Denmark). The coloration was determined at 492 nm in a standard ELISA spectrophotometer. The obtained absorbance values were plotted as a function of serum dilution. For each sample, the 10log values of the dilution required to bring down the absorbance value to 1.5 were determined. I.e., if a sample requires a 2000 fold dilution to give an absorbance of 1.5, a value of 3.30 is assigned to that sample. FIG. 3 shows antibody titers against CNE in mice immunized with CNE or with CNE+EndoSe. FIG. 4 shows antibody titers against EndoSe in mice immunized with CNE+EndoSe. Both Figures also show antibody levels in control mice.

Conclusions: Immunization with EndoSe together with another antigen, here exemplified with CNE, does not reduce antibody response against the co-administered antigen. Immunization with EndoSe together with CNE results in a strong IgG response against both antigens.

### EXAMPLE 11

### Immunisation of Mice with EndoSe Fragments A and C

Mice were immunised with EndoSe fragments A (SEQ ID NO: 6) and fragment C (SEQ ID NO: 8) in separate groups, ten in each group, as described for CNE and EndoSe+CNE. A control group of ten mice were given adjuvant only.

### EXAMPLE 12

### Experimental Results of Vaccination with EndoSe Fragments A and C

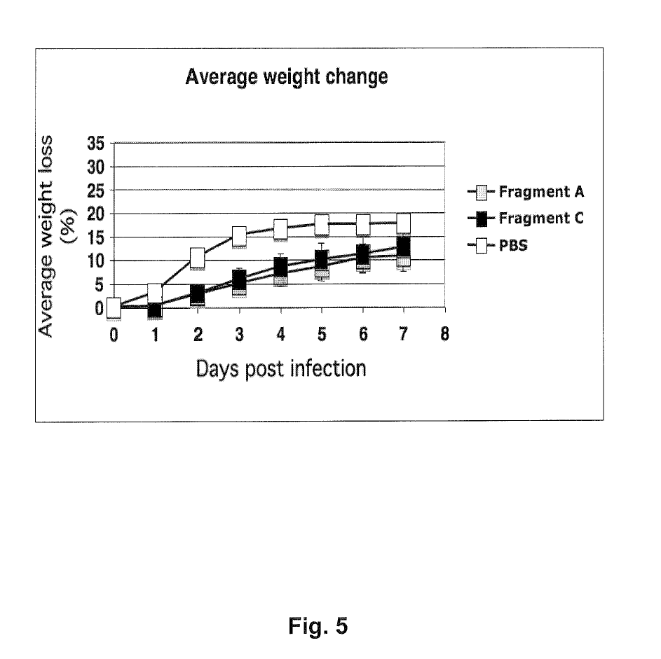

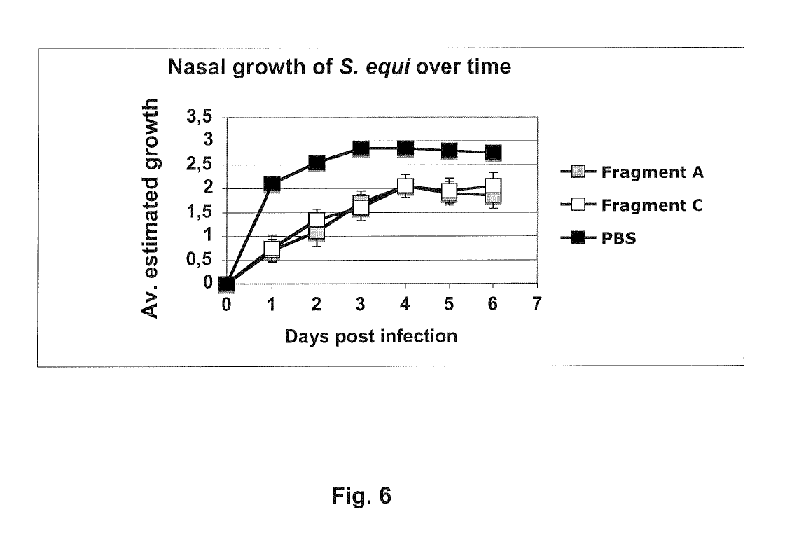

Three groups of mice (n=3×10) were vaccinated with fragment A, fragment C or adjuvant only. The mice were experimentally infected as described in Example 8. As shown in FIG. 5, average weight losses of the vaccinated mice were significantly less than in non-vaccinated control mice. Comparing mice immunised with fragment A with control mice gave p-values of 0.02, 0.001, 0.004, 0.01, and 0.02 for days 1 to 5, respectively. Comparing mice immunised with fragment C with control mice gave p-values of 0.05, 0.006, 0.009, 0.03, and 0.08 for days 1 to 5, respectively (t-test). FIG. 6 shows average estimated nasal growth of bacteria. For several of the observations, differences are significant (Fischer's exact test); for fragment A vs. control, p=0.02, 0.08, 0.08, and 0.08 for days 2-5, respectively. For fragment C vs. control, p=0.02, 0.01, 0.03, and 0.01 for days 2-5, respectively.

Conclusion: Immunization with fragments of EndoSe in this example exemplified with fragment A and C, respectively induces a significant protection in vaccinated animals. This implies that also fragments of other EndoSe like proteins, e.g. EndoSz and EndoS could be used.

### EXAMPLE 13

### Immunisation of Mice with EndoSe, Eq5 or EndoSe and Eq5

Mice were immunised with EndoSe, Eq5 or EndoSe and Eq5 in separate groups, 8 in each group, as described for CNE and EndoSe+CNE (Example 7). A control group of 8 mice were given adjuvant only.

### EXAMPLE 14

### Experimental Results of Vaccination with EndoSe, Eq5 or EndoSe and Eq5

Four groups of mice (n=4×8) were immunised with 1) EndoSe (SEQ ID NO. 4 produced according to Example 3), 2) Eq5 (WO 2009/075646 A1, SEQ ID NO: 3), 3) EndoSe+Eq5, and 4) non-immunised group where the antigen was replaced with PBS, but still containing the adjuvant.

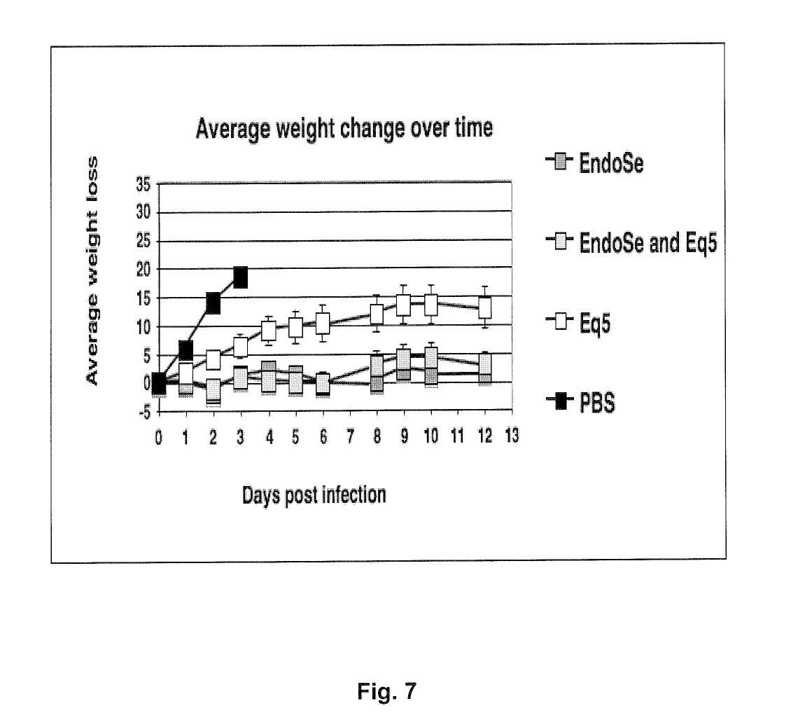

A typical sign of infection in mice infected with S. equi subsp. equi is the loss of weight. The percentage weight loss over time was thus determined. FIG. 7 shows that animals vaccinated with EndoSe or vaccinated with EndoSe+Eq5 were protected from infection, reflected by a milder loss of weight compared with control animals; e.g. p-values<0.0001 for days 1-12 for both groups vs. the control group. (student's t-test).

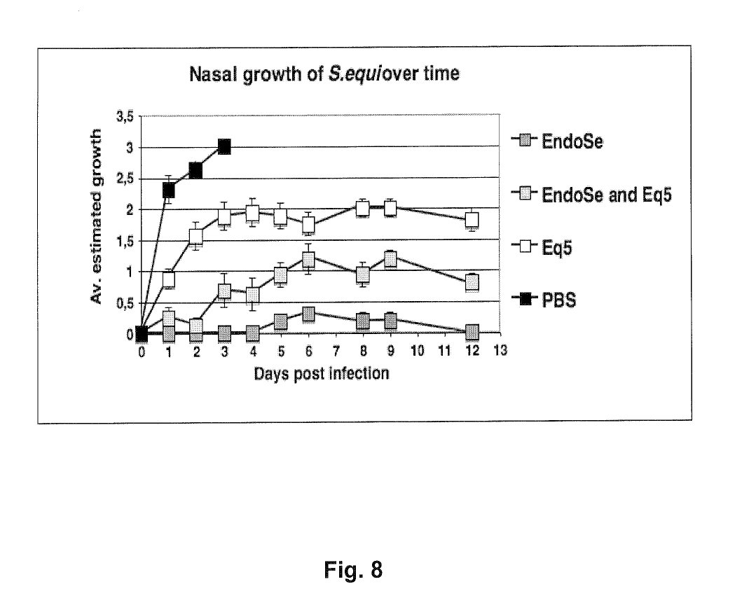

Another sign of persistent infection of mice with S. equi subsp. equi is the colonisation of bacteria in the upper respiratory airways. Nasal growth of S. equi was therefore determined daily on a four graded scale. FIG. 8 shows that after 3 to 4 days, the non-vaccinated control animals were heavily colonized with bacteria. Mice vaccinated with EndoSe or EndoSe+Eq5 were significantly less colonized compared with the control group. The frequency of animals grossly colonized nasally with bacteria (scoring 2-3) was significantly different between the two groups. P-values<0.001 for days 2-12 for both groups vs. the control group (Fisher's exact test).

Conclusion: Immunization with full-length EndoSe alone or in combination with another antigen (here exemplified by Eq5) induces a significant protection in vaccinated animals. This implies that also other EndoSe like proteins, e.g. EndoSz and EndoS, could be used. Furthermore, immunization with full-length EndoSe and another antigen, in this example exemplified by Eq5, does not reduce the immunizing effect of EndoSe.

### EXAMPLE 15

### Antisera Against EndoSe Inhibits Enzymatic Activity of EndoSe

Sera from mice immunized with EndoSe+CNE (see Examples 7-10) were collected from 5 mice 11 days after challenge with S. equi (these mice's had no symptom of infection). The sera were pooled and diluted in steps of two and used to investigate the presences of antibodies that inhibited the activity of EndoSe. The assay was performed similarly as described in Example 4. Cleaved human IgG (1 mg/ml) and purified EndoSe (10 μg/ml) was used in combination with diluted antisera. Briefly, 1 μl of EndoSe was mixed with 1 μl diluted antisera and incubated for 5 min at room temperature. Thereafter, 4 μl IgG were added and the mixture was incubated for 30 min at 37° C. after which the mixture was analysed by SDS-PAGE. The sera could be diluted 32 times and still inhibit the activity of EndoSe. Sera from mice immunized with EndoA and EndoC were also collected and tested for inhibitory activity of the enzymatic activity of EndoSe. However, no inhibitory effect was observed.

Conclusions: Immunization of a mammal using EndoSe provokes an immune response generating antibodies that inhibit the enzymatic activity of EndoSe.

### EXAMPLE 16

### Antisera Against EndoSe Inhibits Enzymatic Activity of EndoSe, Additional Experimental Demonstration

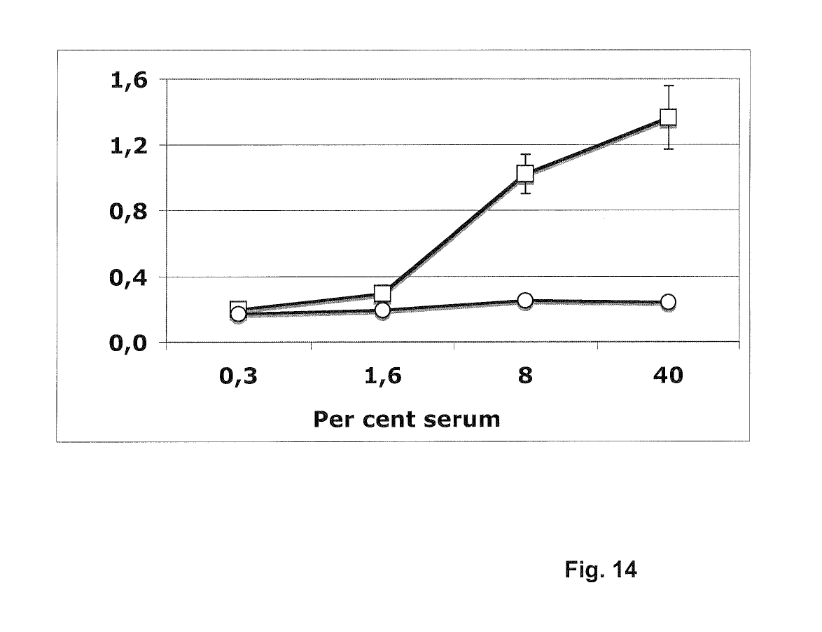

Microtiter wells (Nunc) were coated with EndoSe at 10 μg/ml. Free sites were blocked by adding 2% BSA for 1 hour at 37° C. Horse IgG (Jackson Immuno Research Laboratories) (at 10 μg/ml) was, after washing, added to the immobilised EndoSe and its binding was determined with HRP conjugated anti Horse IgG (Sigma) followed by development with OPD substrate (Dako) and measurement spectrophotometrically at 492 nm. IgG can bind to immobilised EndoSe in microtitre wells. However, when IgG (1 mg/ml) was treated with soluble EndoSe (6 μg/ml) prior to addition to the stationary phase EndoSe (coating concentration 10 μg/ml), binding was completely eliminated. Therefore, interaction between the two molecules appears only to take place at the catalytic site on IgG. In the next step, the soluble EndoSe (30 μg/ml) was pre-treated with various amounts, as indicated in FIG. 14, of anti serum against EndoSe, raised in mice as described elsewhere. The ability of EndoSe to damage the binding ability of IgG to stationary EndoSe was then inhibited, as shown in FIG. 14. A higher concentration of anti EndoSe serum (squares) inhibits the ability of EndoSe to prevent IgG from binding to immobilised EndoSe, thus a restored binding of IgG was obtained. Negative serum (circles) had no such effect. Mean values and SE from sera from six immunised mice and from three negative mice are shown in FIG. 14.

Overall conclusions from immunization experiments: Immunization with EndoSe or fragments thereof does not result in any obvious clinical side effects observed in the immunized animals. Thus immunization with EndoSe or fragments thereof seems to be safe. Furthermore immunizations with EndoSe or protein fragments thereof (herein exemplified by fragment A and C, respectively) induce protection in a mammalian species (herein exemplified by using mice) against streptococcal infections, herein exemplified by using S. equi subsp. equi, S. equi subsp. zooepidemicus and S. pyogenes. However, the protective effect of immunization was much higher using mature EndoSe than the fragments A and C. Furthermore, immunization of a mammal using EndoSe results in antibodies that inhibit the enzymatic activity of EndoSe.

A number of literature references, patents and patent applications have been referred to in the description above. The full disclosures of these literature references, patents and patent applications are incorporated herein by reference. Further, the present invention is not limited to the above-described preferred embodiments. Various alternatives, modifications and equivalents may be used. Therefore, the above embodiments should not be taken as limiting the scope of the invention, which is defined by the appending claims.

SEQUENCE LISTING

Nucleotide sequence of endoSe.
SEQ ID NO: 1
ATGGAAAAACAGGTGTTAGTCAAGAAAACACTGAAAT
GTGTTTGTGCTGCAGCGTTAATGGTGGCTATATTAGCTGCCCAACATGATTCACTCATAA
GGGTCAAGGCAGAGGATAAGGTTGTGCAAACTAGTCCATCAGTCTCTGCTATTGATGACC
TACATTACCTGTCGGAAAACAGTAAAAAAGAATTTAAGGAGGGGTTATCAAAGGCAGGAG
AAGTACCTGAAAAGCTAAAGGATATTTTATCCAAGGCACAGCAGGCAGATAAGCAGGCAA
AGGTTCTTGCAGAAATGAAGGTTCCTGAAAAAATAGCCATGAAGCCTTTAAAGGGGCCTC
TTTATGGTGGCTATTTTAGGACTTGGCATGATAAAACATCAGATCCGGCTGAAAAGGATA
AGGTTAATTCTATGGGAGAATTGCCTAAGGAGGTTGACTTAGCCTTTGTTTTCCATGATT
GGACCAAGGATTATAGCTTTTTCTGGCAAGAATTGGCGACCAAGCATGTGCCAACGCTGA
ACAAGCAGGGAACACGTGTGATTCGTACCATTCCATGGCGGTTCCTTGCAGGCGGTGATC
ATAGTGGTATTGCTGAAGATACGCAAAAATACCCAAATACTCCAGAGGGAAATAAGGCCT
TGGCAAAGGCTATTGTAGATGAATACGTTTATAAATATAATCTTGATGGTTTAGATGTTG
ATATTGAGCGGGATAGCATTCCAAAAGTAAATGGAAAAGAGAGTAACGAAAATATTCAGC
GCTCTATTGCTGTTTTTGAAGAAATTGGCAAGCTTATTGGGCCAAAGGGCGCTGACAAGT
CACGTTTGTTCATTATGGATAGCACCTACATGGCTGACAAGAACCCATTGATTGAGCGCG
GTGCCCAATATATTGATTTGCTGCTTGTGCAGGTTTATGGCACTCAAGGTGAGAAGGGAG
ATTGGGATCCAGTCGCTAGAAAACCTGAAAAGACAATGGAGGAACGTTGGGAATCGTATA
GCAAATACATTCGTCCTGAGCAGTACATGGTTGGTTTTTCTTTCTATGAGGAAAATGCGG
GCAGTGGTAACCTCTGGTATGATATTAATGAGAGGAAAGATGATCATAATCCGTTAAATT
CAGAGATAGCTGGTACTCGTGCTGAGCGTTATGCAAAATGGCAGCCTAAGACAGGTGGTG
TCAAGGGAGGGATTTTCTCTTATGCGATTGATCGCGATGGTGTAGCGCATCAACCTAAAA
AAGTCTCAGATGATGAGAAAAGAACTAACAAGGCTATAAAGGATATAACAGATGGTATTG
TCAAATCAGATTATAAGGTTTCTAAGGCCTTGAAGAAGGTTATGGAAAATGACAAATCCT
ATGAGCTGATTGATCAGAAAGATTTTCCAGACAAGGCTTTGCGAGAAGCAGTTATTGCAC
AGGTTGGAAGCAGAAGAGGGGATTTAGAGCGGTTCAATGGAACCCTGCGCTTAGACAATC
CGGATATCAAGAGTTTAGAAGGCCTGAATAAGCTTAAAAAACTAGCTAAGCTAGAGCTAA
TCGGTCTATCACAAATCACAAAGCTGGATAGCTTAGTCCTACCTGCAAATGCTAAGCCGA
CCAAGGATACGCTGGCCAATGTTCTTGAAGCCTACGACAGCGCTAAGAAGGAAGAGACTA
AGGCGATTCCACAGGTGGCTCTGACCATTTCTGGTCTAACTGGCTTGAAGGAATTAAATC
TTGCTGGCTTTGATCGTGATAGCTTGGCTGGAATTGACGCAGCTAGCCTAACCTCTCTTG
AAAAGGTGGATCTCTCTAGTAATAAGCTGGACTTAGCAGCTGGTACGGAAAATCGTCAGA
TTCTTGATACCATGCTGGCAACAGTGACTAAGCATGGCGGTGTTAGCGAAAAGACGTTTG
TATTTGATCATCAAAAGCCTACTGGTCTTTATCCTGATACTTATGGCACTAAGAGCCTTC
AGTTACCAGTAGCAAATGATACAATTGATTTGCAGGCTAAGCTTTTATTTGGAACAGTTA
CCAATCAGGGCACGCTAATCAATAGCGAAGCTGACTATAAGGCTTATCAGGAGCAGGAAA
TAGCAGGTCACCGTTTTGTTGATTCAAGCTATGATTACAAAGCCTTTGCAGTGACCTACA
AGGACTATAAGATCAAGGTGACTGACTCAACCTTAGGTGTCACTGATCACAAGGACTTAT
CCACTAGCAAGGAGGAGACCTACAAGGTTGAATTCTTTAGCCCTACTAATAGCACTAAGC
CTGTGCATGAGGCTAAGGTTGTCGTTGGTGCGGAAAAAACCATGATGGTTAACCTAGCAG
AGGGAGCAACTGTGATTGGTGGTGATGCAGATCCAACAAATGCAAAAAAAGTGTTTGATG
GTTTGCTCAATAATGATACAACAATTCTGTCAACTAGCAATAAAGCTTCTATCATTTTTG
AACTTAAAGAGCCTGGCTTAGTCAAGTATTGGCGTTTCTTTAATGACAGCAAAATTAGTA
AAGCTGACTGTATTAAGGAGGCCAAGCTTGAAGCCTTTGTTGGCCATCTTGAAGCTGGCT
CAAAGGTAAAGGATAGCTTGGAAAAATCATCAAAATGGGTAACAGTTTCAGATTATTCAG
GAGAGGACCAAGAGTTTAGCCAGCCGTTAAACAACATTGGTGCCAAATATTGGAGAATAA
CAGTTGATACTAAGGGAGGACGTTACAATTGGCCATCACTTCCTGAGCTTCAAATCATTG
GTTATCAATTACCGGCTGCGGATCTTGTGATGGCAATGCTAGCTACTGCAGAGGAGCTAT
CTCAGCAAAAAGACAAGTTCTCTCAAGAGCAGCTTAAGGAGCTCGAAGTCAAAATAGCTG
CCTTAAAGGCTGCTTTAGATAGTAAGATGTTTAATGCCGATGCTATTAACGCTAGTACTG
CTGATCTGAAGGCTTATGTTGATAAGCTTTTAGCTGATAGAACTGATCAGGAAAAAGTAG
CTAAAGCAGCTAAAGTTGAGCAGCCTGTGGCTACTGACATAAAAGAAAATACTGAGCCAG
AAAATCCAAAGACAGAC.
Amino acid sequence of EndoSe.
SEQ ID NO: 2
MEKQVLVKKTLKCVCAAALMVAILAAQHDSLIRVKAEDKVVQTSPSVSAIDDLHYL
SENSKKEFKEGLSKAGEVPEKLKDILSKAQQADKQAKVLAEMKVPEKIAMKPLKGPLYGG
YFRTWHDKTSDPAEKDKVNSMGELPKEVDLAFVFHDWTKDYSFEWQELATKHVPTLNKQG
TRVIRTIPWRFLAGGDHSGIAEDTQKYPNTPEGNKALAKAIVDEYVYKYNLDGLDVDIER
DSIPKVNGKESNENIQRSIAVFEEIGKLIGPKGADKSRLFIMDSTYMADKNPLIERGAQY
IDLLLVQVYGTQGEKGDWDPVARKPEKTMEERWESYSKYIRPEQYMVGFSFYEENAGSGN
LWYDINERKDDHNPLNSEIAGTRAERYAKWQPKTGGVKGGIFSYAIDRDGVAHQPKKVSD
DEKRTNKAIKDITDGIVKSDYKVSKALKKVMENDKSYELIDQKDFPDKALREAVIAQVGS
RRGDLERFNGTLRLDNPDIKSLEGLNKLKKLAKLELIGLSQITKLDSLVLPANAKPTKDT
LANVLEAYDSAKKEETKAIPQVALTISGLTGLKELNLAGFDRDSLAGIDAASLTSLEKVD
LSSNKLDLAAGTENRQILDTMLATVTKHGGVSEKTFVFDHQKPTGLYPDTYGTKSLQLPV
ANDTIDLQAKLLFGTVTNQGTLINSEADYKAYQEQEIAGHRFVDSSYDYKAFAVTYKDYK
IKVTDSTLGVTDHKDLSTSKEETYKVEFFSPTNSTKPVHEAKVVVGAEKTMMVNLAEGAT
VIGGDADPTNAKKVFDGLLNNDTTILSTSNKASIIFELKEPGLVKYWRFFNDSKISKADC
IKEAKLEAEVGHLEAGSKVKDSLEKSSKWVTVSDYSGEDQEFSQPLNNIGAKYWRITVDT
KGGRYNWPSLPELQIIGYQLPAADLVMAMLATAEELSQQKDKFSQEQLKELEVKIAALKA
ALDSKMFNADAINASTADLKAYVDKLLADRTDQEKVAKAAKVEQPVATDIKENTEPENPK
TD.
Nucleotide sequence of endoSe encoding mature EndoSe.
SEQ ID NO: 3
GAGGATAAGGTTGTGCAAACTAGTCCATCAGTCTCTGCTATTGATGACC
TACATTACCTGTCGGAAAACAGTAAAAAAGAATTTAAGGAGGGGTTATCAAAGGCAGGAG
AAGTACCTGAAAAGCTAAAGGATATTTTATCCAAGGCACAGCAGGCAGATAAGCAGGCAA
AGGTTCTTGCAGAAATGAAGGTTCCTGAAAAAATAGCCATGAAGCCTTTAAAGGGGCCTC
TTTATGGTGGCTATTTTAGGACTTGGCATGATAAAACATCAGATCCGGCTGAAAAGGATA
AGGTTAATTCTATGGGAGAATTGCCTAAGGAGGTTGACTTAGCCTTTGTTTTCCATGATT
GGACCAAGGATTATAGCTTTTTCTGGCAAGAATTGGCGACCAAGCATGTGCCAACGCTGA
ACAAGCAGGGAACACGTGTGATTCGTACCATTCCATGGCGGTTCCTTGCAGGCGGTGATC
ATAGTGGTATTGCTGAAGATACGCAAAAATACCCAAATACTCCAGAGGGAAATAAGGCCT
TGGCAAAGGCTATTGTAGATGAATACGTTTATAAATATAATCTTGATGGTTTAGATGTTG
ATATTGAGCGGGATAGCATTCCAAAAGTAAATGGAAAAGAGAGTAACGAAAATATTCAGC
GCTCTATTGCTGTTTTTGAAGAAATTGGCAAGCTTATTGGGCCAAAGGGCGCTGACAAGT
CACGTTTGTTCATTATGGATAGCACCTACATGGCTGACAAGAACCCATTGATTGAGCGCG
GTGCCCAATATATTGATTTGCTGCTTGTGCAGGTTTATGGCACTCAAGGTGAGAAGGGAG
ATTGGGATCCAGTCGCTAGAAAACCTGAAAAGACAATGGAGGAACGTTGGGAATCGTATA
GCAAATACATTCGTCCTGAGCAGTACATGGTTGGTTTTTCTTTCTATGAGGAATATGCGG
GCAGTGGTAACCTCTGGTATGATATTAATGAGAGGAAAGATGATCATAATCCGTTAAATT
CAGAGATAGCTGGTACTCGTGCTGAGCGTTATGCAAAATGGCAGCCTAAGACAGGTGGTG
TCAAGGGAGGGATTTTCTCTTATGCGATTGATCGCGATGGTGTAGCGCATCAACCTAAAA
AAGTCTCAGATGATGAGAAAAGAACTAACAAGGCTATAAAGGATATAACAGATGGTATTG
TCAAATCAGATTATAAGGTTTCTAAGGCCTTGAAGAAGGTTATGGAAAATGACAAATCCT
ATGAGCTGATTGATCAGAAAGATTTTCCAGACAAGGCTTTGCGAGAAGCAGTTATTGCAC
AGGTTGGAAGCAGAAGAGGGGATTTAGAGCGGTTCAATGGAACCCTGCGCTTAGACAATC
CGGATATCAAGAGTTTAGAAGGCCTGAATAAGCTTAAAAAACTAGCTAAGCTAGAGCTAA
TCGGTCTATCACAAATCACAAAGCTGGATAGCTTAGTCCTACCTGCAAATGCTAAGCCGA
CCAAGGATACGCTGGCCAATGTTCTTGAAGCCTACGACAGCGCTAAGAAGGAAGAGACTA
AGGCGATTCCACAGGTGGCTCTGACCATTTCTGGTCTAACTGGCTTGAAGGAATTAAATC
TTGCTGGCTTTGATCGTGATAGCTTGGCTGGAATTGACGCAGCTAGCCTAACCTCTCTTG
AAAAGGTGGATCTCTCTAGTAATAAGCTGGACTTAGCAGCTGGTACGGAAAATCGTCAGA
TTCTTGATACCATGCTGGCAACAGTGACTAAGCATGGCGGTGTTAGCGAAAAGACGTTTG
TATTTGATCATCAAAAGCCTACTGGTCTTTATCCTGATACTTATGGCACTAAGAGCCTTC
AGTTACCAGTAGCAAATGATACAATTGATTTGCAGGCTAAGCTTTTATTTGGAACAGTTA
CCAATCAGGGCACGCTAATCAATAGCGAAGCTGACTATAAGGCTTATCAGGAGCAGGAAA
TAGCAGGTCACCGTTTTGTTGATTCAAGCTATGATTACAAAGCCTTTGCAGTGACCTACA
AGGACTATAAGATCAAGGTGACTGACTCAACCTTAGGTGTCACTGATCACAAGGACTTAT
CCACTAGCAAGGAGGAGACCTACAAGGTTGAATTCTTTAGCCCTACTAATAGCACTAAGC
CTGTGCATGAGGCTAAGGTTGTCGTTGGTGCGGAAAAAACCATGATGGTTAACCTAGCAG
AGGGAGCAACTGTGATTGGTGGTGATGCAGATCCAACAAATGCAAAAAAAGTGTTTGATG
GTTTGCTCAATAATGATACAACAATTCTGTCAACTAGCAATAAAGCTTCTATCATTTTTG
AACTTAAAGAGCCTGGCTTAGTCAAGTATTGGCGTTTCTTTAATGACAGCAAAATTAGTA
AAGCTGACTGTATTAAGGAGGCCAAGCTTGAAGCCTTTGTTGGCCATCTTGAAGCTGGCT
CAAAGGTAAAGGATAGCTTGGAAAAATCATCAAAATGGGTAACAGTTTCAGATTATTCAG
GAGAGGACCAAGAGTTTAGCCAGCCGTTAAACAACATTGGTGCCAAATATTGGAGAATAA
CAGTTGATACTAAGGGAGGACGTTACAATTGGCCATCACTTCCTGAGCTTCAAATCATTG
GTTATCAATTACCGGCTGCGGATCTTGTGATGGCAATGCTAGCTACTGCAGAGGAGCTAT
CTCAGCAAAAAGACAAGTTCTCTCAAGAGCAGCTTAAGGAGCTCGAAGTCAAAATAGCTG
CCTTAAAGGCTGCTTTAGATAGTAAGATGTTTAATGCCGATGCTATTAACGCTAGTACTG
CTGATCTGAAGGCTTATGTTGATAAGCTTTTAGCTGATAGAACTGATCAGGAAAAAGTAG
CTAAAGCAGCTAAAGTTGAGCAGCCTGTGGCTACTGACATAAAAGAAAATACTGAGCCAG
AAAATCCAAAGACAGACTAGCTTATCC.
Amino acid sequence of recombinant mature EndoSe
SEQ ID NO: 4
EDKVVQTSPSVSAIDDLHYL
SENSKKEFKEGLSKAGEVPEKLKDILSKAQQADKQAKVLAEMKVPEKIAMKPLKGPLYGG
YFRTWHDKTSDPAEKDKVNSMGELPKEVDLAFVFHDWTKDYSFFWQELATKHVPTLNKQG
TRVIRTIPWRFLAGGDHSGIAEDTQKYPNTPEGNKALAKAIVDEYVYKYNLDGLDVDIER
DSIPKVNGKESNENIQRSIAVFEEIGKLIGPKGADKSRLFIMDSTYMADKNPLIERGAQY
IDLLLVQVYGTQGEKGDWDPVARKPEKTMEERWESYSKYIRPEQYMVGFSFYEEYAGSGN
LWYDINERKDDHNPLNSEIAGTRAERYAKWQPKTGGVKGGIFSYAIDRDGVAHQPKKVSD
DEKRTNKAIKDITDGIVKSDYKVSKALKKVMENDKSYELIDQKDFPDKALREAVIAQVGS
RRGDLERFNGTLRLDNPDIKSLEGLNKLKKLAKLELIGLSQITKLDSLVLPANAKPTKDT
LANVLEAYDSAKKEETKAIPQVALTISGLTGLKELNLAGFDRDSLAGIDAASLTSLEKVD
LSSNKLDLAAGTENRQILDTMLATVTKHGGVSEKTFVFDHQKPTGLYPDTYGTKSLQLPV
ANDTIDLQAKLLFGTVTNQGTLINSEADYKAYQEQEIAGHRFVDSSYDYKAFAVTYKDYK
IKVTDSTLGVTDHKDLSTSKEETYKVEFFSPTNSTKPVHEAKVVVGAEKTMMVNLAEGAT
VIGGDADPTNAKKVFDGLLNNDTTILSTSNKASIIFELKEPGLVKYWRFFNDSKISKADC
IKEAKLEAFVGHLEAGSKVKDSLEKSSKWVTVSDYSGEDQEFSQPLNNIGAKYWRITVDT
KGGRYNWPSLPELQIIGYQLPAADLVMAMLATAEELSQQKDKFSQEQLKELEVKIAALKA
ALDSKMFNADAINASTADLKAYVDKLLADRTDQEKVAKAAKVEQPVATDIKENTEPENPK
TD
Nucleotide sequence of endoSe encoding the fragment A
SEQ ID NO: 5
GAGGATAAGGTTGTGCAAACTAGTCCATCAGTCTCTGCTATTGATGACC
TACATTACCTGTCGGAAAACAGTAAAAAAGAATTTAAGGAGGGGTTATCAAAGGCAGGAG
AAGTACCTGAAAAGCTAAAGGATATTTTATCCAAGGCACAGCAGGCAGATAAGCAGGCAA
AGGTTCTTGCAGAAATGAAGGTTCCTGAAAAAATAGCCATGAAGCCTTTAAAGGGGCCTC
TTTATGGTGGCTATTTTAGGACTTGGCATGATAAAACATCAGATCCGGCTGAAAAGGATA
AGGTTAATTCTATGGGAGAATTGCCTAAGGAGGTTGACTTAGCCTTTGTTTTCCATGATT
GGACCAAGGATTATAGCTTTTTCTGGCAAGAATTGGCGACCAAGCATGTGCCAACGCTGA
ACAAGCAGGGAACACGTGTGATTCGTACCATTCCATGGCGGTTCCTTGCAGGCGGTGATC
ATAGTGGTATTGCTGAAGATACGCAAAAATACCCAAATACTCCAGAGGGAAATAAGGCCT
TGGCAAAGGCTATTGTAGATGAATACGTTTATAAATATAATCTTGATGGTTTAGATGTTG
ATATTGAGCGGGATAGCATTCCAAAAGTAAATGGAAAAGAGAGTAACGAAAATATTCAGC
GCTCTATTGCTGTTTTTGAAGAAATTGGCAAGCTTATTGGGCCAAAGGGCGCTGACAAGT
CACGTTTGTTCATTATGGATAGCACCTACATGGCTGACAAGAACCCATTGATTGAGCGCG
GTGCCCAATAT
Amino acid sequence of the recombinant EndoSe fragment A
SEQ ID NO: 6
EDKVVQTSPSVSAIDDLHYL
SENSKKEFKEGLSKAGEVPEKLKDILSKAQQADKQAKVLAEMKVPEKIAMKPLKGPLYGG
YFRTWHDKTSDPAEKDKVNSMGELPKEVDLAFVFHDWTKDYSFFWQELATKHVPTLNKQG
TRVIRTIPWRFLAGGDHSGIAEDTQKYPNTPEGNKALAKAIVDEYVYKYNLDGLDVDIER
DSIPKVNGKESNENIQRSIAVFEEIGKLIGPKGADKSRLFIMDSTYMADKNPLIERGAQY
Nucleotide sequence of the endoSe encoding the fragment C
SEQ ID NO: 7
AAGGAGGCCAAGCTTGAAGCCTTTGTTGGCCATCTTGAAGCTGGCT
CAAAGGTAAAGGATAGCTTGGAAAAATCATCAAAATGGGTAACAGTTTCAGATTATTCAG
GAGAGGACCAAGAGTTTAGCCAGCCGTTAAACAACATTGGTGCCAAATATTGGAGAATAA
CAGTTGATACTAAGGGAGGACGTTACAATTGGCCATCACTTCCTGAGCTTCAAATCATTG
GTTATCAATTACCGGCTGCGGATCTTGTGATGGCAATGCTAGCTACTGCAGAGGAGCTAT
CTCAGCAAAAAGACAAGTTCTCTCAAGAGCAGCTTAAGGAGCTCGAAGTCAAAATAGCTG
CCTTAAAGGCTGCTTTAGATAGTAAGATGTTTAATGCCGATGCTATTAACGCTAGTACTG
CTGATCTGAAGGCTTATGTTGATAAGCTTTTAGCTGATAGAACTGATCAGGAAAAAGTAG
CTAAAGCAGCTAAAGTTGAGCAGCCTGTGGCTACTGACATAAAAGAAAATACTGAGCCAG
AAAATCCAAAGACAGACTAGCTTATCC
Amino acid sequence of the recombinant EndoSe fragment C
SEQ ID NO: 8
KEAKLEAFVGHLEAGSKVKDSLEKSSKWVTVSDYSGEDQEFSQPLNNIGAKYWRITVDT
KGGRYNWPSLPELQIIGYQLPAADLVMAMLATAEELSQQKDKFSQEQLKELEVKIAALKA
ALDSKMFNADAINASTADLKAYVDKLLADRTDQEKVAKAAKVEQPVATDIKENTEPENPK
TD
Amino acid sequence of recombinant fragment SEC2.16 of CNE
SEQ ID NO: 9
MATNLSDNITSLTVASSSLRDGERTTVKVAFDDKKQKIKAGDTIEVTWPTSGNVYIQGFNKTIPLNIR
GVDVGTLEVTLDKAVFTFNQNIETMHDVSGWGEFDITVRNVTQTTAETSGTTTVKVGNRTATITVTKP
EAGTGTSSFYYKTGDMQPNDTERVRWFLLINNNKEWVANTVTVEDDIQGGQTLDMSSFDITVSGYRNE
RFVGENALTEFHTTFPNSVITATDNHISVRLDQYDASQNTVNIAYKTKITDFDQKEFANNSKIWYQIL
YKDQVSGQESNHQVANINANGGVDGSRYTSFTVKKIWNDKENQDGKRPKTITVQLYANDQKVNDKTIE
LSDTNSWQASFGKLDKYDSQNQKITYSVKEVMVPVGYQSQVEGDSGVGFTITNTYTPEVISITGQKTW
DDRENQDGKRPKEITVRLLANDAATDKVATASEQTGWKYTFTNLPKYKDGKQITYTIQEDPVADYTTT
IQGFDITNHHEVALTSLKVIKVWNDKDDYYHKRPKEITILLKADGKVIREHQMTPDQQGKWEYTFDQL
PVYQTGKKISYSIEEKQVAGYQAPVYEVDEGLKQVTVTNTLNPSYKLEPG
Nucleotide sequence of the endoSz gene
SEQ ID NO: 10
ATGGA
AAAACAGGTGTTAGTCAAGAAAACACTGAAATGTGTTTGTGCTGCAGCGTTAATGGTGGC
TATATTAGCTGCCCAACATGATTCACTCGTAACAGTCAGGGCAGAGGATAAGGTTGTGCA
AACTAGTCCATCAGTCTCTGCTATTGATGACCTACATTATCTGTCGGAAAACAGTAAAAA
AGAATTTAAGGAGGAGTTATCAAAGGCAGGAGAAGTACCTGAAAAGCTAAAGGAGATTTT
ATCCAAGGCACAGCAGGCAGACAAGCAGGCAAAAACACTTGCTGAGATGAAGGTTCCCGA
AAAAATACCAATGAAGCCTTTAAAGGGGCCTCTTTATGGTGGTTATTTTAGGACCTGGCA
TGATAAAACATCAGACCCAGCTGAAAAGGATAAGGTTAATTCTATGGGAGAATTGCCCAA
GGAGGTTGACTTAGCCTTTGTTTTCCATGATTGGACCAAGGATTACAGCCTTTTTTGGCA
GAAATTGGCAACCAAGCATATCCCGAAATTAAACAAGCAGGGCACGCGCGTGATTCGTAC
GATTCCTTGGCGGTTTCTTGCAGGTGGTGACCATAGTGGTATTGCAGAAGATGCGCAAAA
ATACCCAAATACTCCAGAGGGAAATAAAGCTTTGGCCAAGGCTATTGTAGATGAATATGT
CTATAAATATAATCTCGATGGTTTAGATGTTGATATTGAGCGAGATAGCATTCCAAAAGT
AAATAAAGAGGAAAGCAAAGAGGGGATAGAACGCTCTATTCAGGTTTTTGAAGAAATTGG
CAAGCTTATTGGGCCAAAGGGGGCTGACAGGTCACGTTTGTTTATTATGGATAGCACCTA
CATGGCTGATAAGAACCCATTGATTGAGCGCGGTGCGCCATATATTGATTTGTTGCTTGT
GCAGGTTTATGGTGCTCAAGGTGAACGAGGAGAGTGGGACCCGGTTGCTAGAAAGCCTGA
AAAAACAATGGAGGAACGATGGGAGTCGTATAGCAAATACATTCGTCCTGAGCAGTACAT
GGTTGGCTTCTCCTTCTACGAGGAAAATGCGGGCAGTGGTAACCTCTGGTATGACATTAA
TGAAAGAAAAGATGATCATAATCCGTTACATTCAGAGATAACTGGTACTCGTGCTGAGCG
CTATGCAAAATGGCAGCCTAAGACAGGTGGTGTTAAGGGAGGGATTTTCTCTTATGCAAT
TGATCGTGACGGCGTAGCACATCAACCAGAAAAGTATGCCAAGCGTAAAGATTTTAAAGA
TGTAACAGATAAGATATTCCACTCTGATTACAAAGTATCAAAGGCCCTAAAAGAGGTCAT
GGTAAAGGACAAATCCTATGAGCAGATTGATGAAACAGATTTTCCAGACAAGGCTTTGCG
AGAAGCAGTTATTGCACAGGTTGGAAGCAGAAGAGGAGATTTGGAGCGCTTCAATGGCAC
CCTGCGCTTAGATAACCCAGATATCAAGAGCTTAGAAGGCCTAAATAACCTTAAAAAACT
AGCTAAGCTAGAGCTAGTTGGTTTATCGCAAATCACAAAGCTAGATCAATCAGTCCTGCC
TGAAAATATTAAGCCAACCAAGGATACGCTAGTCAGCGTTCTTGAAGCCTATAAAAAAGA
TGATCAGGAAGCAGCTAAGGCGATTCCACAGGTGGCTCTGACCATTTCTGGTCTAACTGG
CTTGAAGGAATTAAATCTTGCTGGCTTTGAGCGTGAGACCTTGGCTGGAATTGACGCAGC
TAGCCTAACCTCTCTTGAAAAGGTGGATCTCTCTAGCAATAAGCTGGACTTAGCAGCTGG
CACTGACAATCGTCAGATTCTTGATACCATGCTGGCAACAGTGACTAAGCATGGCAAAGC
CAATGCAGACAATATGACATTTGATCATCAAAAGCCTACCGGTCTTTATCCTGATACTTA
TGGCACTAAGAGCCTTCAGTTACCAGTAGCAAATGATACAATTGATTTGCAGGCTAAGCT
TTTATTTGGAACAGTTACTAATCAGGGCACGCTAATCAATAGCGAAGCTGACTATAAGGC
TTATCAGGAGCAGGAAATAGCAGGTCGCCGCTTTGTTGATCCAAGCTATGACTACAAAGC
TTTTGCAGTGACCTACGATGCTTATAAGGTCAGAGTGACTGACTCAACCTTAGGCGTTAC
TGATGAGAAGAAGCTCTCCACTAGCAAGGAGGAAACCTACAAGATTGAATTCTTTAGCCC
TACTAATAGCACTAAGCCTGTGCATGAGGCTAAGGTTGTCGTTGGTGAAGAAAAAACCAT
GATGGTCAACCTAGCAGAGGGAGCAACGATTATTGGGGGAAGTGCAGATCAAACAAATGC
AAAAAAAGTGTTTGATGGTTTGCTCAATAATGATACAACAACTCTGTCAACTAGCAATAA
AGCTTCTATCATTTTTGAACTTAAAGAGTCTGGCTTAGTCAAGCATTGGCGTTTCTTTAA
TGATAGTGCCAAAAAGAAAGAAGATTATATCAAGGAAGCTAAGCTTGAAGCCTTCGTTGG
TCATCTTGAGGACAGCTCAAAGGTGAAGGATAGCTTAGAAAAATCAACTGAATGGGTAAC
AGTTTCAGATTATTCAGGAGAGGCTCAAGAGTTTAGCCAGCCGTTAAACAACGTTGGTGC
CAAATATTGGAGAATAACAATTGACAATAAGAAAAGTCAATATGGATATGTCTCTCTTCC
TGAGCTGCAACTTATTGGTTATCAATTACCGGCTGCGTATCCTGTGATGGCAACGCTAGC
TGCTGCAGAGGAGCTATCTCAACAAAAAGACAAGTTCTCTCAAAAGCAGCTTAAGGAGCT
CGAAGTCAAAGTAGCTGCCTTAAAGGCTGCTTTAGATAATAAGATGTTTAATGCTGATAC
TATTAATGCTAGCTTTGCTGATGTGAAAGCTTATGTTGATAAGCTACTAGCAGACGCAGC
TGGCAAGAAAACACCAGGCAAAGCCACTAAGGAAGCTCAGCTAGTGACTACTGACGCAAA
AGAAAAGGCTGAGTCAGAAAAATCAAAGGCAGAC
Amino acid sequence of EndoSz
SEQ ID NO: 11
MEKQVLVKKTLKCVCAAALMVAILAAQHDSLVTVRAEDKVVQTSPSVSAIDDLHYLSENS
KKEFKEELSKAGEVPEKLKEILSKAQQADKQAKTLAEMKVPEKIPMKPLKGPLYGGYFRT
WHDKTSDPAEKDKVNSMGELPKEVDLAFVFHDWTKDYSLFWQKLATKHIPKLNKQGTRVI
RTIPWRFLAGGDHSGIAEDAQKYPNTPEGNKALAKAIVDEYVYKYNLDGLDVDIERDSIP
KVNKEESKEGIERSIQVFEEIGKLIGPKGADRSRLFIMDSTYMADKNPLIERGAPYIDLL
LVQVYGAQGERGEWDPVARKPEKTMEERWESYSKYIRPEQYMVGFSFYEENAGSGNLWYD
INERKDDHNPLHSEITGTRAERYAKWQPKTGGVKGGIFSYAIDRDGVAHQPEKYAKRKDF
KDVTDKIFHSDYKVSKALKEVMVKDKSYEQIDETDFPDKALREAVIAQVGSRRGDLERFN
GTLRLDNPDIKSLEGLNKLKKLAKLELVGLSQITKLDQSVLPENIKPTKDTLVSVLEAYK
KDDQEAAKAIPQVALTISGLTGLKELNLAGFERETLAGIDAASLTSLEKVDLSSNKLDLA
AGTDNRQILDTMLATVTKHGKANADNMTFDHQKPTGLYPDTYGTKSLQLPVANDTIDLQA
KLLFGTVTNQGTLINSEADYKAYQEQEIAGRRFVDPSYDYKAFAVTYDAYKVRVTDSTLG
VTDEKKLSTSKEETYKIEFFSPTNSTKPVHEAKVVVGEEKTMMVNLAEGATIIGGSADQT
NAKKVFDGLLNNDTTTLSTSNKASIIFELKESGLVKHWRFFNDSAKKKEDYIKEAKLEAF
VGHLEDSSKVKDSLEKSTEWVTVSDYSGEAQEFSQPLNNVGAKYWRITIDNKKSQYGYVS
LPELQLIGYQLPAAYPVMATLAAAEELSQQKDKFSQKQLKELEVKVAALKAALDNKMFNA
DTINASFADVKAYVDKLLADAAGKKTPGKATKEAQLVTTDAKEKAESEKSKAD
nucleotide sequence of the gene endoSz from subsp. zooepidemicus
encoding recombinant EndoSz lacking the signal sequence
SEQ ID NO: 12
GAGGATAAGGTTGTGCA
AACTAGTCCATCAGTCTCTGCTATTGATGACCTACATTATCTGTCGGAAAACAGTAAAAA
AGAATTTAAGGAGGAGTTATCAAAGGCAGGAGAAGTACCTGAAAAGCTAAAGGAGATTTT
ATCCAAGGCACAGCAGGCAGACAAGCAGGCAAAAACACTTGCTGAGATGAAGGTTCCCGA
AAAAATACCAATGAAGCCTTTAAAGGGGCCTCTTTATGGTGGTTATTTTAGGACCTGGCA
TGATAAAACATCAGACCCAGCTGAAAAGGATAAGGTTAATTCTATGGGAGAATTGCCCAA
GGAGGTTGACTTAGCCTTTGTTTTCCATGATTGGACCAAGGATTACAGCCTTTTTTGGCA
GAAATTGGCAACCAAGCATATCCCGAAATTAAACAAGCAGGGCACGCGCGTGATTCGTAC
GATTCCTTGGCGGTTTCTTGCAGGTGGTGACCATAGTGGTATTGCAGAAGATGCGCAAAA
ATACCCAAATACTCCAGAGGGAAATAAAGCTTTGGCCAAGGCTATTGTAGATGAATATGT
CTATAAATATAATCTCGATGGTTTAGATGTTGATATTGAGCGAGATAGCATTCCAAAAGT
AAATAAAGAGGAAAGCAAAGAGGGGATAGAACGCTCTATTCAGGTTTTTGAAGAAATTGG
CAAGCTTATTGGGCCAAAGGGGGCTGACAGGTCACGTTTGTTTATTATGGATAGCACCTA
CATGGCTGATAAGAACCCATTGATTGAGCGCGGTGCGCCATATATTGATTTGTTGCTTGT
GCAGGTTTATGGTGCTCAAGGTGAACGAGGAGAGTGGGACCCGGTTGCTAGAAAGCCTGA
AAAAACAATGGAGGAACGATGGGAGTCGTATAGCAAATACATTCGTCCTGAGCAGTACAT
GGTTGGCTTCTCCTTCTACGAGGAAAATGCGGGCAGTGGTAACCTCTGGTATGACATTAA
TGAAAGAAAAGATGATCATAATCCGTTACATTCAGAGATAACTGGTACTCGTGCTGAGCG
CTATGCAAAATGGCAGCCTAAGACAGGTGGTGTTAAGGGAGGGATTTTCTCTTATGCAAT
TGATCGTGACGGCGTAGCACATCAACCAGAAAAGTATGCCAAGCGTAAAGATTTTAAAGA
TGTAACAGATAAGATATTCCACTCTGATTACAAAGTATCAAAGGCCCTAAAAGAGGTCAT
GGTAAAGGACAAATCCTATGAGCAGATTGATGAAACAGATTTTCCAGACAAGGCTTTGCG
AGAAGCAGTTATTGCACAGGTTGGAAGCAGAAGAGGAGATTTGGAGCGCTTCAATGGCAC
CCTGCGCTTAGATAACCCAGATATCAAGAGCTTAGAAGGCCTAAATAAGCTTAAAAAACT
AGCTAAGCTAGAGCTAGTTGGTTTATCGCAAATCACAAAGCTAGATCAATCAGTCCTGCC
TGAAAATATTAAGCCAACCAAGGATACGCTAGTCAGCGTTCTTGAAGCCTATAAAAAAGA
TGATCAGGAAGCAGCTAAGGCGATTCCACAGGTGGCTCTGACCATTTCTGGTCTAACTGG
CTTGAAGGAATTAAATCTTGCTGGCTTTGAGCGTGAGACCTTGGCTGGAATTGACGCAGC
TAGCCTAACCTCTCTTGAAAAGGTGGATCTCTCTAGCAATAAGCTGGACTTAGCAGCTGG
CACTGACAATCGTCAGATTCTTGATACCATGCTGGCAACAGTGACTAAGCATGGCAAAGC
CAATGCAGACAATATGACATTTGATCATCAAAAGCCTACCGGTCTTTATCCTGATACTTA
TGGCACTAAGAGCCTTCAGTTACCAGTAGCAAATGATACAATTGATTTGCAGGCTAAGCT
TTTATTTGGAACAGTTACTAATCAGGGCACGCTAATCAATAGCGAAGCTGACTATAAGGC
TTATCAGGAGCAGGAAATAGCAGGTCGCCGCTTTGTTGATCCAAGCTATGACTACAAAGC
TTTTGCAGTGACCTACGATGCTTATAAGGTCAGAGTGACTGACTCAACCTTAGGCGTTAC
TGATGAGAAGAAGCTCTCCACTAGCAAGGAGGAAACCTACAAGATTGAATTCTTTAGCCC
TACTAATAGCACTAAGCCTGTGCATGAGGCTAAGGTTGTCGTTGGTGAAGAAAAAACCAT
GATGGTCAACCTAGCAGAGGGAGCAACGATTATTGGGGGAAGTGCAGATCAAACAAATGC
AAAAAAAGTGTTTGATGGTTTGCTCAATAATGATACAACAACTCTGTCAACTAGCAATAA
AGCTTCTATCATTTTTGAACTTAAAGAGTCTGGCTTAGTCAAGCATTGGCGTTTCTTTAA
TGATAGTGCCAAAAAGAAAGAAGATTATATCAAGGAAGCTAAGCTTGAAGCCTTCGTTGG
TCATCTTGAGGACAGCTCAAAGGTGAAGGATAGCTTAGAAAAATCAACTGAATGGGTAAC
AGTTTCAGATTATTCAGGAGAGGCTCAAGAGTTTAGCCAGCCGTTAAACAACGTTGGTGC
CAAATATTGGAGAATAACAATTGACAATAAGAAAAGTCAATATGGATATGTCTCTCTTCC
TGAGCTGCAACTTATTGGTTATCAATTACCGGCTGCGTATCCTGTGATGGCAACGCTAGC
TGCTGCAGAGGAGCTATCTCAACAAAAAGACAAGTTCTCTCAAAAGCAGCTTAAGGAGCT
CGAAGTCAAAGTAGCTGCCTTAAAGGCTGCTTTAGATAATAAGATGTTTAATGCTGATAC
TATTAATGCTAGCTTTGCTGATGTGAAAGCTTATGTTGATAAGCTACTAGCAGACGCAGC
TGGCAAGAAAACACCAGGCAAAGCCACTAAGGAAGCTCAGCTAGTGACTACTGACGCAAA
AGAAAAGGCTGAGTCAGAAAAATCAAAGGCAGAC
amino acid sequence of recombinant EndoSz encoded by SEQ ID 12
SEQ ID NO: 13
EDKVVQTSPSVSAIDDLHYLSENS
KKEFKEELSKAGEVPEKLKEILSKAQQADKQAKTLAEMKVPEKIPMKPLKGPLYGGYFRT
WHDKTSDPAEKDKVNSMGELPKEVDLAFVFHDWTKDYSLFWQKLATKHIPKLNKQGTRVI
RTIPWRFLAGGDHSGIAEDAQKYPNTPEGNKALAKAIVDEYVYKYNLDGLDVDIERDSIP
KVNKEESKEGIERSIQVFEEIGKLIGPKGADRSRLFIMDSTYMADKNPLIERGAPYIDLL
LVQVYGAQGERGEWDPVARKPEKTMEERWESYSKYIRPEQYMVGFSFYEENAGSGNLWYD
INERKDDHNPLHSEITGTRAERYAKWQPKTGGVKGGIFSYAIDRDGVAHQPEKYAKRKDF
KDVTDKIFHSDYKVSKALKEVMVKDKSYEQIDETDFPDKALREAVIAQVGSRRGDLERFN
GTLRLDNPDIKSLEGLNKLKKLAKLELVGLSQITKLDQSVLPENIKPTKDTLVSVLEAYK
KDDQEAAKAIPQVALTISGLTGLKELNLAGFERETLAGIDAASLTSLEKVDLSSNKLDLA
AGTDNRQILDTMLATVTKHGKANADNMTFDHQKPTGLYPDTYGTKSLQLPVANDTIDLQA
KLLFGTVTNQGTLINSEADYKAYQEQEIAGRRFVDPSYDYKAFAVTYDAYKVRVTDSTLG
VTDEKKLSTSKEETYKIEFFSPTNSTKPVHEAKVVVGEEKTMMVNLAEGATIIGGSADQT
NAKKVFDGLLNNDTTTLSTSNKASIIFELKESGLVKHWRFFNDSAKKKEDYIKEAKLEAF
VGHLEDSSKVKDSLEKSTEWVTVSDYSGEAQEFSQPLNNVGAKYWRITIDNKKSQYGYVS
LPELQLIGYQLPAAYPVMATLAAAFELSQQKDKESQKQLKELEVKVAALKAALDNKMENA
DTINASFADVKAYVDKLLADAAGKKTPGKATKEAQLVTTDAKEKAESEKSKAD
Truncated nucleotide sequence of GenBank: AF296340.1
>gi|12656366|gb|AF296340.1|AF296340 Streptococcus pyogenes secreted
endoglycosidase EndoS (ndoS) gene, complete cds
SEQ ID NO: 14
ATG
GATAAACATTTGTTGGTAAAAAGAACACTAGGGTGTGTTTGTGCTGCAACGTTGATGGGAGCTGCCTTAG
CGACCCACCATGATTCACTCAATACTGTAAAAGCGGAGGAGAAGACTGTTCAGGTTCAGAAAGGATTACC
TTCTATCGATAGCTTGCATTATCTGTCAGAGAATAGCAAAAAAGAATTTAAAGAAGAACTCTCAAAAGCG
GGGCAAGAATCTCAAAAGGTCAAAGAGATATTAGCAAAAGCTCAGCAGGCAGATAAACAAGCTCAAGAAC
TTGCCAAAATGAAAATTCCTGAGAAAATACCGATGAAACCGTTACATGGTCCTCTCTACGGTGGTTACTT
TAGAACTTGGCATGACAAAACATCAGATCCAACAGAAAAAGACAAAGTTAACTCGATGGGAGAGCTTCCT
AAAGAAGTAGATCTAGCCTTTATTTTCCACGATTGGACAAAAGATTATAGCCTTTTTTGGAAAGAATTGG
CCACCAAACATGTGCCAAAGTTAAACAAGCAAGGGACACGTGTCATTCGTACCATTCCATGGCGTTTCCT
AGCTGGGGGTGATAACAGTGGTATTGCAGAAGATACCAGTAAATACCCAAATACACCAGAGGGAAATAAA
GCTTTAGCCAAAGCTATTGTTGATGAATATGTTTATAAATACAACCTTGATGGCTTAGATGTGGATGTTG
AACATGATAGTATTCCAAAAGTTGACAAAAAAGAAGATACAGCAGGCGTAGAACGCTCTATTCAAGTGTT
TGAAGAAATTGGGAAATTAATTGGACCAAAAGGTGTTGATAAATCGCGGTTATTTATTATGGATAGCACC
TACATGGCTGATAAAAACCCATTGATTGAGCGAGGAGCTCCTTATATTAATTTATTACTGGTACAGGTCT
ATGGTTCACAAGGAGAGAAAGGTGGTTGGGAGCCTGTTTCTAATCGACCTGAAAAAACAATGGAAGAACG
ATGGCAAGGTTATAGCAAGTATATTCGTCCTGAACAATACATGATTGGTTTTTCTTTCTATGAGGAAAAT
GCTCAAGAAGGGAATCTTTGGTATGATATTAATTCTCGCAAGGACGAGGACAAAGCAAATGGAATTAACA
CTGACATAACTGGAACGCGTGCCGAACGGTATGCAAGGTGGCAACCTAAGACAGGTGGGGTTAAGGGAGG
TATCTTCTCCTACGCTATTGACCGAGATGGTGTAGCTCATCAACCTAAAAAATATGCTAAACAGAAAGAG
TTTAAGGACGCAACTGATAACATCTTCCACTCAGATTATAGTGTCTCCAAGGCATTAAAGACAGTTATGC
TAAAAGATAAGTCGTATGATCTGATTGATGAGAAAGATTTCCCAGATAAGGCTTTGCGAGAAGCTGTGAT
GGCGCAGGTTGGAACCAGAAAAGGTGATTTGGAACGTTTCAATGGCACATTACGATTGGATAATCCAGCG
ATTCAAAGTTTAGAAGGTCTAAATAAATTTAAAAAATTAGCTCAATTAGACTTGATTGGCTTATCTCGCA
TTACAAAGCTCGACCGTTCTGTTTTACCCGCTAATATGAAGCCAGGCAAAGATACCTTGGAAACAGTTCT
TGAAACCTATAAAAAGGATAACAAAGAAGAACCTGCTACTATCCCACCAGTATCTTTGAAGGTTTCTGGT
TTAACTGGTCTGAAAGAATTAGATTTGTCAGGTTTTGACCGTGAAACCTTGGCTGGTCTTGATGCCGCTA
CTCTAACGTCTTTAGAAAAAGTTGATATTTCTGGCAACAAACTTGATTTGGCTCCAGGAACAGAAAATCG
ACAAATTTTTGATACTATGCTATCAACTATCAGCAATCATGTTGGAAGCAATGAACAAACAGTGAAATTT
GACAAGCAAAAACCAACTGGGCATTACCCAGATACCTATGGGAAAACTAGTCTGCGCTTACCAGTGGCAA
ATGAAAAAGTTGATTTGCAAAGCCAGCTTTTGTTTGGGACTGTGACAAATCAAGGAACCCTAATCAATAG
CGAAGCAGACTATAAGGCTTACCAAAATCATAAAATTGCTGGACGTAGCTTTGTTGATTCAAACTATCAT
TACAATAACTTTAAAGTTTCTTATGAGAACTATACCGTTAAAGTAACTGATTCCACATTGGGAACCACTA
CTGACAAAACGCTAGCAACTGATAAAGAAGAGACCTATAAGGTTGACTTCTTTAGCCCAGCAGATAAGAC
AAAAGCTGTTCATACTGCTAAAGTGATTGTTGGTGACGAAAAAACCATGATGGTTAATTTGGCAGAAGGC
GCAACAGTTATTGGAGGAAGTGCTGATCCTGTAAATGCAAGAAAGGTATTTGATGGGCAACTGGGCAGTG
AGACTGATAATATCTCTTTAGGATGGGATTCTAAGCAAAGTATTATATTTAAATTGAAAGAAGATGGATT
AATAAAGCATTGGCGTTTCTTCAATGATTCAGCCCGAAATCCTGAGACAACCAATAAACCTATTCAGGAA
GCAAGTCTACAAATTTTTAATATCAAAGATTATAATCTAGATAATTTGTTGGAAAATCCCAATAAATTTG
ATGATGAAAAATATTGGATTACTGTAGATACTTACAGTGCACAAGGAGAGAGAGCTACTGCATTCAGTAA
TACATTAAATAATATTACTAGTAAATATTGGCGAGTTGTCTTTGATACTAAAGGAGATAGATATAGTTCG
CCAGTAGTCCCTGAACTCCAAATTTTAGGTTATCCGTTACCTAACGCCGACACTATCATGAAAACAGTAA
CTACTGCTAAAGAGTTATCTCAACAAAAAGATAAGTTTTCTCAAAAGATGCTTGATGAGTTAAAAATAAA
AGAGATGGCTTTAGAAACTTCTTTGAACAGTAAGATTTTTGATGTAACTGCTATTAATGCTAATGCTGGA
GTTTTGAAAGATTGTATTGAGAAAAGGCAGCTGCTAAAAAAA
Amino acid sequence of EndoS from S. pyogenes GenBank: AAK00850.1
SEQ ID NO: 15
MDKHLLVKRTLGCVCAATLMGAALATHHDSLNTVKAEEKTVQVQKGLPSIDSLHYLSENSKKEFKEELSK
AGQESQKVKEILAKAQQADKQAQELAKMKIPEKIPMKPLHGPLYGGYFRTWHDKTSDPTEKDKVNSMGEL
PKEVDLAFIFHDWTKDYSLFWKELATKHVPKLNKQGTRVIRTIPWRFLAGGDNSGIAEDTSKYPNTPEGN
KALAKAIVDEYVYKYNLDGLDVDVEHDSIPKVDKKEDTAGVERSIQVFEEIGKLIGPKGVDKSRLFIMDS
TYMADKNPLIERGAPYINLLLVQVYGSQGEKGGWEPVSNRPEKTMEERWQGYSKYIRPEQYMIGFSFYEE
NAQEGNLWYDINSRKDEDKANGINTDITGTRAERYARWQPKTGGVKGGIFSYAIDRDGVAHQPKKYAKQK
EFKDATDNIFHSDYSVSKALKTVMLKDKSYDLIDEKDFPDKALREAVMAQVGTRKGDLERFNGTLRLDNP
AIQSLEGLNKFKKLAQLDLIGLSRITKLDRSVLPANMKPGKDTLETVLETYKKDNKEEPATIPPVSLKVS
GLTGLKELDLSGFDRETLAGLDAATLTSLEKVDISGNKLDLAPGTENRQIFDTMLSTISNHVGSNEQTVK
FDKQKPTGHYPDTYGKTSLRLPVANEKVDLQSQLLFGTVTNQGTLINSEADYKAYQNHKIAGRSFVDSNY
HYNNFKVSYENYTVKVTDSTLGTTTDKTLATDKEETYKVDFFSPADKTKAVHTAKVIVGDEKTMMVNLAE
GATVIGGSADPVNARKVFDGQLGSETDNISLGWDSKQSIIFKLKEDGLIKHWRFFNDSARNPETTNKPIQ
EASLQIFNIKDYNLDNLLENPNKFDDEKYWITVDTYSAQGERATAFSNTLNNITSKYWRVVFDTKGDRYS
SPVVPELQILGYPLPNADTIMKTVTTAKELSQQKDKFSQKMLDELKIKEMALETSLNSKIFDVTAINANA
GVLKDCIEKRQLLKK
Nucleotide sequence of the ndoS gene (SEQ ID NO: 14) lacking the sequence
encoding the signal sequence
SEQ ID NO: 16
GAGGAGAAGACTGTTCAGGTTCAGAAAGGATTACC
TTCTATCGATAGCTTGCATTATCTGTCAGAGAATAGCAAAAAAGAATTTAAAGAAGAACTCTCAAAAGCG
GGGCAAGAATCTCAAAAGGTCAAAGAGATATTAGCAAAAGCTCAGCAGGCAGATAAACAAGCTCAAGAAC
TTGCCAAAATGAAAATTCCTGAGAAAATACCGATGAAACCGTTACATGGTCCTCTCTACGGTGGTTACTT
TAGAACTTGGCATGACAAAACATCAGATCCAACAGAAAAAGACAAAGTTAACTCGATGGGAGAGCTTCCT
AAAGAAGTAGATCTAGCCTTTATTTTCCACGATTGGACAAAAGATTATAGCCTTTTTTGGAAAGAATTGG
CCACCAAACATGTGCCAAAGTTAAACAAGCAAGGGACACGTGTCATTCGTACCATTCCATGGCGTTTCCT
AGCTGGGGGTGATAACAGTGGTATTGCAGAAGATACCAGTAAATACCCAAATACACCAGAGGGAAATAAA
GCTTTAGCCAAAGCTATTGTTGATGAATATGTTTATAAATACAACCTTGATGGCTTAGATGTGGATGTTG
AACATGATAGTATTCCAAAAGTTGACAAAAAAGAAGATACAGCAGGCGTAGAACGCTCTATTCAAGTGTT
TGAAGAAATTGGGAAATTAATTGGACCAAAAGGTGTTGATAAATCGCGGTTATTTATTATGGATAGCACC
TACATGGCTGATAAAAACCCATTGATTGAGCGAGGAGCTCCTTATATTAATTTATTACTGGTACAGGTCT
ATGGTTCACAAGGAGAGAAAGGTGGTTGGGAGCCTGTTTCTAATCGACCTGAAAAAACAATGGAAGAACG
ATGGCAAGGTTATAGCAAGTATATTCGTCCTGAACAATACATGATTGGTTTTTCTTTCTATGAGGAAAAT
GCTCAAGAAGGGAATCTTTGGTATGATATTAATTCTCGCAAGGACGAGGACAAAGCAAATGGAATTAACA
CTGACATAACTGGAACGCGTGCCGAACGGTATGCAAGGTGGCAACCTAAGACAGGTGGGGTTAAGGGAGG
TATCTTCTCCTACGCTATTGACCGAGATGGTGTAGCTCATCAACCTAAAAAATATGCTAAACAGAAAGAG
TTTAAGGACGCAACTGATAACATCTTCCACTCAGATTATAGTGTCTCCAAGGCATTAAAGACAGTTATGC
TAAAAGATAAGTCGTATGATCTGATTGATGAGAAAGATTTCCCAGATAAGGCTTTGCGAGAAGCTGTGAT
GGCGCAGGTTGGAACCAGAAAAGGTGATTTGGAACGTTTCAATGGCACATTACGATTGGATAATCCAGCG
ATTCAAAGTTTAGAAGGTCTAAATAAATTTAAAAAATTAGCTCAATTAGACTTGATTGGCTTATCTCGCA
TTACAAAGCTCGACCGTTCTGTTTTACCCGCTAATATGAAGCCAGGCAAAGATACCTTGGAAACAGTTCT
TGAAACCTATAAAAAGGATAACAAAGAAGAACCTGCTACTATCCCACCAGTATCTTTGAAGGTTTCTGGT
TTAACTGGTCTGAAAGAATTAGATTTGTCAGGTTTTGACCGTGAAACCTTGGCTGGTCTTGATGCCGCTA
CTCTAACGTCTTTAGAAAAAGTTGATATTTCTGGCAACAAACTTGATTTGGCTCCAGGAACAGAAAATCG
ACAAATTTTTGATACTATGCTATCAACTATCAGCAATCATGTTGGAAGCAATGAACAAACAGTGAAATTT
GACAAGCAAAAACCAACTGGGCATTACCCAGATACCTATGGGAAAACTAGTCTGCGCTTACCAGTGGCAA
ATGAAAAAGTTGATTTGCAAAGCCAGCTTTTGTTTGGGACTGTGACAAATCAAGGAACCCTAATCAATAG
CGAAGCAGACTATAAGGCTTACCAAAATCATAAAATTGCTGGACGTAGCTTTGTTGATTCAAACTATCAT
TACAATAACTTTAAAGTTTCTTATGAGAACTATACCGTTAAAGTAACTGATTCCACATTGGGAACCACTA
CTGACAAAACGCTAGCAACTGATAAAGAAGAGACCTATAAGGTTGACTTCTTTAGCCCAGCAGATAAGAC
AAAAGCTGTTCATACTGCTAAAGTGATTGTTGGTGACGAAAAAACCATGATGGTTAATTTGGCAGAAGGC
GCAACAGTTATTGGAGGAAGTGCTGATCCTGTAAATGCAAGAAAGGTATTTGATGGGCAACTGGGCAGTG
AGACTGATAATATCTCTTTAGGATGGGATTCTAAGCAAAGTATTATATTTAAATTGAAAGAAGATGGATT
AATAAAGCATTGGCGTTTCTTCAATGATTCAGCCCGAAATCCTGAGACAACCAATAAACCTATTCAGGAA
GCAAGTCTACAAATTTTTAATATCAAAGATTATAATCTAGATAATTTGTTGGAAAATCCCAATAAATTTG
ATGATGAAAAATATTGGATTACTGTAGATACTTACAGTGCACAAGGAGAGAGAGCTACTGCATTCAGTAA
TACATTAAATAATATTACTAGTAAATATTGGCGAGTTGTCTTTGATACTAAAGGAGATAGATATAGTTCG
CCAGTAGTCCCTGAACTCCAAATTTTAGGTTATCCGTTACCTAACGCCGACACTATCATGAAAACAGTAA
CTACTGCTAAAGAGTTATCTCAACAAAAAGATAAGTTTTCTCAAAAGATGCTTGATGAGTTAAAAATAAA
AGAGATGGCTTTAGAAACTTCTTTGAACAGTAAGATTTTTGATGTAACTGCTATTAATGCTAATGCTGGA
GTTTTGAAAGATTGTATTGAGAAAAGGCAGCTGCTAAAAAAA
Amino acid sequence of EndoS from S. pyogenes (SEQ ID NO: 16 lacking the
signal sequence)
SEQ ID NO: 17
EEKTVQVQKGLPSIDSLHYLSENSKKEFKEELSK
AGQESQKVKEILAKAQQADKQAQELAKMKIPEKIPMKPLHGPLYGGYFRTWHDKTSDPTEKDKVNSMGEL
PKEVDLAFIFHDWTKDYSLFWKELATKHVPKLNKQGTRVIRTIPWRFLAGGDNSGIAEDTSKYPNTPEGN
KALAKAIVDEYVYKYNLDGLDVDVEHDSIPKVDKKEDTAGVERSIQVFEEIGKLIGPKGVDKSRLFIMDS
TYMADKNPLIERGAPYINLLLVQVYGSQGEKGGWEPVSNRPEKTMEERWQGYSKYIRPEQYMIGFSFYEE
NAQEGNLWYDINSRKDEDKANGINTDITGTRAERYARWQPKTGGVKGGIFSYAIDRDGVAHQPKKYAKQK
EFKDATDNIFHSDYSVSKALKTVMLKDKSYDLIDEKDFPDKALREAVMAQVGTRKGDLERFNGTLRLDNP
AIQSLEGLNKFKKLAQLDLIGLSRITKLDRSVLPANMKPGKDTLETVLETYKKDNKEEPATIPPVSLKVS
GLTGLKELDLSGFDRETLAGLDAATLTSLEKVDISGNKLDLAPGTENRQIFDTMLSTISNHVGSNEQTVK
FDKQKPTGHYPDTYGKTSLRLPVANEKVDLQSQLLFGTVTNQGTLINSEADYKAYQNHKIAGRSFVDSNY
HYNNFKVSYENYTVKVTDSTLGTTTDKTLATDKEETYKVDFFSPADKTKAVHTAKVIVGDEKTMMVNLAE
GATVIGGSADPVNARKVFDGQLGSETDNISLGWDSKQSIIFKLKEDGLIKHWRFFNDSARNPETTNKPIQ
EASLQIFNIKDYNLDNLLENPNKFDDEKYWITVDTYSAQGERATAFSNTLNNITSKYWRVVFDTKGDRYS
SPVVPELQILGYPLPNADTIMKTVTTAKELSQQKDKFSQKMLDELKIKEMALETSLNSKIFDVTAINANA
GVLKDCIEKRQLLKK
Nucleotide sequence of primer Eq61p1
SEQ ID NO: 18
GTCGGATCCGAGGATAAGGTTGTGCAAACTAG
Nucleotide sequence of primer Eq61p6
SEQ ID NO: 19
GCCTCTCGAGGGATAAGCTAGTCTGTCTTTGG
Nucleotide sequence of primer Eq61p2
SEQ ID NO: 20
GCAGCTCGAGTTAATATTGGGCACCGCGCTCAATC
Nucleotide sequence of primer Eq61p5
SEQ ID NO: 21
TGACGGATCCAAGGAGGCCAAGCTTGAAGC
Nucleotide sequence of primer Eq61p3
SEQ ID NO: 22
TTATGGATCCGATCGCGATGGTGTAGCG
Nucleotide sequence of primer Eq61p4
SEQ ID NO: 23
TCTTCTCGAGTTAACCGCCATGCTTAGTCACTG
Nucleotide sequence of primer GexS
SEQ ID NO: 24
GGGCTGGCAAGCCACGTTTGGTG
Nucleotide sequence of primer GexR
SEQ ID NO: 25
CCGGGAGCTGCATGTGTCAGAGG

### References

1.) Albert, H., Collin, M., Dudziak, D., Ravetch, J. and Nimmerjahn, F. (2008). PNAS 105: 15005-15009.
2.) Allhorn M, and Collin M. Ann N Y Acad Sci. 2009 September;1173:664-9
3.) Allhorn, M, Olin, A. I. Nimmerjahn, F. and Collin, M. PLoS ONE (www.plosone.orq) January 2008. Issue 1. e1413. Open access.
4.) Allhorn, M., Olsen, A and Collin, M. BMC Microbiology 2008 8:3 (http://www.biomedcentral.com/1471-2180/8/3) Open access.
5.) Barnham, M., A. Ljunggren, and M. McIntyre. 1987. Human infection with Streptococcus zooepidemicus (Lancefield group C): three case reports. Epidem. Inf. 98: 183-190.
6.) Bisno A L, Brito M O, Collins CM. (2003) Lancet Infect Dis. April; 3(4):191-200. Review.
7.) Chhatwal G S, McMillan D J. (2005) Trends Mol Med. April; 11(4):152-5. Review
8.) Collin, M. and Olsen, A. (2001). EMBO J 20:3046-3055.
9.) Collin M, Olsen A. (2003) Infect Immun. June; 71(6):2983-92. Review
10.) Fernandez, E. et al. 2004. Int. J. Syst. Evol. Microbiol. 54: 2291-2296.
11.) Flock, M., Jacobsson, K., Frykberg, L., Hirst, T., R., Franklin, A., Guss, B. and Flock, J.-I. (2004) Infect Immun 72:3228-3236.
12.) Flock M, Karlstrom A, Lannergard J, Guss B, Flock J.-I. (2006) Vaccine. May 8; 24(19):4144-51.
13.) Guss, B., Flock, M., Frykberg, L., Waller, A., Robinson, C., Smith,K. and Flock, J.-I.: Available from Nature Precedings <http://hdl.handle.net/10101/npre.2009.2985.1>(2009) Posted 26 Mar. 2009.
14.) Guss B, Flock M, Frykberg L, Waller AS, Robinson C, et al. (2009) Getting to Grips with Strangles: An Effective Multi-Component Recombinant Vaccine for the Protection of Horses from Streptococcus equi Infection. PLoS Pathog 5(9): e1000584. doi:10.1371/journal.ppat.1000. Sep. 18, 2009.
15.) Holden M T, Heather Z, Paillot R, Steward K F, Webb K, et al. (2009) Genomic evidence for the evolution of Streptococcus equi: host restriction, increased virulence, and genetic exchange with human pathogens. PLoS Pathog 5: e1000346.
16.) Hulting, G. et al 2009 FEMS Microbiol Lett. 298:44-50.
17.) Jacobs, A. A, Goovaerts, D., Nuijten, P. J., Theelen, R. P., Hartford, O. M., et al. (2000) Investigations towards an efficacious and safe strangles vaccine: submucosal vaccination with a live attenuated Streptococcus equi. Vet Rec 147: 563-567
18.) Jacobsson, K., Jonsson, H., Lindmark, H., Guss, B., Lindberg, M., and Frykberg. L. (1997) Shot-gun phage display mapping of two streptococcal cell-surface proteins. Microbiol Res. 152:1-8.
19.) Janulczyk, R. and Rasmussen, M. (2001) Infect Immun 4019-4026
20.) Jonsson, H., Lindmark, H., and Guss. B. (1995) A protein G related cell surface protein in Streptococcus zooepidemicus. Infect Immun 63:2968-2975.
21.) Karlström, A. et al (2004) Vet Microbiol. Dec 9;104(3-4):179-88.
22.) Karlström, A. et al (2006) Vet Microbiol. Apr 16;114(1-2):72-81.
23.) Kemp-Symonds J, Kemble T, Waller A (2007) Modified live Streptococcus equi (‘strangles’) vaccination followed by clinically adverse reactions associated with bacterial replication. Equine Vet J 39: 284-286.
24.) Lannerg\dot{a}rd, J. (2006) Potentially virulence-related extracellular proteins of Streptococcus equi. (Doctoral thesis) Acta Universitatis Agriculturae Sueciae, Agraria 2006:80. ISBN 91-576-7129-X
25.) Lannerg\dot{a}rd, J., Frykberg, L. and Guss B. (2003) CNE, a collagen-binding protein of Streptrococcus equi. FEMS Microbiol. Lett. 222:69-74.
26.) Lannergard, J. and Guss, B. (2006) FEMS Microbiol Lett 262: 230-235.
27.) Lindmark, H. (1999) Characterization of adhesive extracellular proteins from Streptococcus equi. (Doctoral thesis) Acta Universitatis Agriculturae Sueciae, Agraria 139. ISBN 91-576-5488-3
28.) Lindmark, H., and Guss, B. (1999) SFS, a novel fibronectin-binding protein from Streptococcus equi, inhibits the binding between fibronectin and collagen. Infect. Immun. 67: 2383-2388.
29.) Lindmark, H., Jacobsson, K., Frykberg, L., and Guss, B. (1996) Fibronectin-binding protein of Streptococcus equi subspecies zooepidemicus. Infect Immun 64:3993-3999.
30.) Lindmark, H., Jonsson, P., Olsson-Engvall, E., and Guss, B. (1999) Pulsed-field gel electrophoresis and distribution of the genes zag and fnz in isolates of Streptococcus equi. Res Vet Sci. 66:93-99.
31.) Lindmark, H., Nilsson, M., and Guss, B. (2001) Comparison of the fibronectin-binding protein FNE from Streptococcus equi subspecies equi with FNZ from S. equi subspecies zooepidemicus reveals a major and conserved difference. Infect immun 69: 3159-3163.
32.) Morein, B. and Lovgren Bengtsson. K. (1998) Immunology and Cellbiology 76:295-299.
33.) Nakata, M. et al (2009) Infect Immun 77:32-44.
34.) Nandakumar, K.S., Collin, M. Olsen, M. et al. 2007. Eur.J. Immunol. 37:2973-2982.
35.) Newton R, Waller A, King, A (2005) Investigation of suspected adverse reactions following strangles vaccination in horses. Vet Rec 156: 291-292.
36.) Rasmussen, M. et al (1999) J Biol Chem 274: 15336-15344.
37.) Schneewind, O., Fowler, A. and Faull, K. F. (1995) Structure of the cell wall anchor of surface proteins in Staphylococcus aureus. Science 268:103-106.
38.) Sutcliffe I C, Harrington D J. (2002) Microbiology. July; 148(Pt 7):2065-77.
39.) Sweeney et al (2005) J Vet Int Med 19:123-134.
40.) Timoney J F. (2004) Vet Res. 35:397-409
41.) Timoney J F, Kumar P (2008) Early pathogenesis of equine Streptococcus equi in fection (strangles). Equine Vet J 40: 637-642.
42.) Timoney J F, Qin A, Muthupalani S, Artiushin S (2007) Vaccine potential of novel surface exposed and secreted proteins of Streptococcus equi. Vaccine 25: 5583-5590.
43.) Turner C E, et al. (2009) Vaccine. August 6; 27(36):4923-9. Epub 2009 Jun. 27.
44.) Walker, J. A. and Timoney, J. F. (2002) Vet Microbiol 89:311-321.
45.) Waller, A., Flock, M., Smith, K., Robinson, C., Mitchell, Z., Karlstrom, A., Lannerg\dot{a}rd, J., Bergman, R., Guss, B. and Flock, J.-I. (2007) Vaccine 25: 3629-3635.

1. An antigenic composition comprising at least one antigen, wherein said at least one antigen comprises at least part of a protein selected from EndoSe of Streptococcus equi subsp. equi and EndoSz of Streptococcus equi subsp. zooepidemicus, and wherein said at least part of said protein comprises at least one antigenic epitope.

2. The antigenic composition of claim 1, wherein the at least one antigen is selected from EndoSe comprising the amino acid sequence of SEQ ID NO: 2; EndoSe comprising the amino acid sequence of SEQ ID NO: 4; fragment A of EndoSe comprising the amino acid sequence of SEQ ID NO: 6; fragment C of EndoSe comprising the amino acid sequence of SEQ ID NO: 8; EndoSz comprising the amino acid sequence of SEQ ID NO: 11; and EndoSz comprising the amino acid sequence of SEQ ID NO: 13.

3. An antigenic composition comprising at least one antigen, wherein said at least one antigen comprises at least part of a protein selected from EndoSe of Streptococcus equi subsp. equi, EndoSz of Streptococcus equi subsp. zooepidemicus or EndoS of Streptococcus pyogenes, and wherein said at least part of said protein comprises at least one antigenic epitope; or an analog thereof; and at least one further antigen selected from extracellular proteins, or fragments thereof, expressed extracellularly in pathogenic streptococci.

4. The antigenic composition of claim 3, wherein the extracellular proteins, or fragments thereof, comprise CNE, FNZ, SclC, SFS, EAG, IdeE, IdeE2, Eq5, Eq8, IdeZ2, Eqz5, Eqz8 and antigenic fragments thereof.

5. The antigenic composition of claim 3, wherein the at least one antigen is selected from EndoSe comprising the amino acid sequence of SEQ ID NO: 2; EndoSe comprising the amino acid sequence of SEQ ID NO: 4; fragment A of EndoSe comprising the amino acid sequence of SEQ ID NO 6; fragment C of EndoSe comprising the amino acid sequence of SEQ ID NO: 8; EndoSz comprising the amino acid sequence of SEQ ID NO: 11; EndoSz comprising the amino acid sequence of SEQ ID NO: 13; EndoS comprising the amino acid sequence of SEQ ID NO: 15; and EndoS comprising the amino acid sequence of SEQ ID NO: 17.

6. The antigenic composition of claim 1, wherein the antigenic composition is immunogenic.

7. The antigenic composition of claim 1, wherein at least one antigen is an isolated or purified antigen.

8. The antigenic composition of claim 1, wherein at least one antigen is recombinantly produced.

9. A vaccine composition for protecting a mammal against infection with Streptococcus equi subsp. equi and/or subsp. zooepidemicus or Streptococcus pyogenes, which comprises as immunizing component an antigenic composition comprising at least one antigen which comprises at least part of a protein selected from EndoSe of Streptococcus equi subsp. equi, EndoSz of Streptococcus equi subsp. zooepidemicus or EndoS of Streptococcus pyogenes, and wherein said at least part of said protein comprises at least one antigenic epitope; or an analog thereof; and a pharmaceutically acceptable carrier.

10. The vaccine composition of claim 9, which comprises as immunizing component an antigenic composition of comprising at least one antigen, wherein said at least one antigen comprises at least part of a protein selected from EndoSe of Streptococcus equi subsp. equi and EndoSz of Streptococcus equi subsp. zooepidemicus, and wherein said at least part of said protein comprises at least one antigenic epitope.

11. The vaccine composition of claim 9, which further comprises an adjuvant.

12. A vaccine composition for protecting a mammal against infection with Streptococcus equi subsp. equi and/or subsp. zooepidemicus or Streptococcus pyogenes, which comprises at least one recombinant vector and at least one polynucleotide inserted therein that encodes at least one antigen which comprises at least part of a protein selected from EndoSe of Streptococcus equi subsp. equi, EndoSz of Streptococcus equi subsp. zooepidemicus or EndoS of Streptococcus pyogenes, and wherein said at least part of said protein comprises at least one antigenic epitope, or an analog thereof, and which vector is capable of expressing said at least one antigen in vivo in a mammal susceptible to infection with Streptococcus equi subsp. equi, and/or subsp. zooepidemicus or Streptococcus pyogenes.

13. The vaccine composition of claim 12, wherein the vector is an expression vector in the form of a plasmid or a viral vector.

14. The vaccine composition of claim 12, wherein the at least one polynucleotide is selected from polynucleotides comprising the sequences of SEQ ID NO: 1, SEQ ID NO: 3, SEQ ID NO: 5, SEQ ID NO: 7, SEQ ID NO: 10, SEQ ID NO: 12, SEQ ID NO: 14, or SEQ ID NO: 16.

15. The vaccine composition of claim 9, which is provided in a physiologically administrable form, preferably a form that is administrable by intramuscular, intradermal, subcutaneous or intranasal inoculation.

16. The vaccine composition of claim 9, wherein the vaccine is capable of protecting susceptible mammals against infection with Streptococcus equi or Streptococcus pyogenes, especially horses against strangles caused by Streptococcus equi subsp. equi.

17. The vaccine composition of claim 16, which is capable of stimulating serum, mucosal and/or bronchial antibody responses directed against Streptococcus equi or pyogenes antigens in mammals susceptible to Streptococcus equi or pyogenes, especially horses.

18. A method for producing an antigen of an antigenic composition of claim 1, which method comprises
(a) providing a DNA fragment encoding said antigen and introducing said fragment into an expression vector;
(b) introducing said vector, which contains said DNA fragment, into a compatible host cell;
(c) culturing said host cell provided in step (b) under conditions required for expression of the product encoded by said DNA fragment; and
(d) isolating the expressed product from the cultured host cell, and, optionally,
(e) purifying the isolated product from step (d) by a chromatographic method.

19. A method for the preparation of a vaccine composition according to claim 1, which vaccine composition comprises as immunizing component an antigenic composition comprising at least one antigen which comprises at least part of a protein selected from EndoSe of Streptococcus equi subsp. equi, EndoSz of Streptococcus equi subsp. zooepidemicus and EndoS of Streptococcus pyogenes, and wherein said at least part of said protein comprises at least one antigenic epitope; or an analog thereof, especially the antigenic composition of any one of claim 1, wherein the method comprises mixing the antigenic composition with a pharmaceutically acceptable carrier.

20. (canceled)

21. (canceled)

22. A method for the production of an antiserum, which method comprises administering an antigenic composition comprising at least one antigen which comprises at least part of a protein selected from EndoSe of Streptococcus equi subsp. equi, EndoSz of Streptococcus equi subsp. zooepidemicus and EndoS of Streptococcus pyogenes, and wherein said at least part of said protein comprises at least one antigenic epitope; or an analog thereof, especially the antigenic composition of claim 1, to a mammal (preferably non-human) host to produce antibodies in said host and recovering antiserum containing said antibodies produced in said host.

23. A method of prophylactic or therapeutic treatment of Streptococcus equi or Streptococcus pyogenes infection in a mammal, especially a horse, comprising administering to said mammal an immunologically effective amount of a vaccine composition of claim 9.

24. A method of protecting horses against Streptococcus equi infection, which comprises inoculating a horse subcutaneously, intradermally, intramuscularly or intranasally with a vaccine composition of claim 9 to induce an immune response against Streptococcus equi in said horse.

25. The method of claim 24, wherein an immune response in the form of IgG and/or IgA and/or IgM antibodies in serum and/or the nasopharyngeal mucus is induced in said horse.

26. An antibody preparation comprising at least one, and preferably at least two monoclonal or polyclonal antibodies, or antibody fragments, specific for an antigen of an antigenic composition comprising at least one antigen which comprises at least part of a protein selected from EndoSe of Streptococcus equi subsp. equi, EndoSz of Streptococcus equi subsp. zooepidemicus and EndoS of Streptococcus pyogenes, and wherein said at least part of said protein comprises at least one antigenic epitope; or an analog thereof, especially the antigenic composition of claim 1.

27. The antibody preparation of claim 26 which is to be used prophylactically or therapeutically to provide passive immunization when administered to a mammal susceptible to infection by Streptococcus equi or Streptococcus pyogenes, or infected by Streptococcus equi or Streptococcus pyogenes.

28. A method of passive immunization which comprises administering to a mammal an antibody preparation of claim 26.

29. Use of an antigen selected from EndoSe of Streptococcus equi subsp. equi, EndoSz of Streptococcus equi subsp. zooepidemicus and EndoS of Streptococcus pyogenes, antigenic fragments thereof, analogs thereof and antigenic analog fragments, as enhancer in a vaccine composition, especially a vaccine composition against infection by Streptococcus equi or Streptococcus pyogenes.

30. Use of an antigen selected from EndoSe of Streptococcus equi subsp. equi, EndoSz of Streptococcus equi subsp. zooepidemicus and EndoS of Streptococcus pyogenes, antigenic fragments thereof, analogs thereof and antigenic analog fragments, as a diagnostic tool to determine antibody titers against the antigens in sera from mammals.

31. Use of an antigen selected from EndoSe of Streptococcus equi subsp. equi, EndoSz of Streptococcus equi subsp. zooepidemicus and EndoS of Streptococcus pyogenes, antigenic fragments thereof, analogs thereof and antigenic analog fragments, as a diagnostic tool to determine the enzymatically inhibitory activity of antibodies in sera from mammals.


SEQUENCE LISTING (26 sequences)

SEQ ID NO: 1 - DNA, 3054 bp, Streptococcus equi
atggaaaaacaggtgttagtcaagaaaacactgaaatgtgtttgtgctgcagcgttaatggtggctatattagctgcccaacatgattcactcataagggtcaaggcagaggataaggttgtgcaaactagtccatcagtctctgctattgatgacctacattacctgtcggaaaacagtaaaaaagaatttaaggaggggttatcaaaggcaggagaagtacctgaaaagctaaaggatattttatccaaggcacagcaggcagataagcaggcaaaggttcttgcagaaatgaaggttcctgaaaaaatagccatgaagcctttaaaggggcctctttatggtggctattttaggacttggcatgataaaacatcagatccggctgaaaaggataaggttaattctatgggagaattgcctaaggaggttgacttagcctttgttttccatgattggaccaaggattatagctttttctggcaagaattggcgaccaagcatgtgccaacgctgaacaagcagggaacacgtgtgattcgtaccattccatggcggttccttgcaggcggtgatcatagtggtattgctgaagatacgcaaaaatacccaaatactccagagggaaataaggccttggcaaaggctattgtagatgaatacgtttataaatataatcttgatggtttagatgttgatattgagcgggatagcattccaaaagtaaatggaaaagagagtaacgaaaatattcagcgctctattgctgtttttgaagaaattggcaagcttattgggccaaagggcgctgacaagtcacgtttgttcattatggatagcacctacatggctgacaagaacccattgattgagcgcggtgcccaatatattgatttgctgcttgtgcaggtttatggcactcaaggtgagaagggagattgggatccagtcgctagaaaacctgaaaagacaatggaggaacgttgggaatcgtatagcaaatacattcgtcctgagcagtacatggttggtttttctttctatgaggaaaatgcgggcagtggtaacctctggtatgatattaatgagaggaaagatgatcataatccgttaaattcagagatagctggtactcgtgctgagcgttatgcaaaatggcagcctaagacaggtggtgtcaagggagggattttctcttatgcgattgatcgcgatggtgtagcgcatcaacctaaaaaagtctcagatgatgagaaaagaactaacaaggctataaaggatataacagatggtattgtcaaatcagattataaggtttctaaggccttgaagaaggttatggaaaatgacaaatcctatgagctgattgatcagaaagattttccagacaaggctttgcgagaagcagttattgcacaggttggaagcagaagaggggatttagagcggttcaatggaaccctgcgcttagacaatccggatatcaagagtttagaaggcctgaataagcttaaaaaactagctaagctagagctaatcggtctatcacaaatcacaaagctggatagcttagtcctacctgcaaatgctaagccgaccaaggatacgctggccaatgttcttgaagcctacgacagcgctaagaaggaagagactaaggcgattccacaggtggctctgaccatttctggtctaactggcttgaaggaattaaatcttgctggctttgatcgtgatagcttggctggaattgacgcagctagcctaacctctcttgaaaaggtggatctctctagtaataagctggacttagcagctggtacggaaaatcgtcagattcttgataccatgctggcaacagtgactaagcatggcggtgttagcgaaaagacgtttgtatttgatcatcaaaagcctactggtctttatcctgatacttatggcactaagagccttcagttaccagtagcaaatgatacaattgatttgcaggctaagcttttatttggaacagttaccaatcagggcacgctaatcaatagcgaagctgactataaggcttatcaggagcaggaaatagcaggtcaccgttttgttgattcaagctatgattacaaagcctttgcagtgacctacaaggactataagatcaaggtgactgactcaaccttaggtgtcactgatcacaaggacttatccactagcaaggaggagacctacaaggttgaattctttagccctactaatagcactaagcctgtgcatgaggctaaggttgtcgttggtgcggaaaaaaccatgatggttaacctagcagagggagcaactgtgattggtggtgatgcagatccaacaaatgcaaaaaaagtgtttgatggtttgctcaataatgatacaacaattctgtcaactagcaataaagcttctatcatttttgaacttaaagagcctggcttagtcaagtattggcgtttctttaatgacagcaaaattagtaaagctgactgtattaaggaggccaagcttgaagcctttgttggccatcttgaagctggctcaaaggtaaaggatagcttggaaaaatcatcaaaatgggtaacagtttcagattattcaggagaggaccaagagtttagccagccgttaaacaacattggtgccaaatattggagaataacagttgatactaagggaggacgttacaattggccatcacttcctgagcttcaaatcattggttatcaattaccggctgcggatcttgtgatggcaatgctagctactgcagaggagctatctcagcaaaaagacaagttctctcaagagcagcttaaggagctcgaagtcaaaatagctgccttaaaggctgctttagatagtaagatgtttaatgccgatgctattaacgctagtactgctgatctgaaggcttatgttgataagcttttagctgatagaactgatcaggaaaaagtagctaaagcagctaaagttgagcagcctgtggctactgacataaaagaaaatactgagccagaaaatccaaagacagac

SEQ ID NO: 2 - protein, 1018 aa, Streptococcus equi
MEKQVLVKKTLKCVCAAALMVAILAAQHDSLIRVKAEDKVVQTSPSVSAIDDLHYLSENSKKEFKEGLSKAGEVPEKLKDILSKAQQADKQAKVLAEMKVPEKIAMKPLKGPLYGGYFRTWHDKTSDPAEKDKVNSMGELPKEVDLAFVFHDWTKDYSFFWQELATKHVPTLNKQGTRVIRTIPWRFLAGGDHSGIAEDTQKYPNTPEGNKALAKAIVDEYVYKYNLDGLDVDIERDSIPKVNGKESNENIQRSIAVFEEIGKLIGPKGADKSRLFIMDSTYMADKNPLIERGAQYIDLLLVQVYGTQGEKGDWDPVARKPEKTMEERWESYSKYIRPEQYMVGFSFYEENAGSGNLWYDINERKDDHNPLNSEIAGTRAERYAKWQPKTGGVKGGIFSYAIDRDGVAHQPKKVSDDEKRTNKAIKDITDGIVKSDYKVSKALKKVMENDKSYELIDQKDFPDKALREAVIAQVGSRRGDLERFNGTLRLDNPDIKSLEGLNKLKKLAKLELIGLSQITKLDSLVLPANAKPTKDTLANVLEAYDSAKKEETKAIPQVALTISGLTGLKELNLAGFDRDSLAGIDAASLTSLEKVDLSSNKLDLAAGTENRQILDTMLATVTKHGGVSEKTFVFDHQKPTGLYPDTYGTKSLQLPVANDTIDLQAKLLFGTVTNQGTLINSEADYKAYQEQEIAGHRFVDSSYDYKAFAVTYKDYKIKVTDSTLGVTDHKDLSTSKEETYKVEFFSPTNSTKPVHEAKVVVGAEKTMMVNLAEGATVIGGDADPTNAKKVFDGLLNNDTTILSTSNKASIIFELKEPGLVKYWRFFNDSKISKADCIKEAKLEAFVGHLEAGSKVKDSLEKSSKWVTVSDYSGEDQEFSQPLNNIGAKYWRITVDTKGGRYNWPSLPELQIIGYQLPAADLVMAMLATAEELSQQKDKFSQEQLKELEVKIAALKAALDSKMFNADAINASTADLKAYVDKLLADRTDQEKVAKAAKVEQPVATDIKENTEPENPKTD

SEQ ID NO: 3 - DNA, 2956 bp, Streptococcus equi
gaggataaggttgtgcaaactagtccatcagtctctgctattgatgacctacattacctgtcggaaaacagtaaaaaagaatttaaggaggggttatcaaaggcaggagaagtacctgaaaagctaaaggatattttatccaaggcacagcaggcagataagcaggcaaaggttcttgcagaaatgaaggttcctgaaaaaatagccatgaagcctttaaaggggcctctttatggtggctattttaggacttggcatgataaaacatcagatccggctgaaaaggataaggttaattctatgggagaattgcctaaggaggttgacttagcctttgttttccatgattggaccaaggattatagctttttctggcaagaattggcgaccaagcatgtgccaacgctgaacaagcagggaacacgtgtgattcgtaccattccatggcggttccttgcaggcggtgatcatagtggtattgctgaagatacgcaaaaatacccaaatactccagagggaaataaggccttggcaaaggctattgtagatgaatacgtttataaatataatcttgatggtttagatgttgatattgagcgggatagcattccaaaagtaaatggaaaagagagtaacgaaaatattcagcgctctattgctgtttttgaagaaattggcaagcttattgggccaaagggcgctgacaagtcacgtttgttcattatggatagcacctacatggctgacaagaacccattgattgagcgcggtgcccaatatattgatttgctgcttgtgcaggtttatggcactcaaggtgagaagggagattgggatccagtcgctagaaaacctgaaaagacaatggaggaacgttgggaatcgtatagcaaatacattcgtcctgagcagtacatggttggtttttctttctatgaggaatatgcgggcagtggtaacctctggtatgatattaatgagaggaaagatgatcataatccgttaaattcagagatagctggtactcgtgctgagcgttatgcaaaatggcagcctaagacaggtggtgtcaagggagggattttctcttatgcgattgatcgcgatggtgtagcgcatcaacctaaaaaagtctcagatgatgagaaaagaactaacaaggctataaaggatataacagatggtattgtcaaatcagattataaggtttctaaggccttgaagaaggttatggaaaatgacaaatcctatgagctgattgatcagaaagattttccagacaaggctttgcgagaagcagttattgcacaggttggaagcagaagaggggatttagagcggttcaatggaaccctgcgcttagacaatccggatatcaagagtttagaaggcctgaataagcttaaaaaactagctaagctagagctaatcggtctatcacaaatcacaaagctggatagcttagtcctacctgcaaatgctaagccgaccaaggatacgctggccaatgttcttgaagcctacgacagcgctaagaaggaagagactaaggcgattccacaggtggctctgaccatttctggtctaactggcttgaaggaattaaatcttgctggctttgatcgtgatagcttggctggaattgacgcagctagcctaacctctcttgaaaaggtggatctctctagtaataagctggacttagcagctggtacggaaaatcgtcagattcttgataccatgctggcaacagtgactaagcatggcggtgttagcgaaaagacgtttgtatttgatcatcaaaagcctactggtctttatcctgatacttatggcactaagagccttcagttaccagtagcaaatgatacaattgatttgcaggctaagcttttatttggaacagttaccaatcagggcacgctaatcaatagcgaagctgactataaggcttatcaggagcaggaaatagcaggtcaccgttttgttgattcaagctatgattacaaagcctttgcagtgacctacaaggactataagatcaaggtgactgactcaaccttaggtgtcactgatcacaaggacttatccactagcaaggaggagacctacaaggttgaattctttagccctactaatagcactaagcctgtgcatgaggctaaggttgtcgttggtgcggaaaaaaccatgatggttaacctagcagagggagcaactgtgattggtggtgatgcagatccaacaaatgcaaaaaaagtgtttgatggtttgctcaataatgatacaacaattctgtcaactagcaataaagcttctatcatttttgaacttaaagagcctggcttagtcaagtattggcgtttctttaatgacagcaaaattagtaaagctgactgtattaaggaggccaagcttgaagcctttgttggccatcttgaagctggctcaaaggtaaaggatagcttggaaaaatcatcaaaatgggtaacagtttcagattattcaggagaggaccaagagtttagccagccgttaaacaacattggtgccaaatattggagaataacagttgatactaagggaggacgttacaattggccatcacttcctgagcttcaaatcattggttatcaattaccggctgcggatcttgtgatggcaatgctagctactgcagaggagctatctcagcaaaaagacaagttctctcaagagcagcttaaggagctcgaagtcaaaatagctgccttaaaggctgctttagatagtaagatgtttaatgccgatgctattaacgctagtactgctgatctgaaggcttatgttgataagcttttagctgatagaactgatcaggaaaaagtagctaaagcagctaaagttgagcagcctgtggctactgacataaaagaaaatactgagccagaaaatccaaagacagactagcttatcc

SEQ ID NO: 4 - protein, 982 aa, Streptococcus equi
EDKVVQTSPSVSAIDDLHYLSENSKKEFKEGLSKAGEVPEKLKDILSKAQQADKQAKVLAEMKVPEKIAMKPLKGPLYGGYFRTWHDKTSDPAEKDKVNSMGELPKEVDLAFVFHDWTKDYSFFWQELATKHVPTLNKQGTRVIRTIPWRFLAGGDHSGIAEDTQKYPNTPEGNKALAKAIVDEYVYKYNLDGLDVDIERDSIPKVNGKESNENIQRSIAVFEEIGKLIGPKGADKSRLFIMDSTYMADKNPLIERGAQYIDLLLVQVYGTQGEKGDWDPVARKPEKTMEERWESYSKYIRPEQYMVGFSFYEEYAGSGNLWYDINERKDDHNPLNSEIAGTRAERYAKWQPKTGGVKGGIFSYAIDRDGVAHQPKKVSDDEKRTNKAIKDITDGIVKSDYKVSKALKKVMENDKSYELIDQKDFPDKALREAVIAQVGSRRGDLERFNGTLRLDNPDIKSLEGLNKLKKLAKLELIGLSQITKLDSLVLPANAKPTKDTLANVLEAYDSAKKEETKAIPQVALTISGLTGLKELNLAGFDRDSLAGIDAASLTSLEKVDLSSNKLDLAAGTENRQILDTMLATVTKHGGVSEKTFVFDHQKPTGLYPDTYGTKSLQLPVANDTIDLQAKLLFGTVTNQGTLINSEADYKAYQEQEIAGHRFVDSSYDYKAFAVTYKDYKIKVTDSTLGVTDHKDLSTSKEETYKVEFFSPTNSTKPVHEAKVVVGAEKTMMVNLAEGATVIGGDADPTNAKKVFDGLLNNDTTILSTSNKASIIFELKEPGLVKYWRFFNDSKISKADCIKEAKLEAFVGHLEAGSKVKDSLEKSSKWVTVSDYSGEDQEFSQPLNNIGAKYWRITVDTKGGRYNWPSLPELQIIGYQLPAADLVMAMLATAEELSQQKDKFSQEQLKELEVKIAALKAALDSKMFNADAINASTADLKAYVDKLLADRTDQEKVAKAAKVEQPVATDIKENTEPENPKTD

SEQ ID NO: 5 - DNA, 780 bp, Streptococcus equi
gaggataaggttgtgcaaactagtccatcagtctctgctattgatgacctacattacctgtcggaaaacagtaaaaaagaatttaaggaggggttatcaaaggcaggagaagtacctgaaaagctaaaggatattttatccaaggcacagcaggcagataagcaggcaaaggttcttgcagaaatgaaggttcctgaaaaaatagccatgaagcctttaaaggggcctctttatggtggctattttaggacttggcatgataaaacatcagatccggctgaaaaggataaggttaattctatgggagaattgcctaaggaggttgacttagcctttgttttccatgattggaccaaggattatagctttttctggcaagaattggcgaccaagcatgtgccaacgctgaacaagcagggaacacgtgtgattcgtaccattccatggcggttccttgcaggcggtgatcatagtggtattgctgaagatacgcaaaaatacccaaatactccagagggaaataaggccttggcaaaggctattgtagatgaatacgtttataaatataatcttgatggtttagatgttgatattgagcgggatagcattccaaaagtaaatggaaaagagagtaacgaaaatattcagcgctctattgctgtttttgaagaaattggcaagcttattgggccaaagggcgctgacaagtcacgtttgttcattatggatagcacctacatggctgacaagaacccattgattgagcgcggtgcccaatat

SEQ ID NO: 6 - protein, 260 aa, Streptococcus equi
EDKVVQTSPSVSAIDDLHYLSENSKKEFKEGLSKAGEVPEKLKDILSKAQQADKQAKVLAEMKVPEKIAMKPLKGPLYGGYFRTWHDKTSDPAEKDKVNSMGELPKEVDLAFVFHDWTKDYSFFWQELATKHVPTLNKQGTRVIRTIPWRFLAGGDHSGIAEDTQKYPNTPEGNKALAKAIVDEYVYKYNLDGLDVDIERDSIPKVNGKESNENIQRSIAVFEEIGKLIGPKGADKSRLFIMDSTYMADKNPLIERGAQY

SEQ ID NO: 7 - DNA, 553 bp, Streptococcus equi
aaggaggccaagcttgaagcctttgttggccatcttgaagctggctcaaaggtaaaggatagcttggaaaaatcatcaaaatgggtaacagtttcagattattcaggagaggaccaagagtttagccagccgttaaacaacattggtgccaaatattggagaataacagttgatactaagggaggacgttacaattggccatcacttcctgagcttcaaatcattggttatcaattaccggctgcggatcttgtgatggcaatgctagctactgcagaggagctatctcagcaaaaagacaagttctctcaagagcagcttaaggagctcgaagtcaaaatagctgccttaaaggctgctttagatagtaagatgtttaatgccgatgctattaacgctagtactgctgatctgaaggcttatgttgataagcttttagctgatagaactgatcaggaaaaagtagctaaagcagctaaagttgagcagcctgtggctactgacataaaagaaaatactgagccagaaaatccaaagacagactagcttatcc

SEQ ID NO: 8 - protein, 181 aa, Streptococcus equi
KEAKLEAFVGHLEAGSKVKDSLEKSSKWVTVSDYSGEDQEFSQPLNNIGAKYWRITVDTKGGRYNWPSLPELQIIGYQLPAADLVMAMLATAEELSQQKDKFSQEQLKELEVKIAALKAALDSKMFNADAINASTADLKAYVDKLLADRTDQEKVAKAAKVEQPVATDIKENTEPENPKTD

SEQ ID NO: 9 - protein, 594 aa, Streptococcus equi
MATNLSDNITSLTVASSSLRDGERTTVKVAFDDKKQKIKAGDTIEVTWPTSGNVYIQGFNKTIPLNIRGVDVGTLEVTLDKAVFTFNQNIETMHDVSGWGEFDITVRNVTQTTAETSGTTTVKVGNRTATITVTKPEAGTGTSSFYYKTGDMQPNDTERVRWFLLINNNKEWVANTVTVEDDIQGGQTLDMSSFDITVSGYRNERFVGENALTEFHTTFPNSVITATDNHISVRLDQYDASQNTVNIAYKTKITDFDQKEFANNSKIWYQILYKDQVSGQESNHQVANINANGGVDGSRYTSFTVKKIWNDKENQDGKRPKTITVQLYANDQKVNDKTIELSDTNSWQASFGKLDKYDSQNQKITYSVKEVMVPVGYQSQVEGDSGVGFTITNTYTPEVISITGQKTWDDRENQDGKRPKEITVRLLANDAATDKVATASEQTGWKYTFTNLPKYKDGKQITYTIQEDPVADYTTTIQGFDITNHHEVALTSLKVIKVWNDKDDYYHKRPKEITILLKADGKVIREHQMTPDQQGKWEYTFDQLPVYQTGKKISYSIEEKQVAGYQAPVYEVDEGLKQVTVTNTLNPSYKLEPG

SEQ ID NO: 10 - DNA, 3039 bp, Streptococcus equi
atggaaaaacaggtgttagtcaagaaaacactgaaatgtgtttgtgctgcagcgttaatggtggctatattagctgcccaacatgattcactcgtaacagtcagggcagaggataaggttgtgcaaactagtccatcagtctctgctattgatgacctacattatctgtcggaaaacagtaaaaaagaatttaaggaggagttatcaaaggcaggagaagtacctgaaaagctaaaggagattttatccaaggcacagcaggcagacaagcaggcaaaaacacttgctgagatgaaggttcccgaaaaaataccaatgaagcctttaaaggggcctctttatggtggttattttaggacctggcatgataaaacatcagacccagctgaaaaggataaggttaattctatgggagaattgcccaaggaggttgacttagcctttgttttccatgattggaccaaggattacagccttttttggcagaaattggcaaccaagcatatcccgaaattaaacaagcagggcacgcgcgtgattcgtacgattccttggcggtttcttgcaggtggtgaccatagtggtattgcagaagatgcgcaaaaatacccaaatactccagagggaaataaagctttggccaaggctattgtagatgaatatgtctataaatataatctcgatggtttagatgttgatattgagcgagatagcattccaaaagtaaataaagaggaaagcaaagaggggatagaacgctctattcaggtttttgaagaaattggcaagcttattgggccaaagggggctgacaggtcacgtttgtttattatggatagcacctacatggctgataagaacccattgattgagcgcggtgcgccatatattgatttgttgcttgtgcaggtttatggtgctcaaggtgaacgaggagagtgggacccggttgctagaaagcctgaaaaaacaatggaggaacgatgggagtcgtatagcaaatacattcgtcctgagcagtacatggttggcttctccttctacgaggaaaatgcgggcagtggtaacctctggtatgacattaatgaaagaaaagatgatcataatccgttacattcagagataactggtactcgtgctgagcgctatgcaaaatggcagcctaagacaggtggtgttaagggagggattttctcttatgcaattgatcgtgacggcgtagcacatcaaccagaaaagtatgccaagcgtaaagattttaaagatgtaacagataagatattccactctgattacaaagtatcaaaggccctaaaagaggtcatggtaaaggacaaatcctatgagcagattgatgaaacagattttccagacaaggctttgcgagaagcagttattgcacaggttggaagcagaagaggagatttggagcgcttcaatggcaccctgcgcttagataacccagatatcaagagcttagaaggcctaaataagcttaaaaaactagctaagctagagctagttggtttatcgcaaatcacaaagctagatcaatcagtcctgcctgaaaatattaagccaaccaaggatacgctagtcagcgttcttgaagcctataaaaaagatgatcaggaagcagctaaggcgattccacaggtggctctgaccatttctggtctaactggcttgaaggaattaaatcttgctggctttgagcgtgagaccttggctggaattgacgcagctagcctaacctctcttgaaaaggtggatctctctagcaataagctggacttagcagctggcactgacaatcgtcagattcttgataccatgctggcaacagtgactaagcatggcaaagccaatgcagacaatatgacatttgatcatcaaaagcctaccggtctttatcctgatacttatggcactaagagccttcagttaccagtagcaaatgatacaattgatttgcaggctaagcttttatttggaacagttactaatcagggcacgctaatcaatagcgaagctgactataaggcttatcaggagcaggaaatagcaggtcgccgctttgttgatccaagctatgactacaaagcttttgcagtgacctacgatgcttataaggtcagagtgactgactcaaccttaggcgttactgatgagaagaagctctccactagcaaggaggaaacctacaagattgaattctttagccctactaatagcactaagcctgtgcatgaggctaaggttgtcgttggtgaagaaaaaaccatgatggtcaacctagcagagggagcaacgattattgggggaagtgcagatcaaacaaatgcaaaaaaagtgtttgatggtttgctcaataatgatacaacaactctgtcaactagcaataaagcttctatcatttttgaacttaaagagtctggcttagtcaagcattggcgtttctttaatgatagtgccaaaaagaaagaagattatatcaaggaagctaagcttgaagccttcgttggtcatcttgaggacagctcaaaggtgaaggatagcttagaaaaatcaactgaatgggtaacagtttcagattattcaggagaggctcaagagtttagccagccgttaaacaacgttggtgccaaatattggagaataacaattgacaataagaaaagtcaatatggatatgtctctcttcctgagctgcaacttattggttatcaattaccggctgcgtatcctgtgatggcaacgctagctgctgcagaggagctatctcaacaaaaagacaagttctctcaaaagcagcttaaggagctcgaagtcaaagtagctgccttaaaggctgctttagataataagatgtttaatgctgatactattaatgctagctttgctgatgtgaaagcttatgttgataagctactagcagacgcagctggcaagaaaacaccaggcaaagccactaaggaagctcagctagtgactactgacgcaaaagaaaaggctgagtcagaaaaatcaaaggcagac

SEQ ID NO: 11 - protein, 1013 aa, Streptococcus equi
MEKQVLVKKTLKCVCAAALMVAILAAQHDSLVTVRAEDKVVQTSPSVSAIDDLHYLSENSKKEFKEELSKAGEVPEKLKEILSKAQQADKQAKTLAEMKVPEKIPMKPLKGPLYGGYFRTWHDKTSDPAEKDKVNSMGELPKEVDLAFVFHDWTKDYSLFWQKLATKHIPKLNKQGTRVIRTIPWRFLAGGDHSGIAEDAQKYPNTPEGNKALAKAIVDEYVYKYNLDGLDVDIERDSIPKVNKEESKEGIERSIQVFEEIGKLIGPKGADRSRLFIMDSTYMADKNPLIERGAPYIDLLLVQVYGAQGERGEWDPVARKPEKTMEERWESYSKYIRPEQYMVGFSFYEENAGSGNLWYDINERKDDHNPLHSEITGTRAERYAKWQPKTGGVKGGIFSYAIDRDGVAHQPEKYAKRKDFKDVTDKIFHSDYKVSKALKEVMVKDKSYEQIDETDFPDKALREAVIAQVGSRRGDLERFNGTLRLDNPDIKSLEGLNKLKKLAKLELVGLSQITKLDQSVLPENIKPTKDTLVSVLEAYKKDDQEAAKAIPQVALTISGLTGLKELNLAGFERETLAGIDAASLTSLEKVDLSSNKLDLAAGTDNRQILDTMLATVTKHGKANADNMTFDHQKPTGLYPDTYGTKSLQLPVANDTIDLQAKLLFGTVTNQGTLINSEADYKAYQEQEIAGRRFVDPSYDYKAFAVTYDAYKVRVTDSTLGVTDEKKLSTSKEETYKIEFFSPTNSTKPVHEAKVVVGEEKTMMVNLAEGATIIGGSADQTNAKKVFDGLLNNDTTTLSTSNKASIIFELKESGLVKHWRFFNDSAKKKEDYIKEAKLEAFVGHLEDSSKVKDSLEKSTEWVTVSDYSGEAQEFSQPLNNVGAKYWRITIDNKKSQYGYVSLPELQLIGYQLPAAYPVMATLAAAEELSQQKDKFSQKQLKELEVKVAALKAALDNKMFNADTINASFADVKAYVDKLLADAAGKKTPGKATKEAQLVTTDAKEKAESEKSKAD

SEQ ID NO: 12 - DNA, 2931 bp, Streptococcus equi
gaggataaggttgtgcaaactagtccatcagtctctgctattgatgacctacattatctgtcggaaaacagtaaaaaagaatttaaggaggagttatcaaaggcaggagaagtacctgaaaagctaaaggagattttatccaaggcacagcaggcagacaagcaggcaaaaacacttgctgagatgaaggttcccgaaaaaataccaatgaagcctttaaaggggcctctttatggtggttattttaggacctggcatgataaaacatcagacccagctgaaaaggataaggttaattctatgggagaattgcccaaggaggttgacttagcctttgttttccatgattggaccaaggattacagccttttttggcagaaattggcaaccaagcatatcccgaaattaaacaagcagggcacgcgcgtgattcgtacgattccttggcggtttcttgcaggtggtgaccatagtggtattgcagaagatgcgcaaaaatacccaaatactccagagggaaataaagctttggccaaggctattgtagatgaatatgtctataaatataatctcgatggtttagatgttgatattgagcgagatagcattccaaaagtaaataaagaggaaagcaaagaggggatagaacgctctattcaggtttttgaagaaattggcaagcttattgggccaaagggggctgacaggtcacgtttgtttattatggatagcacctacatggctgataagaacccattgattgagcgcggtgcgccatatattgatttgttgcttgtgcaggtttatggtgctcaaggtgaacgaggagagtgggacccggttgctagaaagcctgaaaaaacaatggaggaacgatgggagtcgtatagcaaatacattcgtcctgagcagtacatggttggcttctccttctacgaggaaaatgcgggcagtggtaacctctggtatgacattaatgaaagaaaagatgatcataatccgttacattcagagataactggtactcgtgctgagcgctatgcaaaatggcagcctaagacaggtggtgttaagggagggattttctcttatgcaattgatcgtgacggcgtagcacatcaaccagaaaagtatgccaagcgtaaagattttaaagatgtaacagataagatattccactctgattacaaagtatcaaaggccctaaaagaggtcatggtaaaggacaaatcctatgagcagattgatgaaacagattttccagacaaggctttgcgagaagcagttattgcacaggttggaagcagaagaggagatttggagcgcttcaatggcaccctgcgcttagataacccagatatcaagagcttagaaggcctaaataagcttaaaaaactagctaagctagagctagttggtttatcgcaaatcacaaagctagatcaatcagtcctgcctgaaaatattaagccaaccaaggatacgctagtcagcgttcttgaagcctataaaaaagatgatcaggaagcagctaaggcgattccacaggtggctctgaccatttctggtctaactggcttgaaggaattaaatcttgctggctttgagcgtgagaccttggctggaattgacgcagctagcctaacctctcttgaaaaggtggatctctctagcaataagctggacttagcagctggcactgacaatcgtcagattcttgataccatgctggcaacagtgactaagcatggcaaagccaatgcagacaatatgacatttgatcatcaaaagcctaccggtctttatcctgatacttatggcactaagagccttcagttaccagtagcaaatgatacaattgatttgcaggctaagcttttatttggaacagttactaatcagggcacgctaatcaatagcgaagctgactataaggcttatcaggagcaggaaatagcaggtcgccgctttgttgatccaagctatgactacaaagcttttgcagtgacctacgatgcttataaggtcagagtgactgactcaaccttaggcgttactgatgagaagaagctctccactagcaaggaggaaacctacaagattgaattctttagccctactaatagcactaagcctgtgcatgaggctaaggttgtcgttggtgaagaaaaaaccatgatggtcaacctagcagagggagcaacgattattgggggaagtgcagatcaaacaaatgcaaaaaaagtgtttgatggtttgctcaataatgatacaacaactctgtcaactagcaataaagcttctatcatttttgaacttaaagagtctggcttagtcaagcattggcgtttctttaatgatagtgccaaaaagaaagaagattatatcaaggaagctaagcttgaagccttcgttggtcatcttgaggacagctcaaaggtgaaggatagcttagaaaaatcaactgaatgggtaacagtttcagattattcaggagaggctcaagagtttagccagccgttaaacaacgttggtgccaaatattggagaataacaattgacaataagaaaagtcaatatggatatgtctctcttcctgagctgcaacttattggttatcaattaccggctgcgtatcctgtgatggcaacgctagctgctgcagaggagctatctcaacaaaaagacaagttctctcaaaagcagcttaaggagctcgaagtcaaagtagctgccttaaaggctgctttagataataagatgtttaatgctgatactattaatgctagctttgctgatgtgaaagcttatgttgataagctactagcagacgcagctggcaagaaaacaccaggcaaagccactaaggaagctcagctagtgactactgacgcaaaagaaaaggctgagtcagaaaaatcaaaggcagac

SEQ ID NO: 13 - protein, 977 aa, Streptococcus equi
EDKVVQTSPSVSAIDDLHYLSENSKKEFKEELSKAGEVPEKLKEILSKAQQADKQAKTLAEMKVPEKIPMKPLKGPLYGGYFRTWHDKTSDPAEKDKVNSMGELPKEVDLAFVFHDWTKDYSLFWQKLATKHIPKLNKQGTRVIRTIPWRFLAGGDHSGIAEDAQKYPNTPEGNKALAKAIVDEYVYKYNLDGLDVDIERDSIPKVNKEESKEGIERSIQVFEEIGKLIGPKGADRSRLFIMDSTYMADKNPLIERGAPYIDLLLVQVYGAQGERGEWDPVARKPEKTMEERWESYSKYIRPEQYMVGFSFYEENAGSGNLWYDINERKDDHNPLHSEITGTRAERYAKWQPKTGGVKGGIFSYAIDRDGVAHQPEKYAKRKDFKDVTDKIFHSDYKVSKALKEVMVKDKSYEQIDETDFPDKALREAVIAQVGSRRGDLERFNGTLRLDNPDIKSLEGLNKLKKLAKLELVGLSQITKLDQSVLPENIKPTKDTLVSVLEAYKKDDQEAAKAIPQVALTISGLTGLKELNLAGFERETLAGIDAASLTSLEKVDLSSNKLDLAAGTDNRQILDTMLATVTKHGKANADNMTFDHQKPTGLYPDTYGTKSLQLPVANDTIDLQAKLLFGTVTNQGTLINSEADYKAYQEQEIAGRRFVDPSYDYKAFAVTYDAYKVRVTDSTLGVTDEKKLSTSKEETYKIEFFSPTNSTKPVHEAKVVVGEEKTMMVNLAEGATIIGGSADQTNAKKVFDGLLNNDTTTLSTSNKASIIFELKESGLVKHWRFFNDSAKKKEDYIKEAKLEAFVGHLEDSSKVKDSLEKSTEWVTVSDYSGEAQEFSQPLNNVGAKYWRITIDNKKSQYGYVSLPELQLIGYQLPAAYPVMATLAAAEELSQQKDKFSQKQLKELEVKVAALKAALDNKMFNADTINASFADVKAYVDKLLADAAGKKTPGKATKEAQLVTTDAKEKAESEKSKAD

SEQ ID NO: 14 - DNA, 2985 bp, Streptococcus pyogenes
atggataaacatttgttggtaaaaagaacactagggtgtgtttgtgctgcaacgttgatgggagctgccttagcgacccaccatgattcactcaatactgtaaaagcggaggagaagactgttcaggttcagaaaggattaccttctatcgatagcttgcattatctgtcagagaatagcaaaaaagaatttaaagaagaactctcaaaagcggggcaagaatctcaaaaggtcaaagagatattagcaaaagctcagcaggcagataaacaagctcaagaacttgccaaaatgaaaattcctgagaaaataccgatgaaaccgttacatggtcctctctacggtggttactttagaacttggcatgacaaaacatcagatccaacagaaaaagacaaagttaactcgatgggagagcttcctaaagaagtagatctagcctttattttccacgattggacaaaagattatagccttttttggaaagaattggccaccaaacatgtgccaaagttaaacaagcaagggacacgtgtcattcgtaccattccatggcgtttcctagctgggggtgataacagtggtattgcagaagataccagtaaatacccaaatacaccagagggaaataaagctttagccaaagctattgttgatgaatatgtttataaatacaaccttgatggcttagatgtggatgttgaacatgatagtattccaaaagttgacaaaaaagaagatacagcaggcgtagaacgctctattcaagtgtttgaagaaattgggaaattaattggaccaaaaggtgttgataaatcgcggttatttattatggatagcacctacatggctgataaaaacccattgattgagcgaggagctccttatattaatttattactggtacaggtctatggttcacaaggagagaaaggtggttgggagcctgtttctaatcgacctgaaaaaacaatggaagaacgatggcaaggttatagcaagtatattcgtcctgaacaatacatgattggtttttctttctatgaggaaaatgctcaagaagggaatctttggtatgatattaattctcgcaaggacgaggacaaagcaaatggaattaacactgacataactggaacgcgtgccgaacggtatgcaaggtggcaacctaagacaggtggggttaagggaggtatcttctcctacgctattgaccgagatggtgtagctcatcaacctaaaaaatatgctaaacagaaagagtttaaggacgcaactgataacatcttccactcagattatagtgtctccaaggcattaaagacagttatgctaaaagataagtcgtatgatctgattgatgagaaagatttcccagataaggctttgcgagaagctgtgatggcgcaggttggaaccagaaaaggtgatttggaacgtttcaatggcacattacgattggataatccagcgattcaaagtttagaaggtctaaataaatttaaaaaattagctcaattagacttgattggcttatctcgcattacaaagctcgaccgttctgttttacccgctaatatgaagccaggcaaagataccttggaaacagttcttgaaacctataaaaaggataacaaagaagaacctgctactatcccaccagtatctttgaaggtttctggtttaactggtctgaaagaattagatttgtcaggttttgaccgtgaaaccttggctggtcttgatgccgctactctaacgtctttagaaaaagttgatatttctggcaacaaacttgatttggctccaggaacagaaaatcgacaaatttttgatactatgctatcaactatcagcaatcatgttggaagcaatgaacaaacagtgaaatttgacaagcaaaaaccaactgggcattacccagatacctatgggaaaactagtctgcgcttaccagtggcaaatgaaaaagttgatttgcaaagccagcttttgtttgggactgtgacaaatcaaggaaccctaatcaatagcgaagcagactataaggcttaccaaaatcataaaattgctggacgtagctttgttgattcaaactatcattacaataactttaaagtttcttatgagaactataccgttaaagtaactgattccacattgggaaccactactgacaaaacgctagcaactgataaagaagagacctataaggttgacttctttagcccagcagataagacaaaagctgttcatactgctaaagtgattgttggtgacgaaaaaaccatgatggttaatttggcagaaggcgcaacagttattggaggaagtgctgatcctgtaaatgcaagaaaggtatttgatgggcaactgggcagtgagactgataatatctctttaggatgggattctaagcaaagtattatatttaaattgaaagaagatggattaataaagcattggcgtttcttcaatgattcagcccgaaatcctgagacaaccaataaacctattcaggaagcaagtctacaaatttttaatatcaaagattataatctagataatttgttggaaaatcccaataaatttgatgatgaaaaatattggattactgtagatacttacagtgcacaaggagagagagctactgcattcagtaatacattaaataatattactagtaaatattggcgagttgtctttgatactaaaggagatagatatagttcgccagtagtccctgaactccaaattttaggttatccgttacctaacgccgacactatcatgaaaacagtaactactgctaaagagttatctcaacaaaaagataagttttctcaaaagatgcttgatgagttaaaaataaaagagatggctttagaaacttctttgaacagtaagatttttgatgtaactgctattaatgctaatgctggagttttgaaagattgtattgagaaaaggcagctgctaaaaaaa

SEQ ID NO: 15 - protein, 995 aa, Streptococcus pyogenes
MDKHLLVKRTLGCVCAATLMGAALATHHDSLNTVKAEEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 16 - DNA, 2877 bp, Streptococcus pyogenes
gaggagaagactgttcaggttcagaaaggattaccttctatcgatagcttgcattatctgtcagagaatagcaaaaaagaatttaaagaagaactctcaaaagcggggcaagaatctcaaaaggtcaaagagatattagcaaaagctcagcaggcagataaacaagctcaagaacttgccaaaatgaaaattcctgagaaaataccgatgaaaccgttacatggtcctctctacggtggttactttagaacttggcatgacaaaacatcagatccaacagaaaaagacaaagttaactcgatgggagagcttcctaaagaagtagatctagcctttattttccacgattggacaaaagattatagccttttttggaaagaattggccaccaaacatgtgccaaagttaaacaagcaagggacacgtgtcattcgtaccattccatggcgtttcctagctgggggtgataacagtggtattgcagaagataccagtaaatacccaaatacaccagagggaaataaagctttagccaaagctattgttgatgaatatgtttataaatacaaccttgatggcttagatgtggatgttgaacatgatagtattccaaaagttgacaaaaaagaagatacagcaggcgtagaacgctctattcaagtgtttgaagaaattgggaaattaattggaccaaaaggtgttgataaatcgcggttatttattatggatagcacctacatggctgataaaaacccattgattgagcgaggagctccttatattaatttattactggtacaggtctatggttcacaaggagagaaaggtggttgggagcctgtttctaatcgacctgaaaaaacaatggaagaacgatggcaaggttatagcaagtatattcgtcctgaacaatacatgattggtttttctttctatgaggaaaatgctcaagaagggaatctttggtatgatattaattctcgcaaggacgaggacaaagcaaatggaattaacactgacataactggaacgcgtgccgaacggtatgcaaggtggcaacctaagacaggtggggttaagggaggtatcttctcctacgctattgaccgagatggtgtagctcatcaacctaaaaaatatgctaaacagaaagagtttaaggacgcaactgataacatcttccactcagattatagtgtctccaaggcattaaagacagttatgctaaaagataagtcgtatgatctgattgatgagaaagatttcccagataaggctttgcgagaagctgtgatggcgcaggttggaaccagaaaaggtgatttggaacgtttcaatggcacattacgattggataatccagcgattcaaagtttagaaggtctaaataaatttaaaaaattagctcaattagacttgattggcttatctcgcattacaaagctcgaccgttctgttttacccgctaatatgaagccaggcaaagataccttggaaacagttcttgaaacctataaaaaggataacaaagaagaacctgctactatcccaccagtatctttgaaggtttctggtttaactggtctgaaagaattagatttgtcaggttttgaccgtgaaaccttggctggtcttgatgccgctactctaacgtctttagaaaaagttgatatttctggcaacaaacttgatttggctccaggaacagaaaatcgacaaatttttgatactatgctatcaactatcagcaatcatgttggaagcaatgaacaaacagtgaaatttgacaagcaaaaaccaactgggcattacccagatacctatgggaaaactagtctgcgcttaccagtggcaaatgaaaaagttgatttgcaaagccagcttttgtttgggactgtgacaaatcaaggaaccctaatcaatagcgaagcagactataaggcttaccaaaatcataaaattgctggacgtagctttgttgattcaaactatcattacaataactttaaagtttcttatgagaactataccgttaaagtaactgattccacattgggaaccactactgacaaaacgctagcaactgataaagaagagacctataaggttgacttctttagcccagcagataagacaaaagctgttcatactgctaaagtgattgttggtgacgaaaaaaccatgatggttaatttggcagaaggcgcaacagttattggaggaagtgctgatcctgtaaatgcaagaaaggtatttgatgggcaactgggcagtgagactgataatatctctttaggatgggattctaagcaaagtattatatttaaattgaaagaagatggattaataaagcattggcgtttcttcaatgattcagcccgaaatcctgagacaaccaataaacctattcaggaagcaagtctacaaatttttaatatcaaagattataatctagataatttgttggaaaatcccaataaatttgatgatgaaaaatattggattactgtagatacttacagtgcacaaggagagagagctactgcattcagtaatacattaaataatattactagtaaatattggcgagttgtctttgatactaaaggagatagatatagttcgccagtagtccctgaactccaaattttaggttatccgttacctaacgccgacactatcatgaaaacagtaactactgctaaagagttatctcaacaaaaagataagttttctcaaaagatgcttgatgagttaaaaataaaagagatggctttagaaacttctttgaacagtaagatttttgatgtaactgctattaatgctaatgctggagttttgaaagattgtattgagaaaaggcagctgctaaaaaaa

SEQ ID NO: 17 - protein, 959 aa, Streptococcus pyogenes
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 18 - DNA, 32 bp, Artificial Sequence
  Primer
gtcggatccgaggataaggttgtgcaaactag

SEQ ID NO: 19 - DNA, 32 bp, Artificial Sequence
  Primer
gcctctcgagggataagctagtctgtctttgg

SEQ ID NO: 20 - DNA, 35 bp, Artificial Sequence
  Primer
gcagctcgagttaatattgggcaccgcgctcaatc

SEQ ID NO: 21 - DNA, 30 bp, Artificial Sequence
  Primer
tgacggatccaaggaggccaagcttgaagc

SEQ ID NO: 22 - DNA, 28 bp, Artificial Sequence
  Primer
ttatggatccgatcgcgatggtgtagcg

SEQ ID NO: 23 - DNA, 33 bp, Artificial Sequence
  Primer
tcttctcgagttaaccgccatgcttagtcactg

SEQ ID NO: 24 - DNA, 23 bp, Artificial Sequence
  Primer
gggctggcaagccacgtttggtg

SEQ ID NO: 25 - DNA, 23 bp, Artificial Sequence
  Primer
ccgggagctgcatgtgtcagagg

SEQ ID NO: 26 - protein, 1011 aa, Streptococcus equi
MEKQVLVKKTLKCVCAAALMVAILAAQHDSLVRVKAEDKLVQTSPSVSAIDDLHYLSENSKKEFKEELSKVEKAQPEKLKEIVSKAQRANQQAKTLAEMKIPEKIPMKPLKGPLYGGYFRTWHDKTSDPAEKDKVNSMGELPKEVDLAFVFHDWTKDYSLFWQELATKHVPTLNKQGTRVIRTIPWRFLAGGDHSGIAEDAQKYPNTPEGNKALAKAIVDEYVYKYNLDGLDVDVERDSIPKVNGQESNANIQRSIAVFEEIGKLIGPKGADRSRLFIMDSTYMADKNPLIERGAPYIDLLLVQVYGAQGEKGGFDNANHKAVDTMEERWESYSKYIRPEQYMVGFSFYEEKANSGNLWYDVNVEDDTNPNIGSEIKGTRAERYAKWQPKTGGVKGGIFSYGIDRDGVAHPKKNGPKTPDLDKIVKSDYKVSKALKKVMENDKSYELIDETDFPDKALREAVIAQVGSRRGDLERFNGTLRLDNPAIQSLEGLNKLKKLAKLELIGLSQITKLDSLVLPANAKPTKDTLVSGLETYKNDDRKEEAKAIPQVALTISGLTGLKELNLAGFERETLAGIDAASLTSLEKVDLSKNKLDLAAGTENRQILDTMLATVTKHGGVSEKTFVFDHQKPTGLYPDTYGTKSLQLPVANDTIDLQAKLLFGTVTNQGTLINSEADYKAYQEQEIAGHRFVDSSYDYKAFAVTYKDYKIKVTDSTLGVTDHKDLSTSKEETYKVEFFSPTNSTKPVHEAKVVVGEEKTMMVNLAEGATIIGGSADQTNAKKVFDGLLNNDTTTLSTSNKASIIFELKESGLVKHWRFFNDSAKKKEDYIKEAKLEAFVGHLEDSSKVKDSLEKSTEWVTVSDYSGEAQEFSQPLNNVGAKYWRITIDNKKSQYGYVSLPELQLIGYQLPAAYPVMATLAAAEELSQQKDKFSQKQLKELEVKVAALKAALDNKMFNADTINASFADVKAYVDKLLADAAGKKTPGKATKEAQLVTTDAKEKAESEKSKAN